(19) United States
(12) Patent Application Publication
(10) Pub. No.: US 2021/0000838 A1
(43) Pub. Date: Jan. 7, 2021

(54) COMBINATION THERAPY

(71) Applicant: MEI Pharma, Inc., San Diego, CA (US)

(72) Inventor: Daniel P. GOLD, San Diego, CA (US)

(21) Appl. No.: 16/981,780

(22) PCT Filed: Mar. 20, 2019
(86) PCT No.: PCT/US19/23172

§ 371 Date: Sep. 17, 2020

Related U.S. Application Data
(60) Provisional application No. 62/646,314, filed on Mar. 21, 2018.

(51) Int. Cl. A61K 31/5377; A61K 31/519

(52) U.S. Cl. CPC A61K 31/5377; A61K 9/20; A61K 31/519

(57) ABSTRACT

Provided herein are methods of treating diseases, such as cancer, using a combination therapy. In certain embodiments, the methods comprise administering an effective amount of a phosphoinositide-3-kinase (PI3K) inhibitor and an effective amount of a Bruton tyrosine kinase (BTK) inhibitor to a patient.

### CROSS REFERENCE TO RELATED APPLICATIONS

This application claims the benefit of U.S. Provisional Application No. 62/646,314, filed Mar. 21, 2018, which is incorporated by reference in the disclosure of this application.

### FIELD

Provided herein are methods of treating diseases using a combination therapy for treatment of a proliferative disease, including a cancer, an autoimmune disease and an inflammatory disease. In certain embodiments, the methods comprise administering an effective amount of a phosphoinositide-3-kinase (PI3K) inhibitor and an effective amount of Bruton tyrosine kinase (BTK) inhibitor to a patient.

### BACKGROUND OF THE INVENTION

Phosphoinositide-3-kinases (PI3Ks) play a variety of roles in normal tissue physiology with p110α having a specific role in cancer growth, p11013 in thrombus formation mediated by integrin α_{π}β₃, and p110γ in inflammation, rheumatoid arthritis, and other chronic inflammation states. Inhibitors of PI3K have therapeutic potential in the treatment of various proliferative diseases, including cancer.

The Bruton's tyrosine kinase (BTK) inhibitors are a class of drugs that inhibit Bruton tyrosine kinase (BTK), a member of the Tec family of kinases with a very distinct role in B-cell antigen receptor (SCR) signaling.

There continues to be a need for development of effective dosages and dosing regimens for administering PI3K inhibitors and BTK inhibitors in combination with a second agent in treating, preventing and managing various proliferative diseases, including cancers, autoimmune diseases and/or inflammatory diseases.

### SUMMARY OF THE INVENTION

Disclosed herein is a method for treating or preventing cancer, comprising administering to a patient in need thereof:
(i) about 30 mg, about 45 mg, or about 60 ma of a compound of Formula (I):

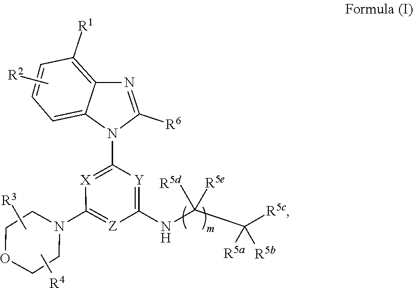

or an enantiomer, a mixture of enantiomers, a mixture of two or more diastereomers, or an isotopic variant thereof; or a pharmaceutically acceptable salt, solvate, hydrate, or prodrug thereof; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, four, or five substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{h}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{h}R^{c}, —OS(O)₂NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{h}R^{c}, —SR^{a}, —S(O)R^{a}, —S(O)₂R^{a}, —S(O)NR^{h}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four, substituents Q^{a}; or (iii) R^{b} and R^{c} together with the N atom to which they are attached form heterocyclyl, which is further optionally substituted with one, two, three, or four, substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein two substituents Q that are adjacent to each other optionally form a C₃₋₁₀ cycloalkenyl, C₆₋₁₄ aryl, heteroaryl, or heterocyclyl, each optionally substituted with one, two, three, or four substituents Q^{a}; and
(ii) about 160 mg or about 320 mg of BGB-3111, or a pharmaceutically acceptable salt thereof, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day.

In some embodiments, R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments, R^{5a} and R^{5b} are each independently (a) halo; (b) C₁₋₆ alkyl, C₂₋₆ alkenyl, C₂-6 alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

In some embodiments, R^{5a} and R^{5b} are each methyl, optionally substituted with one, two, or three halo(s). In some embodiments, n is 1. In some embodiments, n is 1 and R^{5f} and R^{5g} are each hydrogen. In some embodiments, n is 0. In some embodiments, m is 0.

Synthesis of compounds of Formula (I) is described in U.S. Pat. No. 9,056,852 B2, which is incorporated by reference for such disclosure.

In some embodiments, the compound of Formula (I) is of Formula (XI):

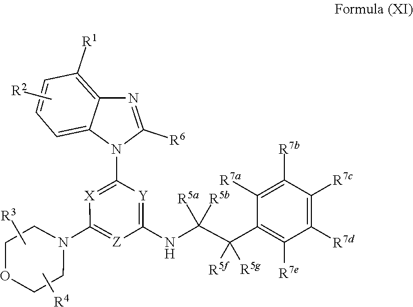

or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —S(O)R^{a}, —S(O)₂R^{a}, —S(O)NR^{b}R^{c}, —S(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

Synthesis of compounds of Formula (XI) is described in U.S. Pat. No. 9,056,852 B2, which is incorporated by reference for such disclosure

In some embodiments, the compound of Formula (I) is Compound I:

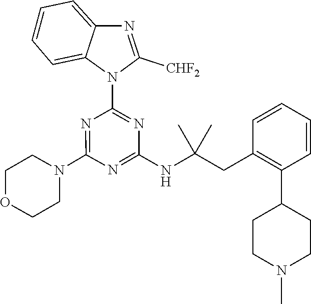 [SMILES: CN1CCC(c2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof.

In some embodiments, the compound of Formula (I) is Compound II:

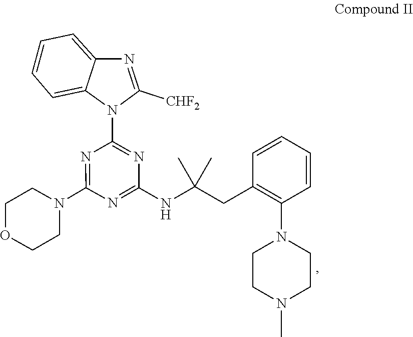 [SMILES: CN1CCN(c2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof.

In some embodiments, the compound of Formula (I) is Compound III:

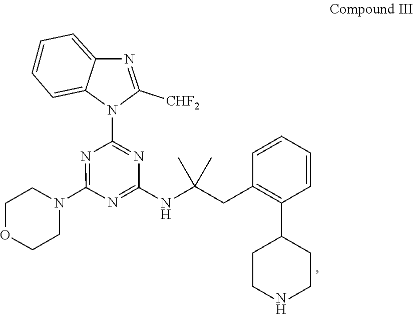 [SMILES: CC(C)(Cc1ccccc1C1CCNCC1)Nc1nc(N2CCOCC2)nc(-n2c(C(F)F)nc3ccccc32)n1]

an isotopic variant thereof, a pharmaceutically acceptable salt, solvate, hydrate, or prodrug thereof.

In some embodiments, the compound of Formula (I) is Compound IV:

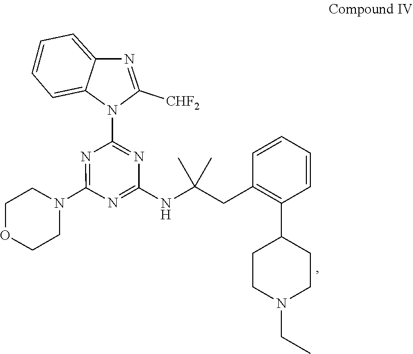 [SMILES: CCN1CCC(c2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof.

In some embodiments, the compound of Formula (I) is Compound V:

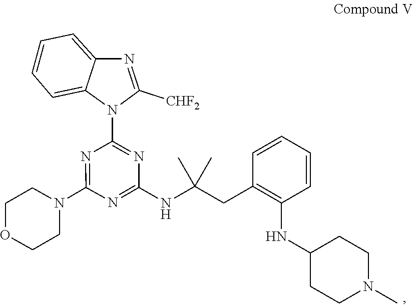 [SMILES: CN1CCC(Nc2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof.

In some embodiments, the compound of Formula (I) is Compound VI:

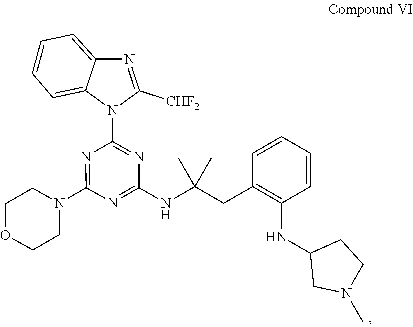 [SMILES: CN1CCC(Nc2ccccc2CC(C)(C)Nc2nc(N3CCOCC3)nc(-n3c(C(F)F)nc4ccccc43)n2)C1]

an isotopic variant thereof, a pharmaceutically acceptable salt, solvate, hydrate, or prodrug thereof.

In some embodiments, the compound of Formula (I) is Compound VII:

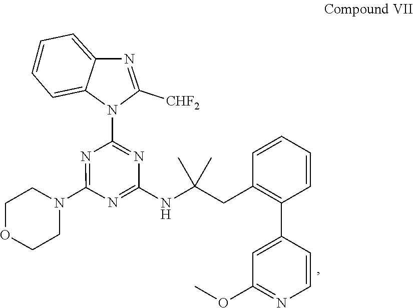 [SMILES: COc1cc(-c2ccccc2CC(C)(C)Nc2nc(N3CCOCC3)nc(-n3c(C(F)F)nc4ccccc43)n2)ccn1]

an isotopic variant thereof, a pharmaceutically acceptable salt, solvate, hydrate, or prodrug thereof.

In some embodiments, the compound of Formula (I) is Compound VIII:

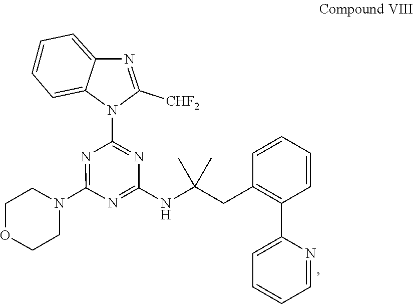 [SMILES: CC(C)(Cc1ccccc1-c1ccccn1)Nc1nc(N2CCOCC2)nc(-n2c(C(F)F)nc3ccccc32)n1]

an isotopic variant thereof, a pharmaceutically acceptable salt, solvate, hydrate, or prodrug thereof.

In some embodiments, the compound of Formula (I) is Compound IX:

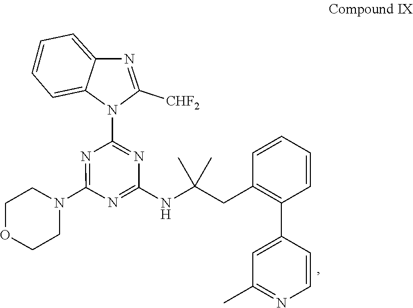 [SMILES: Cc1cc(-c2ccccc2CC(C)(C)Nc2nc(N3CCOCC3)nc(-n3c(C(F)F)nc4ccccc43)n2)ccn1]

an isotopic variant thereof, a pharmaceutically acceptable salt, solvate, hydrate, or prodrug thereof.

In some embodiments, the compound of Formula (I) is Compound X:

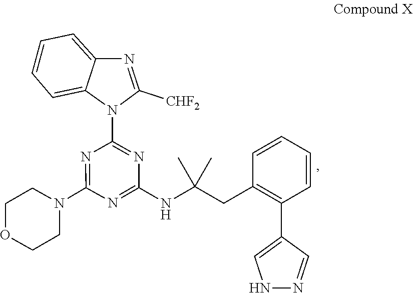 [SMILES: CC(C)(Cc1ccccc1-c1cn[nH]c1)Nc1nc(N2CCOCC2)nc(-n2c(C(F)F)nc3ccccc32)n1]

an isotopic variant thereof, a pharmaceutically acceptable salt, solvate, hydrate, or prodrug thereof.

In some embodiments, the cancer being treated is a hematological malignancy. In some embodiments, the cancer being treated is a B-cell malignancy. In some embodiments, the cancer being treated is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the cancer being treated is chronic lymphocytic leukemia or non-Hodgkin's lymphoma. In some embodiments, the cancer being treated is non-Hodgkin's lymphoma and the non-Hodgkin's lymphoma is diffuse large B-cell lymphoma (DLBCL). In some embodiments, the cancer being treated is relapsed-refractory diffuse large B-cell lymphoma (r/r DLBCL). In some embodiments, the diffuse large B-cell lymphoma is of the activated B-cell (ABC DLBCL) or Germinal center B-cell (GCB DLBCL).

In some embodiments, about 30 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject. In some embodiments, about 45 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject. In some embodiments, about 60 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

In some embodiments, about 160 mg of BGB-3111, or a pharmaceutically acceptable salt thereof, is administered to the subject. In some embodiments, about 320 mg of BGB-3111, or a pharmaceutically acceptable salt thereof, is administered to the subject. In some embodiments, BGB-3111, or a pharmaceutically acceptable salt thereof, is administered to the subject once per day or twice per day. In some embodiments, BGB-3111, or a pharmaceutically acceptable salt thereof, is administered to the subject once per day. In some embodiments, BGB-3111, or a pharmaceutically acceptable salt thereof, is administered to the subject twice per day. In some embodiments, about 160 mg of BGB-3111, or a pharmaceutically acceptable salt thereof, is administered to the subject twice per day. In some embodiments, about 320 mg of BGB-3111, or a pharmaceutically acceptable salt thereof, is administered to the subject once per day.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, and BGB-3111, or a pharmaceutically acceptable salt thereof, are administered simultaneously, approximately simultaneously, or sequentially in any order.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, and BGB-3111, or a pharmaceutically acceptable salt thereof, are administered simultaneously or approximately simultaneously.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, and BGB-3111, or a pharmaceutically acceptable salt thereof, are administered sequentially.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered before BGB-3111, or a pharmaceutically acceptable salt thereof.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered after BGB-3111, or a pharmaceutically acceptable salt thereof.

In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule. In some embodiments, BGB-3111, or a pharmaceutically acceptable salt thereof, is formulated as a tablet or capsule. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is co-formulated with BGB-3111, or a pharmaceutically acceptable salt thereof.

Disclosed herein is a pharmaceutical composition, comprising Compound I:

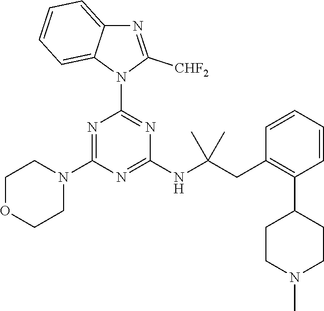 [SMILES: CN1CCC(c2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof; BGB-3111, or a pharmaceutically acceptable salt thereof, and a pharmaceutically acceptable excipient.

Disclosed herein is a pharmaceutical composition, comprising Compound II:

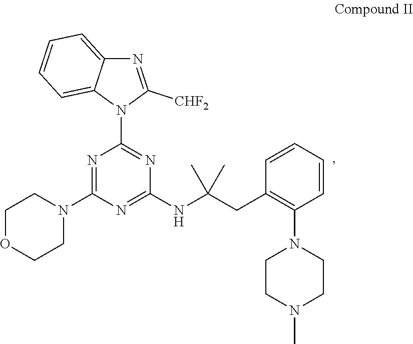 [SMILES: CN1CCN(c2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof; BGB-3111, or a pharmaceutically acceptable salt thereof, and a pharmaceutically acceptable excipient.

Disclosed herein is a pharmaceutical composition, comprising Compound III:

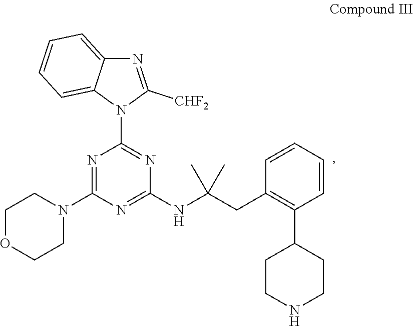 [SMILES: CC(C)(Cc1ccccc1C1CCNCC1)Nc1nc(N2CCOCC2)nc(-n2c(C(F)F)nc3ccccc32)n1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound IV:

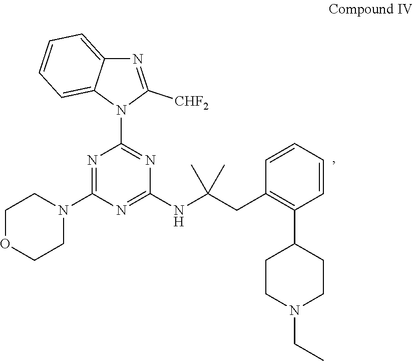 [SMILES: CCN1CCC(c2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound V:

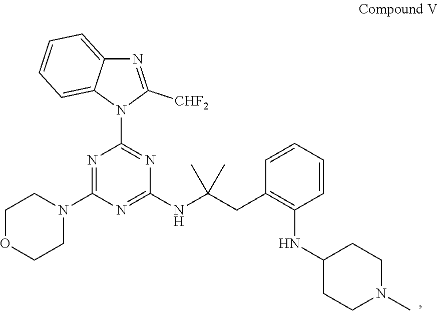 [SMILES: CN1CCC(Nc2ccccc2CC(C)(C)Nc2nc(N3CCOCC3)nc(-n3c(C(F)F)nc4ccccc43)n2)CC1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound VI:

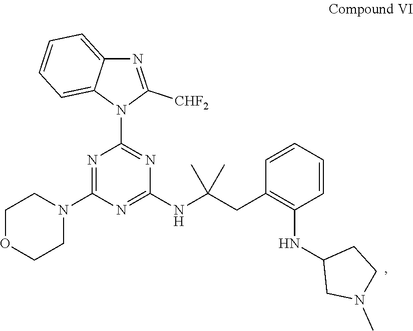 [SMILES: CN1CCC(Nc2ccccc2CC(C)(C)Nc2nc(N3CCOCC3)nc(-n3c(C(F)F)nc4ccccc43)n2)C1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound VII:

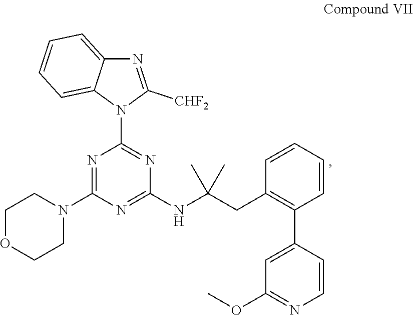 [SMILES: COc1cc(-c2ccccc2CC(C)(C)Nc2nc(N3CCOCC3)nc(-n3c(C(F)F)nc4ccccc43)n2)ccn1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound VIII:

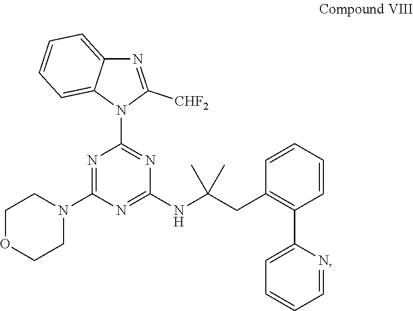 [SMILES: CC(C)(Cc1ccccc1-c1ccccn1)Nc1nc(N2CCOCC2)nc(-n2c(C(F)F)nc3ccccc32)n1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound IX:

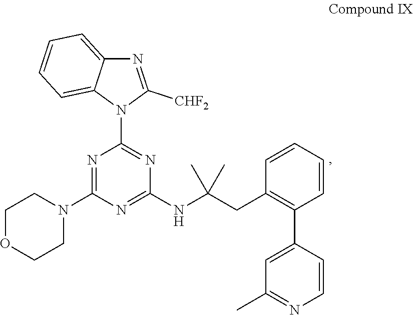 [SMILES: Cc1cc(-c2ccccc2CC(C)(C)Nc2nc(N3CCOCC3)nc(-n3c(C(F)F)nc4ccccc43)n2)ccn1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a pharmaceutical composition, comprising Compound X:

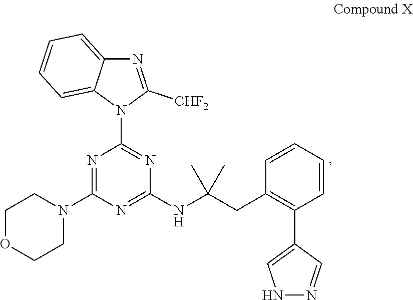 [SMILES: CC(C)(Cc1ccccc1-c1cn[nH]c1)Nc1nc(N2CCOCC2)nc(-n2c(C(F)F)nc3ccccc32)n1]

an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound I, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound II, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound III, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound IV, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound V, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound VI, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound VII, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound VIII, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound IX, an isotopic variant thereof, a pharmaceutically acceptable salt, solvate, hydrate, or prodrug thereof; BGB-3111, or a pharmaceutically acceptable salt thereof; and a pharmaceutically acceptable excipient.

Disclosed herein is a method of treating or preventing cancer, comprising administering to a subject in need thereof an effective amount of a pharmaceutical composition, comprising Compound X, an isotopic variant thereof, a pharmaceutically acceptable salt, solvate, hydrate, or prodrug thereof; BGB-3111, or a pharmaceutically acceptable salt thereof; and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates % growth inhibition vs. concentration of compound I in DB cells as measured in ATPLite assay.

FIG. 2 illustrates % growth inhibition vs. concentration of compound I in DOHH-2 cells as measured in ATPLite assay.

FIG. 3 illustrates % growth inhibition vs. concentration of compound I in HT cells as measured in ATPLite assay.

FIG. 4 illustrates % growth inhibition vs. concentration of compound I in NU-DHL-1 cells as measured in ATPLite assay.

FIG. 5 illustrates % growth inhibition vs. concentration of compound I in OCI-Ly19 cells as measured in ATPLite assay.

FIG. 6 illustrates % growth inhibition vs. concentration of compound I in OCI-Ly3 cells as measured in ATPLite assay.

FIG. 7 illustrates % growth inhibition vs. concentration of compound I in Pfeiffer cells as measured in ATPLite assay.

FIG. 8 illustrates % growth inhibition vs. concentration of compound I in SU-DHL-10 cells as measured in ATPLite assay.

FIG. 9 illustrates growth inhibition (GI₅₀) for compound I in the tested cell lines.

FIG. 10 illustrates % growth inhibition for compound I in the tested cell lines.

### DETAILED DESCRIPTION OF THE INVENTION

Described herein are pharmaceutical compositions comprising i) a PI3K inhibitor; and ii) a BTK inhibitor. In some instances, the pharmaceutical compositions described herein may be used for treating diseases or disorders such as cancer. Also described herein are methods of treating the diseases and disorders such as cancer with a combination of i) a PI3K inhibitor, and; ii) a BTK inhibitor.

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

### Definitions

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “therapeutically effective amount” and “effective amount” are meant to include the amount of a compound that, when administered, is sufficient to prevent development of, or alleviate to some extent, one or more of the symptoms of the disorder, disease, or condition being treated. The terms “therapeutically effective amount” or “effective amount” also refer to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The terms “pharmaceutically acceptable carrier,” “pharmaceutically acceptable excipient,” “physiologically acceptable carrier,” and “physiologically acceptable excipient” refer to a pharmaceutically-acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, Pa., 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, Fla., 2009.

The terms “about” and “approximately” mean an acceptable error for a particular value as determined by one of ordinary skill in the art, which depends in part on how the value is measured or determined. In certain embodiments, the terms “about” and “approximately” mean within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition.

The terms “naturally occurring” and “native” when used in connection with biological materials such as nucleic acid molecules, polypeptides, host cells, and the like, refer to materials which are found in nature and are not manipulated by man. Similarly, “non-naturally occurring” or “non-native” refers to a material that is not found in nature or that has been structurally modified or synthesized by man.

The term “PI3K” refers to a phosphoinositide 3-kinase or variant thereof, which is capable of phosphorylating the inositol ring of PI in the D-3 position. The term “PI3K variant” is intended to include proteins substantially homologous to a native PI3K, i.e., proteins having one or more naturally or non-naturally occurring amino acid deletions, insertions, or substitutions (e.g., PI3K derivatives, homologs, and fragments), as compared to the amino acid sequence of a native PI3K. The amino acid sequence of a PI3K variant is at least about 80% identical, at least about 90% identical, or at least about 95% identical to a native PI3K. Examples of PI3K include, but are not limited to, p110α, p110β, p110δ, p110γ, PI3K-C2α, PI3K-C2β, PI3K-C2γ, Vps34, mTOR, ATM, ATR, and DNA-PK. See, Fry, Biochem. Biophys. Acta 1994, 1226, 237-268; Vanhaesebroeck and Waterfield, Exp. Cell. Res. 1999, 253, 239-254; and Fry, Breast Cancer Res. 2001, 3, 304-312. PI3Ks are classified into at least four classes. Class I includes p110α, p110β, p110δ, and p110γ. Class II includes PI3K-C2α, PI3K-C2β, and PI3K-C2γ. Class III includes Vps34. Class IV includes mTOR, ATM, ATR, and DNA-PK. In certain embodiments, the PI3K is a Class I kinase. In certain embodiments, the PI3K is p110α, p110β, p110δ, or p110γ. In certain embodiments, the PI3K is a variant of a Class I kinase. In certain embodiments, the PI3K is a p110α mutant. Examples of p110α mutants include, but are not limited to, R38H, G106V, K111N, K227E, N345K, C420R, P539R, E542K, E545A, E545G, E545K, Q546K, Q546P, E453Q, H710P, 1800L, T1025S, M10431, M1043V, H1047L, H1047R, and H1047Y (Ikenoue et al., Cancer Res. 2005, 65, 4562-4567; Gymnopoulos et al., Proc. Natl. Acad Sci., 2007, 104, 5569-5574). In certain embodiments, the PI3K is a Class II kinase. In certain embodiments, the PI3K is PI3K-C2α, PI3K-C2β, or PI3K-C2γ. In certain embodiments, the PI3K is a Class III kinase. In certain embodiments, the PI3K is Vps34. In certain embodiments, the PI3K is a Class IV kinase. In certain embodiments, the PI3K is mTOR, ATM, ATR, or DNA-PK.

As used herein, “BTK” refers to Bruton's tyrosine kinase. BTK belongs to the Tec tyrosine kinase family (Vetrie et al., Nature 361: 226-233, 1993; Bradshaw, Cell Signal. 22: 1175-84, 2010). BTK is primarily expressed in most hematopoietic cells such as B cells, mast cells and macrophages (Smith et al., J. Immunol. 152: 557-565, 1994) and is localized in bone marrow, spleen and lymph node tissue. BTK plays important roles in B-cell receptor (BCR) and FcR signaling pathways, which involve in B-cell development, differentiation (Khan, Immunol. Res. 23: 147, 2001. The BTK inhibitor may be selected from the compounds disclosed in U.S. Pat. Nos. 8,084,620B2; 7,514,444B2; 7,718,662B1; and 7,393,848B1; US Publication Nos. 2016/0083392A1; 2015/0005277A1; 2015/0259354A1; 2012/053189A1; 2010/254905A1; 2008/0139582A1; 2012/0077832A1; 2012/0232054A1; 2012/082702A1; 2010/0160303A1, 2012/129852A1; 2006/0178367A1; 2006/0183746A1; 2012/040961A1; 2010/144705A1; 2012/0028981A1; 2012/058996A1; 2009/0318448A1; 2010/0016301A1; 2009/105209A1; 2010/0222325A1; and 2010/0004231 A1; International Publication Nos. WO 2011/153514; WO 2011/046964; WO 2010/009342; WO 2008/121742; WO 2008/054827; WO 2007/087068; WO 2011/090760; WO 2010/028236; WO 2009/158571; WO 2009/051822, WO 2010/123870; WO 2010/126960; WO 2011/162515; WO 2012/135801; WO 2011/152351; WO 2007/136790A2; WO 2002/050071; WO 2008/116064; WO 2010/011837; WO 2011/159857, WO 2011/019780, WO 2011/029043; WO 2011/029046; WO 2005/005429; WO 2005/014599; WO 2005/047290; WO 2006/053121; WO 2008/033834; WO 2008/033858; WO 2006/099075; WO 2008/033854; WO 2008/033857; WO 2009/039397, WO 2009/137596; WO 2010/056875; WO 2010/068788; WO 2010/068806; WO 2010/068810, WO 2011/140488; WO 2012/030990; WO 2012/031004; WO 2005/011597; WO 2008/045627; WO 2008/144253, WO 2007/140222, WO 2013/008095, WO 2012/170976A2, WO 2012/135944A1; WO 2010/065898A2; WO 2012/158795A1; WO 2012/158764A1; WO 2012/158810A1; WO 2012156334A1; WO 2012020008; WO 2010122038; WO 2010006970; WO 2010006947; WO 2010000633; WO 2009077334; WO 2009098144, WO 2006065946; WO 2007027594; WO 2007027729 and EP2068849. The BTK inhibitor may also be selected from ibrutinib, BGB-3111, CC-292 (AVL-292), ACP 196 (Acalabrutinib), CNX-774, CGI1746, LFM-A13, CNX-774, ONO-4059, RN486 CPI-0610, DUAL946, GSK525762, I-BET151, JQ1, OTX015, PFI-1, RVX-208, RVX2135, TEN-010, and a combination thereof. In one embodiment, the BTK inhibitor is ibrutinib or BGB-3111.

The terms “synergy,” “synergism,” and “synergistic” as used herein refer to a combination of therapies (e.g., use of a PI3K inhibitor of Formula (I) and a BTK inhibitor) that is more effective than the expected additive effects of any two or more single therapies. For example, a synergistic effect of a combination of therapies permits the use of lower dosages of one or more of the therapies and/or less frequent administration of said therapies to a subject. The ability to utilize lower dosages of therapies and/or to administer the therapies less frequently reduces the toxicity associated with the administration of the therapies to a subject without reducing the efficacy of said therapies in the prevention, management, treatment, or amelioration of a given disease, such as an autoimmune disease, inflammatory disease, or cancer including, but not limited to, chronic lymphocytic leukemia or non-Hodgkin's lymphoma. In addition, a synergistic effect can result in improved efficacy of therapies in the prevention, management, treatment, or amelioration of a given disease, such an autoimmune disease, inflammatory disease, or cancer including, but not limited to, chronic lymphocytic leukemia or non-Hodgkin's lymphoma. Finally, synergistic effects of a combination of therapies may avoid or reduce adverse or unwanted side effects associated with the use of any single therapy. The “synergy,” “synergism,” or “synergistic” effect of a combination may be determined herein by the methods of Chou et al., and/or Clarke et al. See Ting-Chao Chou, Theoretical Basis, Experimental Design, and Computerized Simulation of Synergism and Antagonism in Drug Combination Studies, Pharmacol Rev 58:621-681 (2006), and Clarke et al., Issues in experimental design and endpoint analysis in the study of experimental cytotoxic agents in vivo in breast cancer and other models, Breast Cancer Research and Treatment 46:255-278 (1997), which are both incorporated by reference for the methods of determining the “synergy,” synergism,” or “synergistic” effect of a combination.

The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I) iodine-125 (¹²⁵I) iodine-127 (¹²⁷I) iodine-129 (¹²⁹I), and iodine-131 (¹³¹I) In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C) nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I) iodine-125 (¹²⁵I) iodine-129 (¹²⁹I), and iodine-131 (¹³¹I). It will be understood that, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a compound contains unnatural proportions of deuterium (D).

The term “alkyl” refers to a linear or branched saturated monovalent hydrocarbon radical, wherein the alkylene may optionally be substituted with one or more substituents Q as described herein. The term “alkyl” also encompasses both linear and branched alkyl, unless otherwise specified. In certain embodiments, the alkyl is a linear saturated monovalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms). For example, C₁₋₆ alkyl refers to a linear saturated monovalent hydrocarbon radical of 1 to 6 carbon atoms or a branched saturated monovalent hydrocarbon radical of 3 to 6 carbon atoms.

The term “alkylene” refers to a linear or branched saturated divalent hydrocarbon radical, wherein the alkylene may optionally be substituted with one or more substituents Q as described herein. The term “alkylene” encompasses both linear and branched alkylene, unless otherwise specified. In certain embodiments, the alkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms). For example, C₁₋₆ alkylene refers to a linear saturated divalent hydrocarbon radical of 1 to 6 carbon atoms or a branched saturated divalent hydrocarbon radical of 3 to 6 carbon atoms.

The term “heteroalkylene” refers to a linear or branched saturated divalent hydrocarbon radical that contains one or more heteroatoms each independently selected from O, S, and N in the hydrocarbon chain. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₁₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, CH₂O, CH₂OCH₂, CH₂CH₂O, CH₂NH, CH₂NHCH₂, CH₂CH₂NH, CH₂SCH₂, and CH₂CH₂S. In certain embodiments, heteroalkylene may also be optionally substituted with one or more substituents Q as described herein.

The term “alkenyl” refers to a linear or branched monovalent hydrocarbon radical, which contains one or more, in one embodiment, one, two, three, four, or five, in another embodiment, one, carbon-carbon double bond(s). The alkenyl may be optionally substituted with one or more substituents Q as described herein. The term “alkenyl” also embraces radicals having “cis” and “trans” configurations, or alternatively, “Z” and “E” configurations, as appreciated by those of ordinary skill in the art. As used herein, the term “alkenyl” encompasses both linear and branched alkenyl, unless otherwise specified. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkenylene” refers to a linear or branched divalent hydrocarbon radical, which contains one or more, in one embodiment, one, two, three, four, or five, in another embodiment, one, carbon-carbon double bond(s). The alkenylene may be optionally substituted with one or more substituents Q as described herein. Similarly, the term “alkenylene” also embraces radicals having “cis” and “trans” configurations, or alternatively, “E” and “Z” configurations. As used herein, the term “alkenylene” encompasses both linear and branched alkenylene, unless otherwise specified. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, allylene, propenylene, butenylene, and 4-methylbutenylene.

The term “heteroalkenylene” refers to a linear or branched divalent hydrocarbon radical, which contains one or more, in one embodiment, one, two, three, four, or five, in another embodiment, one, carbon-carbon double bond(s), and which contains one or more heteroatoms each independently selected from O, S, and N in the hydrocarbon chain. The heteroalkenylene may be optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, four, or five, in another embodiment, one, carbon-carbon triple bond(s). The alkynyl may be optionally substituted with one or more substituents Q as described herein. The term “alkynyl” also encompasses both linear and branched alkynyl, unless otherwise specified. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH) and propargyl (—CH₂C≡CH). For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms.

The term “cycloalkyl” refers to a cyclic saturated bridged and/or non-bridged monovalent hydrocarbon radical, which may be optionally substituted with one or more substituents Q as described herein. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclohexyl, cycloheptyl, bicyclo[2.1.1]hexyl, bicyclo[2.2.1]heptyl, decalinyl, and adamantyl.

The term “cycloalkenyl” refers to a cyclic unsaturated, nonaromatic bridged and/or non-bridged monovalent hydrocarbon radical, which may be optionally substituted with one or more substituents Q as described herein. In certain embodiments, the cycloalkenyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclobutenyl, cyclopentenyl, cyclohexenyl, or cycloheptenyl,

The term “aryl” refers to a monocyclic aromatic group and/or multicyclic monovalent aromatic group that contain at least one aromatic hydrocarbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may be optionally substituted with one or more substituents Q as described herein.

The terms “aralkyl” and “arylalkyl” refer to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 2-phenylethyl, and 3-phenylpropyl. In certain embodiments, the aralkyl are optionally substituted with one or more substituents Q as described herein.

The term “heteroaryl” refers to a monovalent monocyclic aromatic group or monovalent polycyclic aromatic group that contain at least one aromatic ring, wherein at least one aromatic ring contains one or more heteroatoms independently selected from O, S, N, and P in the ring. A heteroaryl group is bonded to the rest of a molecule through its aromatic ring. Each ring of a heteroaryl group can contain one or two 0 atoms, one or two S atoms, one to four N atoms, and/or one or two P atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, the heteroaryl may also be optionally substituted with one or more substituents Q as described herein as described herein.

The terms “heterocyclyl” and “heterocyclic” refer to a monovalent monocyclic non-aromatic ring system or monovalent polycyclic ring system that contains at least one non-aromatic ring, wherein one or more of the non-aromatic ring atoms are heteroatoms independently selected from O, S, N, and P; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. A heterocyclyl group is bonded to the rest of a molecule through its non-aromatic ring. In certain embodiments, the heterocyclyl is a monocyclic, bicyclic, tricyclic, or tetracyclic ring system, which may be spiro, fused, or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, β-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, the heterocyclyl may also be optionally substituted with one or more substituents Q as described herein.

The terms “halogen”, “halide” and “halo” refer to fluorine, chlorine, bromine, and/or iodine.

The term “optionally substituted” is intended to mean that a group or substituent, such as an alkyl, alkylene, heteroalkylene, alkenyl, alkenylene, heteroalkenylene, alkynyl, cycloalkyl, cycloalkenyl, aryl, aralkyl, heteroaryl, heteroaryl-C₁₋₆ alkyl, and heterocyclyl group, may be substituted with one or more substituents Q, each of which is independently selected from, e.g., (a) oxo (═O), halo, cyano (CN), and nitro (NO₂); (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, four, or five, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{g}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{a}C(O)R^{d}, —NR^{a}C(O)OR^{d}, —NR^{a}C(O)NR^{h}R^{c}, —NR^{a}C(═NR^{d})NR^{h}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)R^{a}R^{d}, —P(O)(OR^{a})R^{d}, —P(O)(OR^{d})(OR^{d}), —SR^{a}, —S(O)R^{a}, —S(O)₂R^{a}, —S(O)NR^{h}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four substituents Q^{a}; or (iii) R^{b} and R^{g} together with the N atom to which they are attached form heteroaryl or heterocyclyl, optionally substituted with one or more, in one embodiment, one, two, three, or four, substituents Q^{a}. As used herein, all groups that can be substituted are “optionally substituted,” unless otherwise specified.

In one embodiment, each substituent Q^{a} is independently selected from the group consisting of (a) oxo, cyano, halo, and nitro; and (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})R^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —P(O)R^{e}R^{h}, —P(O)(OR^{e})R^{h}, —P(O)(OR^{e})(OR^{h}), —S(O)R^{e}, —S(O)₂R^{e}, —S(O)NR^{f}R^{g}, and —S(O)₂NR^{f}R^{g}; wherein each Re, R^{f}, R^{g}, and R^{h} is independently (i) hydrogen, C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (ii) R^{f} and R^{g} together with the N atom to which they are attached form heteroaryl or heterocyclyl.

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The phrase “an enantiomer, a mixture of enantiomers, a mixture of two or more diastereomers, or an isotopic variant thereof; or a pharmaceutically acceptable salt, solvate, hydrate, or prodrug thereof” has the same meaning as the phrase “an enantiomer, a mixture of enantiomers, a mixture of two or more diastereomers, or an isotopic variant of the compound referenced therein; or a pharmaceutically acceptable salt, solvate, hydrate, or prodrug of the compound referenced therein; or a pharmaceutically acceptable salt, solvate, hydrate, or prodrug of an enantiomer, a mixture of enantiomers, a mixture of two or more diastereomers, or an isotopic variant of the compound referenced therein.”

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

Resistant, relapsed or refractory refers to when a cancer that has a reduced responsiveness to a treatment, e.g., up to the point where the cancer does not respond to treatment. The cancer can be resistant at the beginning of treatment, or it may become resistant during treatment. The term “refractory” can refer to a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

The terms “responsiveness” or “to respond” to treatment, and other forms of this term, as used herein, refer to the reaction of a subject to treatment with a therapeutic, e.g., a PI3K inhibitor, alone or in combination, e.g., monotherapy or combination therapy. Responsiveness to a therapy, e.g., treatment with a PI3K inhibitor alone or in combination, can be evaluated by comparing a subject's response to the therapy using one or more clinical criteria, such as IWCLL 2008 (for CLL) described in, e.g., Hallek, M. et al. (2008) Blood 111 (12): 5446-5456; the Lugano Classification described in, e.g., Cheson, B. D. et al. Journal of Clinical Oncology, 32(27): 3059-3067; and the like. Additional classifications of responsiveness are provided by. These criteria provide a set of published rules that define when cancer patients improve (“respond”), stay the same (“stable”) or worsen (“progression”) during treatments.

For example, a subject having CLL can be determined to be in complete remission (CR) or partial remission (PR). For example, according to IWCLL 2008, a subject is considered to be in CR if at least all of the following criteria as assessed after completion of therapy are met: (i) Peripheral blood lymphocytes (evaluated by blood and different count) below 4×10⁹/L (4000 μi); (ii) no hepatomegaly or splenomegaly by physical examination; (iii) absence of constitutional symptoms; and (iv) blood counts (e.g., neutrophils, platelets, hemoglobin) above the values set forth in Hallek, M. et al. Partial remission (PR) for CLL is defined according to IWCLL 2008 as including one of: (i) a decrease in number of blood lymphocytes by 50% or more from the value before therapy; (ii) a reduction in lymphadenopathy, as detected by CT scan or palpation; or (iii) a reduction in pretreatment enlargement of spleen or liver by 50% or more, as detected by CT scan or palpation; and blood counts (e.g., neutrophils, platelets, hemoglobin) according to the values set forth in Hallek, M. et al. In other embodiments, a subject having CLL is determined to have progressive disease (PD) or stable disease (SD). For example, according to IWCLL 2008, a subject is considered to be in PD during therapy or after therapy if at least one of the following criteria is met: (i) progression on lymphadenopathy; (ii) an increase in pretreatment enlargement of spleen or liver by 50% or more, or de novo appearance of hepatomegaly or splenomegaly; (iii) an increase in the number of blood lymphocytes by 50% or more with at least 5000 B lymphocytes per microliter; (iv) transformation to a more aggressive histology (e.g., Richter syndrome); or (v) occurrence of cytopenia (neutropenia, anemia or thrombocytopenia) attributable to CLL. Stable disease (SD) for CLL is defined according to IWCLL 2008 as a patient who has not achieved CR or a PR, and who has not exhibited progressive disease.

For example, in some embodiments, a subject with CLL responds to treatment with an PI3K inhibitor, alone or in combination, if at least one of the criteria for disease progression according to IWCLL is retarded or reduced, e.g., by about 10%, 20%, 30%, 40%, 50%, 60%, 70%, 80%, 90% or more. In another example, a subject responds to treatment with a PI3K inhibitor, alone or in combination, if the subject experiences a life expectancy extension, e.g., extended by about 5%, 10%, 20%, 30%, 40%, 50% or more beyond the life expectancy predicted if no treatment is administered. In another example, a subject responds to treatment with a PI3K inhibitor, alone or in combination, if the subject has one or more of: an increased progression-free survival, overall survival or increased time to progression (TTP), e.g., as described in Hallek, M. et al.

### Compounds

Disclosed herein are PI3K inhibitors of Formula (I):

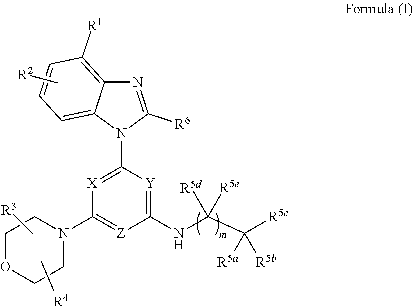

or an enantiomer, a mixture of enantiomers, a mixture of two or more diastereomers, or an isotopic variant thereof; or a pharmaceutically acceptable salt, solvate, hydrate, or prodrug thereof; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —C(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁵ is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1d}, R^{5a}, R^{5b}, R^{5c}, R^{5d}, R^{5e}, R^{5f} and R^{5g} is optionally substituted with one or more, in one embodiment, one, two, three, four, or five substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{h}R^{c}, —OS(O)R^{a}, —OS(O)₂R^{a}, —OS(O)NR^{h}R^{c}, —OS(O)₂NR^{h}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{h}R^{c}, —SR^{a}, —S(O)R^{a}, —S(O)₂R^{a}, —S(O)NR^{h}R^{c}, and —S(O)₂NR^{h}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (iii) R^{b} and R^{c} together with the N atom to which they are attached form heterocyclyl, which is further optionally substituted with one or more, in one embodiment, one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR⁶)NR^{f}R^{g}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl; or
wherein two substituents Q that are adjacent to each other optionally form a C₃₋₁₀ cycloalkenyl, C₆₋₁₄ aryl, heteroaryl, or heterocyclyl, each optionally substituted with one, two, three, or four substituents Q^{a}.

In one embodiment of a compound of Formula (I),
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R⁵ is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d},—NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl is optionally substituted with one or more, in one embodiment, one, two, three, four, or five substituents Q as defined herein.

In another embodiment of a compound of Formula (I),
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; wherein each R^{1a}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d},—NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R⁵%-(C₆₋₁₄ aryl) or —(CR^{5f}R⁵%-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl is optionally substituted with one or more, in one embodiment, one, two, three, four, or five substituents Q as defined herein.

In yet another embodiment of a compound of Formula (I),
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1d}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1d}, —S(O)R^{1d}, —S(O)₂R^{1a}, —S(O)NR^{1b}R^{1c}, or —S(O)₂NR^{1b}R^{1c}; wherein each R^{1d}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1d}, —OC(O)R^{1d}, —OC(O)OR^{1d}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ-(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl is optionally substituted with one or more, in one embodiment, one, two, three, four, or five substituents Q as defined herein.

In still another embodiment of a compound of Formula (I),
X, Y, and Z are N;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1d}, —C(O)NR^{1b}R^{1c}, —C(NR^{1a})NR^{1b}R^{1c}, —OR^{1d}, —OC(O)R^{1d}, —OC(O)OR^{1d}, —OC(O)NR^{1b}R^{1c}, —OC(═NR^{1a})NR^{1b}R^{1c}, —OS(O)R^{1a}, —OS(O)₂R^{1d}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1d}, —S(O)R^{1d}, —S(O)₂R^{1a}, —S(O)NR^{1b}R^{1c}, or —S(O)₂NR^{1b}R^{1c}; wherein each R^{1d}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1d}, —C(O)OR^{1d}, —C(O)NR^{1b}R^{1c}, —C(NR^{1a})NR^{1b}R^{1c}, —OC(O)R^{1d}, —OC(O)OR^{1a}, —OC(O)NR^{1b}R^{1c}, —OC(═NR^{1a})NR^{1b}R^{1c}, —OS(O)R^{1a}, —OS(O)₂R^{1d}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1d}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1d}, —OC(O)NR^{1b}R^{1c}, —OC(═NR^{1a})NR^{1b}R^{1c}, —OS(O)R^{1a}, —OS(O)₂R^{1d}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR¹)NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5c} is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl is optionally substituted with one or more, in one embodiment, one, two, three, four, or five substituents Q as defined herein.

Synthesis of compounds of Formula (I) is described in U.S. Pat. No. 9,056,852 B2, which is incorporated by reference for such disclosure.

Also provided herein is a compound of Formula (IX):

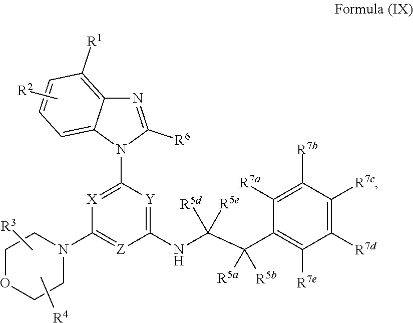

or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7e}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or
two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}; and
R¹, R², R³, R⁴, R⁶, R^{1a}, R^{1b}, R^{1c}, R^{1d}, R^{5a}, R^{5b}, R^{5d}, R^{5e}, X, Y, and Z are each as defined herein.

Synthesis of compounds Formula (IX) is described in U.S. Pat. No. 9,056,852 B2, which is incorporated by reference for such disclosure.

In one embodiment, the compound of Formula (IX) has the structure of Formula (IXa):

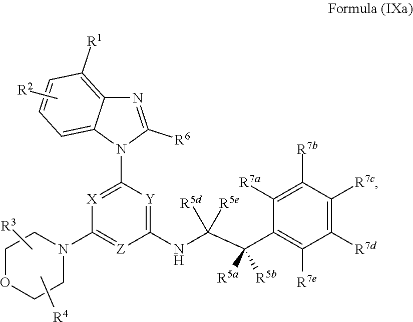

or an isotopic variant thereof; or a pharmaceutically acceptable salt, solvate, hydrate, or prodrug thereof; wherein R¹, R², R³, R⁴, R⁶, R^{5a}, R^{5b}, R^{5d}, R^{5e}, R^{7a}, R^{7b}, R^{7c}, R^{7d}, R^{7e}, X, Y, and Z are each as defined herein.

Synthesis of compounds of Formula (IXa) is described in U.S. Pat. No. 9,056,852 B2, which is incorporated by reference for such disclosure.

In another embodiment, the compound of Formula (IX) has the structure of Formula (IXb):

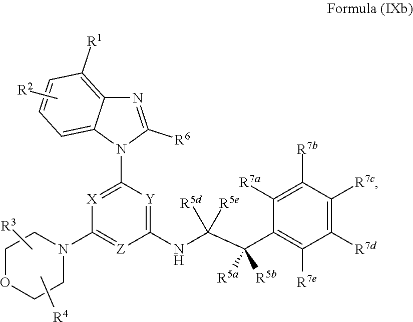

or an isotopic variant thereof; or a pharmaceutically acceptable salt, solvate, hydrate, or prodrug thereof; wherein R¹, R², R³, R⁴, R⁶, R^{5a}, R^{5b}, R^{5d}, R^{5e}, R^{7a}, R^{7b}, R^{7c}, R^{7d}, R^{7e}, X, Y, and Z are each as defined herein.

Synthesis of compounds of Formula (IXb) is described in U.S. Pat. No. 9,056,852 B2, which is incorporated by reference for such disclosure.

In certain embodiments of compounds of Formula (IX), (IXa), or (IXb), one of R^{7a}, R^{7b}, R^{7c}, R^{7a}, and R^{7e} is C₆₋₁₄ aryl, heteroaryl, or heterocyclyl, each of which is optionally substituted with one, two, three, or four substituents Q^{a}; in certain embodiments, one of R^{7a}, R^{7b}, R^{7c}, R^{7d}, and R^{7e} is C₆₋₁₄ aryl, e.g., phenyl, optionally substituted with one, two, three, or four substituents Q^{a}; in certain embodiments, one of R^{7a}, R^{7b}, R^{7c}, R^{7a}, and R^{7e} is heteroaryl, e.g., 5-membered or 6-membered heteroaryl, optionally substituted with one, two, three, or four substituents Q^{a}; in certain embodiments, one of R^{7a}, R^{7b}, R^{7c}, R^{7d}, and R^{7e} is heterocyclyl, e.g., 5-membered or 6-membered heterocyclyl, optionally substituted with one, two, three, or four substituents Q^{a}; in certain embodiments, one of R^{7a}, R^{7b}, R^{7c}, R^{7a}, and R^{7e} is phenyl, imidazolyl, pyrozolyl, pyridinyl, piperidinyl, or piperazinyl, each optionally substituted with one, two, three, or four substituents Q^{a}; in certain embodiments, one of R^{7a}, R^{7b}, R^{7c}, R^{7a}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, one of R^{7a}, R^{7b}, R^{7c}, R^{7a}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, one of R^{7a}, R^{7b}, R^{7c}, R^{7a}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments of compounds of Formula (IX), (IXa), or (IXb), is C₆₋₁₄ aryl, heteroaryl, or heterocyclyl, each of which is optionally substituted with one, two, three, or four substituents Q^{a}; in certain embodiments, R^{7a} is C₆₋₁₄ aryl, e.g., phenyl, optionally substituted with one, two, three, or four substituents Q^{a}; in certain embodiments, R^{7a} is heteroaryl, e.g., 5-membered or 6-membered heteroaryl, optionally substituted with one, two, three, or four substituents Q^{a}; in certain embodiments, R^{7a} is heterocyclyl, e.g., 5-membered or 6-membered heterocyclyl, optionally substituted with one, two, three, or four substituents Q^{a}; in certain embodiments, R^{7a} is phenyl, imidazolyl, pyrozolyl, pyridinyl, piperidinyl, or piperazinyl, each optionally substituted with one, two, three, or four substituents Q^{a}; in certain embodiments, R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments of compounds of Formula (IX), (IXa), or (IXb),

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently hydrogen, halo, C₁₋₆ alkyl, optionally substituted with one, two, three, four, or five substituents Q;

R^{5d} and R^{5e} are each independently C₁₋₆ alkyl, optionally substituted with one, two, three, four, or five substituents Q;

R^{7a} is C₆₋₁₄ aryl, heteroaryl, or heterocyclyl, each of which is optionally substituted with one, two, three, or four substituents Q^{a};

R^{7c}, R^{7d}, and R^{7e} are hydrogen; and

X, Y, and Z are each independently N or CR^{x}, with the proviso that at least two of X, Y, and Z are N; where R^{x} is a hydrogen or C₁₋₆ alkyl, optionally substituted with one, two, three, or four substituents Q^{a}.

In certain embodiments of compounds of Formula (IX), (IXa), or (IXb),

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are hydrogen;

R^{5d} and R^{5e} are each independently C₁₋₆ alkyl;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (IX), (IXa), or (IXb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;
R^{7a} is C₆₋₁₄ aryl, monocyclic heteroaryl, or monocyclic heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (IX), (IXa), or (IXb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is phenyl, 5- or 6-membered heteroaryl, or 5- or 6-membered heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (IX), (IXa), or (IXb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (IX), (IXa), or (IXb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is phenyl, imidazolyl, pyrozolyl, pyridinyl, piperidinyl, or piperazinyl, each of which is optionally substituted with one, two, three, or four substituents Q^{a};

R^{7b}, R^{7c}, R^{7d}, and R^{7e} are hydrogen; and

X, Y, and Z are each independently N or CH.

Also provided herein is a compound of Formula (X):

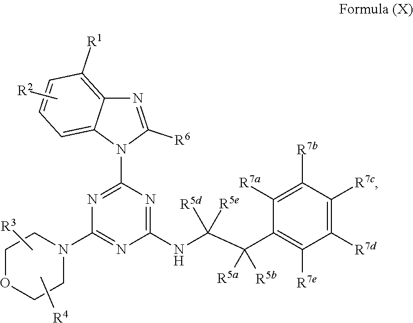

or an enantiomer, a mixture of enantiomers, a mixture of two or more diastereomers, or an isotopic variant thereof; or a pharmaceutically acceptable salt, solvate, hydrate, or prodrug thereof; wherein R¹, R², R³, R⁴, R⁶, R^{5a}, R^{5b}, R^{5d}, R^{5e}, R^{7a}, R^{7b}, R^{7c}, R^{7d}, and R^{7e} are each as defined herein.

Synthesis of compounds of Formula (X) is described in U.S. Pat. No. 9,056,852 B2, which is incorporated by reference for such disclosure.

In one embodiment, the compound of Formula (X) has the structure of Formula (Xa):

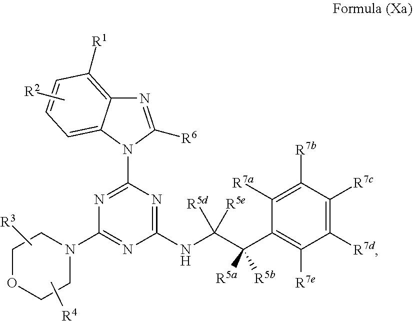

or an isotopic variant thereof; or a pharmaceutically acceptable salt, solvate, hydrate, or prodrug thereof; wherein R¹, R², R³, R⁴, R⁶, R^{5a}, R^{5b}, R^{5d}, R^{5e}, R^{7a}, R^{7b}, R^{7c}, R^{7a}, and R^{7e} are each as defined herein.

Synthesis of compounds of Formula (Xa) is described in U.S. Pat. No. 9,056,852 B2, which is incorporated by reference for such disclosure.

In another embodiment, the compound of Formula (X) has the structure of Formula (Xb):

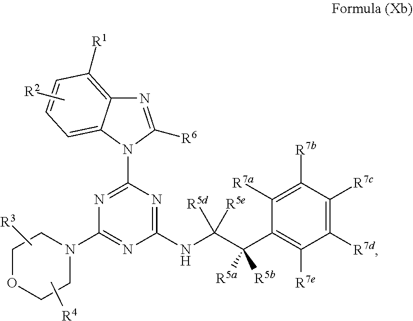

or an isotopic variant thereof; or a pharmaceutically acceptable salt, solvate, hydrate, or prodrug thereof; wherein R¹, R², R³, R⁴, R⁶, R^{5a}, R^{5b}, R^{5d}, R^{5e}, R^{7a}, R^{7b}, R^{7c}, R^{7a}, and R^{7e} are each as defined herein.

Synthesis of compounds of Formula (Xb) is described in U.S. Pat. No. 9,056,852 B2, which is incorporated by reference for such disclosure.

In certain embodiments of compounds of Formula (X), (Xa), or (Xb), one of R^{7a}, R^{7b}, R^{7c}, R^{7a}, and R^{7e} is C₆₋₁₄ aryl, heteroaryl, or heterocyclyl, each of which is optionally substituted with one, two, three, or four substituents Q^{a}; in certain embodiments, one of R^{7a}, R^{7b}, R^{7c}, R^{7d}, and R^{7e} is C₆₋₁₄ aryl, e.g., phenyl, optionally substituted with one, two, three, or four substituents Q^{a}; in certain embodiments, one of R^{7a}, R^{7b}, R^{7c}, R^{7a}, and R^{7e} is heteroaryl, e.g., 5-membered or 6-membered heteroaryl, optionally substituted with one, two, three, or four substituents Q^{a}; in certain embodiments, one of R^{7a}, R^{7b}, R^{7c}, R^{7d}, and R^{7e} is heterocyclyl, e.g., 5-membered or 6-membered heterocyclyl, optionally substituted with one, two, three, or four substituents Q^{a}; in certain embodiments, one of R^{7a}, R^{7b}, R^{7c}, R^{7a}, and R^{7e} is phenyl, imidazolyl, pyrozolyl, pyridinyl, piperidinyl, or piperazinyl, each optionally substituted with one, two, three, or four substituents Q^{a}; in certain embodiments, one of R^{7a}, R^{7b}, R^{7c}, R^{7a}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, one of R^{7a}, R^{7b}, R^{7c}, R^{7a}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, one of R^{7a}, R^{7b}, R^{7c}, R^{7a}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments of compounds of Formula (X), (Xa), or (Xb), R^{7a} is C₆₋₁₄ aryl, heteroaryl, or heterocyclyl, each of which is optionally substituted with one or more substituents Q^{a}; in certain embodiments, R^{7a} is C₆₋₁₄ aryl, e.g., phenyl, optionally substituted with one, two, three, or four substituents Q^{a}; in certain embodiments, R^{7a} is heteroaryl, e.g., 5-membered or 6-membered heteroaryl, optionally substituted with one, two, three, or four substituents Q^{a}; in certain embodiments, R^{7a} is heterocyclyl, e.g., 5-membered or 6-membered heterocyclyl, optionally substituted with one, two, three, or four substituents Q^{a}; in certain embodiments, R^{7a} is phenyl, imidazolyl, pyrozolyl, pyridinyl, piperidinyl, or piperazinyl, each optionally substituted with one, two, three, or four substituents Q^{a}; in certain embodiments, R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments of compounds of Formula (X), (Xa), or (Xb),

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently hydrogen, halo, C₁₋₆ alkyl, optionally substituted with one, two, three, four, or five substituents Q;

R^{5d} and R^{5e} are each independently C₁₋₆ alkyl, optionally substituted with one, two, three, four, or five substituents Q;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In certain embodiments of compounds of Formula (X), (Xa), or (Xb),

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are hydrogen;

R^{5d} and R^{5e} are each independently C₁₋₆ alkyl;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In certain embodiments of compounds of Formula (X), (Xa), or (Xb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is C₆₋₁₄ aryl, monocyclic heteroaryl, or monocyclic heterocyclyl, each of which is optionally substituted with one, two, three, or four substituents Q^{a}; and

R^{7b}, R^{7c}, R^{7d}, and R^{7e} are hydrogen.

In certain embodiments of compounds of Formula (X), (Xa), or (Xb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is phenyl, 5- or 6-membered heteroaryl, or 5- or 6-membered heterocyclyl, each of which is optionally substituted with one, two, three, or four substituents Q^{a}; and

R^{7b}, R^{7c}, R^{7d}, and R^{7e} are hydrogen.

In certain embodiments of compounds of Formula (X), (Xa), or (Xb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a}; and

R^{7b}, R^{7c}, R^{7d}, and R^{7e} are hydrogen.

In certain embodiments of compounds of Formula (X), (Xa), or (Xb),

R¹ is hydrogen or methoxy;

R² is hydrogen;

R³ and R⁴ are hydrogen;

R⁶ is difluoromethyl;

R^{5a} and R^{5b} are hydrogen;

R^{5d} and R^{5e} are methyl;

R^{7a} is phenyl, imidazolyl, pyrozolyl, pyridinyl, piperidinyl, or piperazinyl, each of which is optionally substituted with one, two, three, or four substituents Q^{a}; and

R^{7b}, R^{7c}, R^{7d}, and R^{7e} are hydrogen.

Provided herein is a compound of Formula (XI):

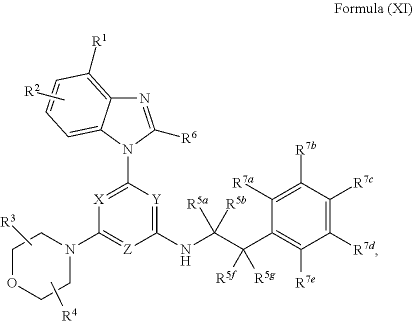

or an enantiomer, a mixture of enantiomers, a mixture of two or more diastereomers, or an isotopic variant thereof; or a pharmaceutically acceptable salt, solvate, hydrate, or prodrug thereof; wherein: R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —S(O)R^{a}, —OS(O)₂R^{a}, —S(O)NR^{b}R^{c}, —S(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}; and
   R¹, R², R³, R⁴, R⁶, R^{5a}, R^{5b}, R^{5f}, R^{5g}, X, Y, and Z are each as defined herein.

Synthesis of compounds of Formula (XI) is described in U.S. Pat. No. 9,056,852 B2, which is incorporated by reference for such disclosure.

In one embodiment, the compound of Formula (XI) has the structure of Formula (XIa):

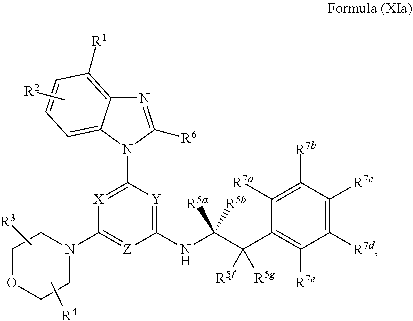

or an isotopic variant thereof; or a pharmaceutically acceptable salt, solvate, hydrate, or prodrug thereof; wherein R¹, R², R³, R⁴, R⁶, R^{5a}, R^{5b}, R^{5f}, R^{5g}, R^{7a}, R^{7b}, R^{7c}, R^{7d}, R^{7e}, X, Y, and Z are each as defined herein.

Synthesis of compounds of Formula (XIa) is described in U.S. Pat. No. 9,056,852 B2, which is incorporated by reference for such disclosure.

In another embodiment, the compound of Formula (XI) has the structure of Formula (XIb):

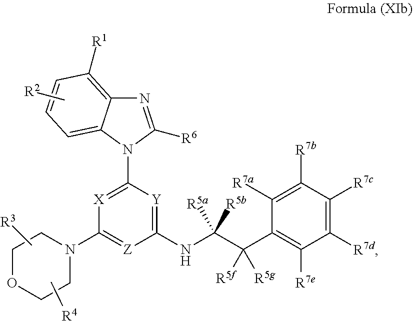

or an isotopic variant thereof; or a pharmaceutically acceptable salt, solvate, hydrate, or prodrug thereof; wherein R¹, R², R³, R⁴, R⁶, R^{5a}, R^{5b}, R^{5f}, R^{5g}, R^{7a}, R^{7b}, R^{7c}, R^{7d}, R^{7e}, X, Y, and Z are each as defined herein.

Synthesis of compounds of Formula (XIb) is described in U.S. Pat. No. 9,056,852 B2, which is incorporated by reference for such disclosure.

In certain embodiments of compound of Formula (XI), (XIa), or (XIb), R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; and R¹, R², R³, R⁴, R⁵, R^{5g}, R⁶, R^{7a}, R^{7c}, R^{7d}, R^{7e}, X, Y, Z, R^{1a}, R^{1b}, R^{1c}, and R^{1d} are defined herein elsewhere.

In certain embodiments of compounds of Formula (XI), (XIa), or (XIb), one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; in certain embodiments, one of R^{7a}, R^{7b}, R^{7c}, R^{7d}, and R^{7e} is C₆₋₁₄ aryl, e.g., phenyl, optionally substituted with one, two, three, or four substituents Q^{a}; in certain embodiments, one of R^{7a}, R^{7b}, R^{7c}, R^{7d}, and R^{7e} is heteroaryl, e.g., 5-membered or 6-membered heteroaryl, optionally substituted with one, two, three, or four substituents Q^{a}; in certain embodiments, one of R^{7a}, R^{7b}, R^{7c}, R^{7d}, and R^{7e} is heterocyclyl, e.g., 5-membered or 6-membered heterocyclyl, optionally substituted with one, two, three, or four substituents Q^{a}; in certain embodiments, one of R^{7a}, R^{7b}, R^{7c}, R^{7d}, and R^{7e} is phenyl, imidazolyl, pyrozolyl, pyridinyl, piperidinyl, or piperazinyl, each optionally substituted with one, two, three, or four substituents Q^{a}; in certain embodiments, one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one or more substituents Q^{a}; in certain embodiments, one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments of compounds of Formula (XI), (XIa), or (XIb), R^{7a} is C₆₋₁₄ aryl, heteroaryl, or heterocyclyl, each of which is optionally substituted with one, two, three, or four substituents Q^{a}; in certain embodiments, R^{7a} is C₆₋₁₄ aryl, e.g., phenyl, optionally substituted with one, two, three, or four substituents Q^{a}; in certain embodiments, R^{7a} is heteroaryl, e.g., 5-membered or 6-membered heteroaryl, optionally substituted with one, two, three, or four substituents Q^{a}; in certain embodiments, R^{7a} is heterocyclyl, e.g., 5-membered or 6-membered heterocyclyl, optionally substituted with one, two, three, or four substituents Q^{a}; in certain embodiments, R^{7a} is phenyl, imidazolyl, pyrozolyl, pyridinyl, piperidinyl, or piperazinyl, each optionally substituted with one, two, three, or four substituents Q^{a}; in certain embodiments, R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; in certain embodiments, R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and in certain embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments of compounds of Formula (XI), (XIa), or (XIb),

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently C₁₋₆ alkyl, optionally substituted with one, two, three, four, or five substituents Q;

R^{5f} and R^{5g} are each independently hydrogen, halo, C₁₋₆ alkyl, optionally substituted with one, two, three, four, or five substituents Q; or R^{5f} and R^{5g} together with the carbon atom to which they are attached form C₁₋₁₀ cycloalkyl or heterocyclyl, each of which is optionally substituted with one, two, three, four, or five substituents Q;

R^{7a} is C₆₋₁₄ aryl, heteroaryl, or heterocyclyl, each of which is optionally substituted with one, two, three, or four substituents Q^{a};

R^{7c}, R^{7d}, and R^{7e} are hydrogen; and

X, Y, and Z are each independently N or CR^{x}, with the proviso that at least two of X, Y, and Z are N; where R^{x} is a hydrogen or C₁₋₆ alkyl, optionally substituted with one, two, three, or four substituents Q^{a}.

In certain embodiments of compounds of Formula (XI), (XIa), or (XIb),

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently C₁₋₆ alkyl;

R^{5f} and R^{5g} are each independently hydrogen or C₁₋₆ alkyl; or R^{5f} and R^{5g} together with the carbon atom to which they are attached form C₁₋₁₀ cycloalkyl;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (XI), (XIa), or (XIb),

R¹ is hydrogen or methoxy;

R² is hydrogen;

R³ and R⁴ are hydrogen;

R⁶ is difluoromethyl;

R^{5a} and R^{5b} are methyl;

R^{5f} and R^{5g} are hydrogen; or R^{5f} and R^{5g} together with the carbon atom to which they are attached form cyclopropyl, cyclobutyl, cyclopentyl, or cyclohexyl;

R^{7a} is C₆₋₁₄ aryl, monocyclic heteroaryl, or monocyclic heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (XI), (XIa), or (XIb),

R¹ is hydrogen or methoxy;

R² is hydrogen;

R³ and R⁴ are hydrogen;

R⁶ is difluoromethyl;

R^{5a} and R^{5b} are methyl;

R^{5f} and R^{5g} are hydrogen; or R^{5f} and R^{5g} together with the carbon atom to which they are attached form cyclopropyl, cyclobutyl, cyclopentyl, or cyclohexyl;

R^{7a} is phenyl, 5- or 6-membered heteroaryl, or 5- or 6-membered heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In certain embodiments of compounds of Formula (XI), (XIa), or (XIb),

R¹ is hydrogen or methoxy;

R² is hydrogen;

R³ and R⁴ are hydrogen;

R⁶ is difluoromethyl;

R^{5a} and R^{5b} are methyl;

R^{5f} and R^{e}g are hydrogen; or R^{5f} and R^{5g} together with the carbon atom to which they are attached form cyclopropyl, cyclobutyl, cyclopentyl, or cyclohexyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};

R^{7b}, R^{7c}, R^{7d}, and R^{7g} are hydrogen; and

X, Y, and Z are each independently N or CH.

In certain embodiments of compounds of Formula (XI), (XIa), or (XIb),

R¹ is hydrogen or methoxy;

R² is hydrogen;

R³ and R⁴ are hydrogen;

R⁶ is difluoromethyl;

R^{5a} and R^{5b} are methyl;

R^{5f} and R^{e}g are hydrogen; or R^{5f} and R^{5g} together with the carbon atom to which they are attached form cyclopropyl, cyclobutyl, cyclopentyl, or cyclohexyl;

R^{7a} is phenyl, imidazolyl, pyrozolyl, pyridinyl, piperidinyl, or piperazinyl, each of which is optionally substituted with one, two, three, or four substituents Q^{a};

R^{7b}, R^{7c}, R^{7d}, and R^{7g} are hydrogen; and

X, Y, and Z are each independently N or CH.

Provided herein is a compounds of Formula (XVI):

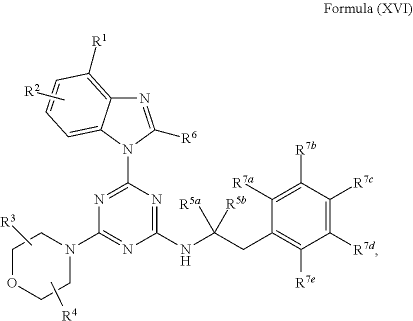

or an enantiomer, a mixture of enantiomers, a mixture of two or more diastereomers, or an isotopic variant thereof; or a pharmaceutically acceptable salt, solvate, hydrate, or prodrug thereof; wherein R¹, R², R³, R⁴, R⁶, R^{5a}, R^{5b}, R^{7a}, R^{7b}, R^{7c}, R^{7d}, and R^{7e} are each as defined herein.

Synthesis of compounds of Formula (XVI) is described in U.S. Pat. No. 9,056,852 B2, which is incorporated by reference for such disclosure.

In one embodiment of a compound of Formula (XVI), one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In another embodiment of a compound of Formula (XVI), one of R^{7a}, R^{7b}, R^{7c}, R^{7d}, and R^{7e} is C₆₋₁₄ aryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is 5-membered or 6-membered heteroaryl, which is optionally substituted with one, two, three, or four substituents Q^{a}; and R¹, R², R³, R⁴, R⁶, R^{5a}, R^{5b}, K the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is 5-membered or 6-membered heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, imidazolyl, pyrozolyl, pyridinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In yet another embodiment of a compound of Formula (XVI), one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In still another embodiment of a compound of Formula (XVI), one of R^{7a}, R^{7b}, R^{7c}, R^{7a}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and R¹, R², R³, R⁴, R⁶, R^{5a}, R^{5b}, the remaining of R^{7a}, R^{7b}, R^{7c}, R^{7a}, and R^{7e}, X, Y, and Z are each as defined herein.

In one embodiment of a compound of Formula (XVI), R^{7a} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In another embodiment of a compound of Formula (XVI), R^{7a} is C₆₋₁₄ aryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is heteroaryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is 5-membered or 6-membered heteroaryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is 5-membered or 6-membered heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is phenyl, imidazolyl, pyrozolyl, pyridinyl, piperidinyl, or piperazinyl, each optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In yet another embodiment of a compound of Formula (XVI), R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In yet another embodiment of a compound of Formula (XVI), R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVI),

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently C₁₋₆ alkyl, optionally substituted with one, two, three, four, or five substituents Q;

R^{7a} is C₆₋₁₄ aryl, heteroaryl, or heterocyclyl, each of which is optionally substituted with one or more substituents Q^{a}; and

R^{7b}, R^{7c}, R^{7d}, and R^{7e} are hydrogen.

In one embodiment of a compound of Formula (XVI),

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently C₁₋₆ alkyl;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVI),

R¹ is hydrogen or methoxy;

R² is hydrogen;

R³ and R⁴ are hydrogen;

R⁶ is difluoromethyl;

R^{5a} and R^{5b} are methyl;

R^{7a} is C₆₋₁₄ aryl, monocyclic heteroaryl, or monocyclic heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVI),

R¹ is hydrogen or methoxy;

R² is hydrogen;

R³ and R⁴ are hydrogen;

R⁶ is difluoromethyl;

R^{5a} and R^{5b} are methyl;

R^{7a} is phenyl, 5- or 6-membered heteroaryl, or 5- or 6-membered heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVI),

R¹ is hydrogen or methoxy;

R² is hydrogen;

R³ and R⁴ are hydrogen;

R⁶ is difluoromethyl;

R^{5a} and R^{5b} are methyl;

R^{7a} is phenyl, imidazolyl, pyrozolyl, pyridinyl, pyrimidinyl, pyrrolidinyl, piperidinyl, or piperazinyl, each of which is optionally substituted with one, two, three, four, or five substituents Q; and

R^{7b}, R^{7c}, R^{7d}, and R^{7e} are hydrogen.

In one embodiment of a compound of Formula (XVI),

R¹ is hydrogen or methoxy;

R² is hydrogen;

R³ and R⁴ are hydrogen;

R⁶ is difluoromethyl;

R^{5a} and R^{5b} are methyl;

R^{7a} is phenyl, imidazolyl, pyrozolyl, pyridinyl, piperidinyl, or piperazinyl, each of which is optionally substituted with one, two, three, or four substituents Q^{a}; and

R^{7b}, R^{7c}, R^{7d}, and R^{7e} are hydrogen.

In one embodiment, the compound of Formula (XVI) has the structure of Formula (XVIa):

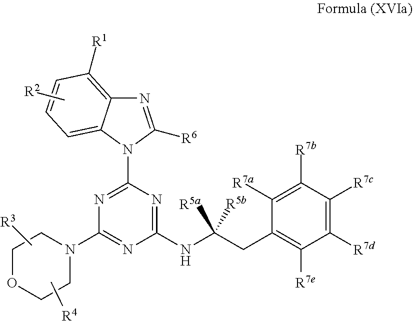

or an isotopic variant thereof; or a pharmaceutically acceptable salt, solvate, hydrate, or prodrug thereof; wherein R¹, R², R³, R⁴, R⁶, R^{5a}, R^{5b}, R^{7a}, R^{7b}, R^{7c}, and R^{7e} are each as defined herein.

Synthesis of compounds of Formula (XVIa) is described in U.S. Pat. No. 9,056,852 B2, which is incorporated by reference for such disclosure.

In one embodiment of a compound of Formula (XVIa), one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is C₆₋₁₄ aryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is 5-membered or 6-membered heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is 5-membered or 6-membered heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is phenyl, imidazolyl, pyrozolyl, pyridinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In one embodiment of a compound of Formula (XVIa), one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIa), R^{7a} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa), R^{7a} is C₆₋₁₄ aryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa), R^{7a} is heteroaryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa), R^{7a} is 5-membered or 6-membered heteroaryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa), R^{7a} is heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, X, Y, and Z are each as defined herein.

In one embodiment of a compound of Formula (XVIa), R^{7a} is 5-membered or 6-membered heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa), R^{7a} is phenyl, imidazolyl, pyrozolyl, pyridinyl, piperidinyl, or piperazinyl, each optionally substituted with one, two, three, or four substituents Q. and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa), R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, R^{7c}, R^{7d}, R^{7e}, X, Y, and Z are each as defined herein.

In one embodiment of a compound of Formula (XVIa), R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In one embodiment of a compound of Formula (XVIa), R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIa),

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently hydrogen or C₁₋₆ alkyl, optionally substituted with one, two, three, four, or five substituents Q;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIa),

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently hydrogen or C₁₋₆ alkyl;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIa),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is C₆₋₁₄ aryl, monocyclic heteroaryl, or monocyclic heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIa),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, 5- or 6-membered heteroaryl, or 5- or 6-membered heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIa),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, pyrrolidinyl, piperidinyl, piperidinyl, or piperazinyl, each of which is optionally substituted with one, two, three, or four substituents Q^{a}; and

R^{7b}, R^{7c}, R^{7d}, and R^{7e} are hydrogen.

In one embodiment of a compound of Formula (XVIa),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, piperidinyl, or piperazinyl, each of which is optionally substituted with one, two, three, or four substituents Q^{a}; and

R^{7b}, R^{7c}, R^{7d}, and R^{7e} are hydrogen.

Synthesis of compounds of Formula (XVIa) is described in U.S. Pat. No. 9,056,852 B2, which is incorporated by reference for such disclosure.

In another embodiment, the compound of Formula (XVI) has the structure of Formula (XVIb):

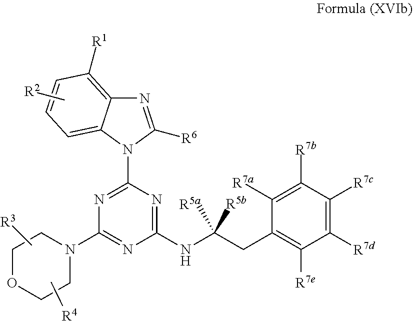

or an isotopic variant thereof; or a pharmaceutically acceptable salt, solvate, hydrate, or prodrug thereof; wherein R¹, R², R³, R⁴, R⁶, R^{5a}, R^{5b}, R^{7a}, R^{7b}, R^{7c}, and R^{7e} are each as defined herein.

In one embodiment of a compound of Formula (XVIb), one of R^{7a}, R^{7b}, R^{7c}, R^{7d}, and R^{7e} is C₆₋₁₄ aryl, heteroaryl, or heterocyclyl, each of which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is C₆₋₁₄ aryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is 5-membered or 6-membered heteroaryl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is 5-membered or 6-membered heterocyclyl, which is optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is phenyl, imidazolyl, pyrozolyl, pyridinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), one of R^{7a}, R^{7b}, R^{7c}, R^{7d}, and R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In one embodiment of a compound of Formula (XVIb), one of R^{7a}, R^{7b}, R^{7c}, R^{7d}, and R^{7e} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and R¹, R², R³, R⁴, R⁶, R^{5a}, R^{5b}, the remaining of R^{7a}, R^{7b}, R^{7c}, R^{7d}, and R^{7e}, X, Y, and Z are each as defined herein.

In one embodiment of a compound of Formula (XVIb), is C₆₋₁₄ aryl, heteroaryl, or heterocyclyl, each of which is optionally substituted with one, two, three, four, or five substituents Q; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is C₆₋₁₄ aryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is heteroaryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is 5-membered or 6-membered heteroaryl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is heterocyclyl, which is optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is 5-membered or 6-membered heterocyclyl, which is optionally substituted with one, two, three, or four substituents Q^{a}; and

R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is phenyl, imidazolyl, pyrozolyl, pyridinyl, piperidinyl, or piperazinyl, each optionally substituted with one, two, three, or four substituents Q. and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb), R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In one embodiment of a compound of Formula (XVIb), R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-methylpyridin-4-yl, 2-methoxypyridin-4-yl, 1-methylpiperidin-4-yl, or 4-methylpiperazin-1-yl; and R¹, R², R³, R⁴, R⁶, R^{5a}, R^{5b}, R^{7b}, R^{7c}, R^{7d}, R^{7e}, X, Y, and Z are each as defined herein.

In one embodiment of a compound of Formula (XVIb),

R¹ is hydrogen or OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently hydrogen or C₁₋₆ alkyl, optionally substituted with one, two, three, four, or five substituents Q;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIb),

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently hydrogen or C₁₋₆ alkyl;

R^{7a} is C₆₋₁₄ aryl, heteroaryl, or heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIb),

R¹ is hydrogen or methoxy;

R² is hydrogen;

R³ and R⁴ are hydrogen;

R⁶ is difluoromethyl;

R^{5a} and R^{5b} are each independently hydrogen or C₋₆ alkyl;
R^{7a} is C₆₋₁₄ aryl, monocyclic heteroaryl, or monocyclic heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIb),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, 5- or 6-membered heteroaryl, or 5- or 6-membered heterocyclyl, each of which is optionally substituted with one, two, three, or four substituents Q^{a}; and

R^{7b}, R^{7c}, R^{7d}, and R^{7e} are hydrogen.

In one embodiment of a compound of Formula (XVIb),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a}; and

R^{7b}, R^{7c}, R^{7d}, and R^{7e} are hydrogen.

In one embodiment of a compound of Formula (XVIb),

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, piperidinyl, or piperazinyl, each of which is optionally substituted with one, two, three, or four substituents Q^{a}; and

R^{7b}, R^{7c}, R^{7d}, and R^{7e} are hydrogen.

In one embodiment of a compound of Formula (XVI), (XVIa), or (XVIb), R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}; and R¹, R², R³, R⁴, R⁶, R^{7a}, R^{7b}, R^{7c}, R^{7d}, R^{7e}, R^{1a}, R^{1b}, R^{1c}, and R^{1d} are defined herein elsewhere.

In one embodiment of any of the formulae provided herein,

R¹ is hydrogen or —OR^{1a}, where R^{1a} is C₁₋₆ alkyl, optionally substituted with one, two, three, four, or five substituents Q;

R² is hydrogen;

R³ and R⁴ are hydrogen;

R⁶ is C₁₋₆ alkyl, optionally substituted with one, two, three, four, or five substituents Q;

R^{5a} and R^{5b} are each independently hydrogen or C₁₋₆ alkyl optionally substituted with one, two, three, four, or five substituents Q;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CR^{x}, with the proviso that at least two of X, Y, and Z are N; where R^{x} is a hydrogen or C₁₋₆ alkyl, optionally substituted with one, two, three, or four substituents Q^{a}.

In one embodiment of any of the formulae provided herein,

R¹ is hydrogen or methoxy;

R² is hydrogen;

R³ and R⁴ are hydrogen;

R⁶ is C₁₋₆ alkyl, optionally substituted with one or more halo;

R^{5a} and R^{5b} are each independently hydrogen or C₁₋₆ alkyl;

R^{7a} is C₆₋₁₄ aryl, heteroaryl, or heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein,

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is C₆₋₁₄ aryl, monocyclic heteroaryl, or monocyclic heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein,

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, 5- or 6-membered heteroaryl, or 5- or 6-membered heterocyclyl, each of which is optionally substituted with one, two, three, or four substituents Q^{a};

R^{7b}, R^{7c}, R^{7d}, and R^{7e} are hydrogen; and

X, Y, and Z are each independently N or CH.

In one embodiment of any of the formulae provided herein,

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, pyrimidinyl, pyrrolidinyl, piperidinyl, or piperazinyl, each of which is optionally substituted with one, two, three, or four substituents Q^{a};

R^{7b}, R^{7g}, R^{7d}, and R⁷ are hydrogen; and

X, Y, and Z are each independently N or CH.

In one embodiment of any of the formulae provided herein,

R¹ is hydrogen or methoxy;

R² is hydrogen;

R³ and R⁴ are hydrogen;

R⁶ is difluoromethyl;

R^{5a} and R^{5b} are each independently hydrogen or C₁₋₆ alkyl;

R^{7a} is phenyl, imidazolyl, pyrozolyl, pyridinyl, piperidinyl, or piperazinyl, each of which is optionally substituted with one, two, three, or four substituents Q^{a};

R^{7b}, R^{7g}, R^{7d}, and R^{7e} are hydrogen; and

X, Y, and Z are each independently N or CH.

The groups or variables, R¹, R², R³, R⁴, R⁶, R^{5a}, R^{5b}, R^{5g}, R^{5d}, R^{5g}, R^{5f}, R^{5g}, R^{7a}, R^{7b}, R^{7g}, R^{7d}, R^{7e}, m, n, X, Y, and Z in Formulae provided herein, e.g., Formulae (I), (IX), (X), (XI), (XVI), (Ia), (IXa), (Xa), (XIa), (XVIa), (Ib), (IXb), (Xb), (XIb), (XVIb) are further defined in the embodiments described herein. All combinations of the embodiments provided herein for such groups and/or variables are within the scope of this disclosure.

In certain embodiments, R¹ is hydrogen. In certain embodiments, R¹ is cyano. In certain embodiments, R¹ is halo. In certain embodiments, R¹ is fluoro, chloro, bromo, or iodo. In certain embodiments, R¹ is nitro. In certain embodiments, R¹ is C₁₋₆ alkyl, optionally substituted with one, two, three, four, or five substituents Q as described herein. In certain embodiments, R¹ is C₂₋₆ alkenyl, optionally substituted with one, two, three, four, or five substituents Q as described herein. In certain embodiments, R¹ is C₂₋₆ alkynyl, optionally substituted with one, two, three, four, or five substituents Q as described herein. In certain embodiments, R¹ is C₃₋₁₀ cycloalkyl, optionally substituted with one, two, three, four, or five substituents Q as described herein. In certain embodiments, R¹ is C₆₋₁₄ aryl, optionally substituted with one, two, three, four, or five substituents Q as described herein. In certain embodiments, R¹ is C₇₋₁₅ aralkyl, optionally substituted with one, two, three, four, or five substituents Q as described herein. In certain embodiments, R¹ is heteroaryl, optionally substituted with one, two, three, four, or five substituents Q as described herein. In certain embodiments, R¹ is heterocyclyl, optionally substituted with one, two, three, four, or five substituents Q as described herein.

In certain embodiments, R¹ is C(O)R^{1a}, wherein R^{1a} is as defined herein. In certain embodiments, R¹ is C(O)OR^{1d}, wherein R^{1a} is as defined herein. In certain embodiments, R¹ is C(O)NR^{1b}R^{1c}, wherein R^{1b} and R^{1c} are each as defined herein. In certain embodiments, R¹ is C(NR^{1a})NR^{1b}R^{1c}, wherein R^{1a}, R^{1b}, and R^{1c} are each as defined herein. In certain embodiments, R¹ is —OR^{1a}, wherein R^{1a} is as defined herein. In certain embodiments, R¹ is —OC₁₋₆ alkyl, wherein the alkyl is optionally substituted with one, two, three, four, or five substituents Q as described herein. In certain embodiments, R¹ is methoxy, ethoxy, propoxy, isopropoxy, or 3-dimethylaminopropoxy. In certain embodiments, R¹ is —OC(O)R^{1a}, wherein R^{1a} is as defined herein. In certain embodiments, R¹ is —OC(O)OR^{1d}, wherein R^{1a} is as defined herein. In certain embodiments, R¹ is —OC(O)NR^{1b}R^{1c}, wherein R^{1b} and R^{1c} are each as defined herein. In certain embodiments, R¹ is —OC(═NR^{1a})NR^{1b}R^{1c}, wherein R^{1a}, R^{1b}, and R^{1c} are each as defined herein. In certain embodiments, R¹ is OS(O)R^{1d}, wherein R^{1a} is as defined herein. In certain embodiments, R¹ is —OS(O)₂R^{1a}, wherein R^{1a} is as defined herein. In certain embodiments, R¹ is OS(O)NR^{1b}R^{1c}, wherein R^{1b} and R^{1c} are each as defined herein. In certain embodiments, R¹ is OS(O)₂NR^{1b}R^{1c}, wherein R^{1b} and R^{1c} are each as defined herein. In certain embodiments, R¹ is —NR^{1b}R^{1c}, wherein R^{1b} and R^{1c} are each as defined herein. In certain embodiments, R¹ is —NR^{1d}C(O)R^{1d}, wherein R^{1a} and R^{1d} are each as defined herein. In certain embodiments, R¹ is —NR^{1d}C(O)OR^{1d}, wherein R^{1a} and R^{1d} are each as defined herein. In certain embodiments, R¹ is —NR^{1a}C(O)NR^{1b}R^{1c}, wherein R^{1a}, R^{1b}, and R^{1c} are each as defined herein. In certain embodiments, R¹ is —NR^{1a}C(═NR^{1d})NR^{1b}R^{1c}, wherein R^{1a}, R^{1b}, R^{1c}, and R^{d} are each as defined herein. In certain embodiments, R¹ is —NR^{1a}S(O)R^{1d}, wherein R^{1a} and R^{1d} are each as defined herein. In certain embodiments, R¹ is —NR^{1a}S(O)₂R^{1d}, wherein R^{1a} and R^{1d} are each as defined herein. In certain embodiments, R¹ is —NR^{1a}S(O)NR^{1b}R^{1c}, wherein R^{1a}, R^{1b}, and R^{1c} are each as defined herein. In certain embodiments, R¹ is —NR^{1a}S(O)₂NR^{1b}R^{1c}, wherein R^{1a}, R^{1b}, and R^{1c} are each as defined herein. In certain embodiments, R¹ is —SR^{1d}, wherein R^{1a} is as defined herein. In certain embodiments, R¹ is S(O)R^{1d}, wherein R^{1a} is as defined herein. In certain embodiments, R¹ is S(O)₂R^{1d}, wherein R^{1a} is as defined herein. In certain embodiments, R¹ is S(O)NR^{1b}R^{1c}, wherein R^{1b} and R^{1c} are each as defined herein. In certain embodiments, R¹ is S(O)₂NR^{1b}R^{1c}; wherein R^{1b} and R^{1c} are each as defined herein.

In certain embodiments, R² is hydrogen. In certain embodiments, R² is cyano. In certain embodiments, R² is halo. In certain embodiments, R² is fluoro, chloro, bromo, or iodo. In certain embodiments, R² is nitro. In certain embodiments, R² is C₁₋₆ alkyl, optionally substituted with one, two, three, four, or five substituents Q as described herein. In certain embodiments, R² is C₂₋₆ alkenyl, optionally substituted with one, two, three, four, or five substituents Q as described herein. In certain embodiments, R² is C₂₋₆ alkynyl, optionally substituted with one, two, three, four, or five substituents Q as described herein. In certain embodiments, R² is C₃₋₁₀ cycloalkyl, optionally substituted with one, two, three, four, or five substituents Q as described herein. In certain embodiments, R² is C₃₋₇ cycloalkyl, optionally substituted with one, two, three, four, or five substituents Q as described herein. In certain embodiments, R² is C₆₋₁₄ aryl, optionally substituted with one, two, three, four, or five substituents Q as described herein. In certain embodiments, R² is C₇₋₁₅ aralkyl, optionally substituted with one, two, three, four, or five substituents Q as described herein. In certain embodiments, R² is heteroaryl, optionally substituted with one, two, three, four, or five substituents Q as described herein. In certain embodiments, R² is heterocyclyl, optionally substituted with one, two, three, four, or five substituents Q as described herein.

In certain embodiments, R² is C(O)R^{1a}, wherein R^{1a} is as defined herein. In certain embodiments, R² is C(O)OR^{1a}, wherein R^{1a} is as defined herein. In certain embodiments, R² is C(O)NR^{1b}R^{1c}, wherein R^{1b} and R^{1c} are each as defined herein. In certain embodiments, R² is C(NR^{1a})NR^{1b}R^{1c}, wherein R^{1a}, R^{1b}, and R^{1c} are each as defined herein. In certain embodiments, R² is OR^{1a}, wherein R^{1a} is as defined herein. In certain embodiments, R¹ is —OC₁₋₆ alkyl, wherein the alkyl is optionally substituted with one, two, three, four, or five substituents Q as described herein. In certain embodiments, R¹ is methoxy, ethoxy, propoxy, isopropoxy, or 3-dimethylaminopropoxy. In certain embodiments, R² is —OC(O)R^{1a}, wherein R^{1a} is as defined herein. In certain embodiments, R² is —OC(O)OR^{1a}, wherein R^{1a} is as defined herein. In certain embodiments, R² is —OC(O)NR^{1b}R^{1c}, wherein R^{1b} and R^{1c} are each as defined herein. In certain embodiments, R² is —OC(═NR^{1a})NR^{1b}R^{1c}, wherein R^{1a}, R^{1b} and R^{1c} are each as defined herein. In certain embodiments, R² is OS(O)R^{1a}, wherein R^{1a} is as defined herein. In certain embodiments, R² is OS(O)₂R^{1a}, wherein R^{1a} as defined herein. In certain embodiments, R² is OS(O)NR^{1b}R^{1c}, wherein R^{1b} and R^{1c} are each as defined herein. In certain embodiments, R² is OS(O)₂NR^{1b}R^{1c}, wherein R^{1b} and R^{1c} are each as defined herein. In certain embodiments, R² is —NR^{1b}R^{1c}, wherein R^{1b} and R^{1c} are each as defined herein. In certain embodiments, R² is amino (NH₂). In certain embodiments, R² is —NR^{1a}C(O)R^{1d}, wherein R^{1a} and R^{1d} are each as defined herein. In certain embodiments, R² is —NR^{1a}C(O)OR^{1d}, wherein R^{1a} and R^{1d} are each as defined herein. In certain embodiments, R² is —NR^{1a}C(O)NR^{1b}R^{1c}, wherein R^{1a}, R^{1b}, and R^{1c} are each as defined herein. In certain embodiments, R² is —NR^{1a}C(═NR^{1d})NR^{1b}R^{1c}, wherein R^{1a}, R^{1b}, R^{1c}, and R^{1d} are each as defined herein. In certain embodiments,

R² is —NR^{1a}S(O)R^{1d}, wherein R^{1a} and R^{1d} are each as defined herein. In certain embodiments, R² is —NR^{1a}S(O)₂R^{1d}, wherein R^{1a} and R^{1d} are each as defined herein. In certain embodiments, R² is —NR^{1a}S(O)NR^{1b}R^{1c}, wherein R^{1a}, R^{1b}, and R^{1c} are each as defined herein. In certain embodiments, R² is —NR^{1a}S(O)₂NR^{1b}R^{1c}, wherein R^{1a}, R^{1b} and R^{1c} are each as defined herein. In certain embodiments, R² is —SR^{1a}, wherein R^{1a} is as defined herein. In certain embodiments, R² is S(O)R^{1a}, wherein R^{1a} is as defined herein. In certain embodiments, R² is S(O)₂R^{1a}, wherein R^{1a} is as defined herein. In certain embodiments, R² is S(O)NR^{1b}R^{1c}, wherein R^{1b} and R^{1c} are each as defined herein. In certain embodiments, R² is —S(O)₂NR^{1b}R^{1c}; wherein R^{1b} and R^{1c} are each as defined herein.

In certain embodiments, R³ is hydrogen. In certain embodiments, R³ is C₁₋₆ alkyl, optionally substituted with one, two, three, four, or five substituents Q as described herein. In certain embodiments, R³ is hydrogen, methyl, ethyl, or propyl (e.g., n-propyl, isopropyl, or 2-isopropyl).

In certain embodiments, R⁴ is hydrogen. In certain embodiments, R⁴ is C₁₋₆ alkyl, optionally substituted with one, two, three, four, or five substituents Q as described herein. In certain embodiments, R⁴ is hydrogen, methyl, ethyl, or propyl (e.g., n-propyl, isopropyl, or 2-isopropyl).

In certain embodiments, R³ and R⁴ are linked together to form a bond. In certain embodiments, R³ and R⁴ are linked together to form C₁₋₆ alkylene, optionally substituted with one, two, three, four, or five substituents Q as described herein. In certain embodiments, R³ and R⁴ are linked together to form methylene, ethylene, or propylene, each optionally substituted with one, two, three, four, or five substituents Q as described herein. In certain embodiments, R³ and R⁴ are linked together to form C₁₋₆ heteroalkylene, optionally substituted with one, two, three, four, or five substituents Q as described herein. In certain embodiments, R³ and R⁴ are linked together to form C₂₋₆ alkenylene, optionally substituted with one, two, three, four, or five substituents Q as described herein. In certain embodiments, R³ and R⁴ are linked together to form C₂₋₆ heteroalkenylene, optionally substituted with one, two, three, four, or five substituents Q as described herein.

In certain embodiments, R⁶ is hydrogen. In certain embodiments, R⁶ is C₁₋₆ alkyl, optionally substituted with one, two, three, four, or five substituents Q as described herein. In certain embodiments, R⁶ is C₁₋₆ alkyl, optionally substituted with one or more, in one embodiment, one, two, or three, halo. In certain embodiments, R⁶ is C₁₋₆ alkyl, optionally substituted with one or more, in one embodiment, one, two, or three, fluoro. In certain embodiments, R⁶ is methyl, fluoromethyl, difluoromethyl, or trifluoromethyl. In certain embodiments, R⁶ is difluoromethyl. In certain embodiments, R⁶ is —S—C₁₋₆ alkyl, wherein the alkyl is optionally substituted with one, two, three, four, or five substituents Q as described herein. In certain embodiments, R⁶ is S(O)C₁₋₆ alkyl, wherein the alkyl is optionally substituted with one, two, three, four, or five substituents Q as described herein. In certain embodiments, R⁶ is SO₂C₁₋₆ alkyl, wherein the alkyl is optionally substituted with one, two, three, four, or five substituents Q as described herein.

In certain embodiments, R^{5a} is hydrogen. In certain embodiments, R^{5a} is not hydrogen. In certain embodiments, R^{5a} is halo. In certain embodiments, R^{5a} is fluoro, chloro, bromo, or iodo. In certain embodiments, R^{5a} is C₁₋₆ alkyl, optionally substituted with one, two, three, four, or five substituents Q as described herein. In certain embodiments, R^{5a} is methyl, ethyl, propyl, or butyl, each optionally substituted with one, two, three, four, or five substituents Q as described herein. In certain embodiments, R^{5a} is methyl, ethyl, n-propyl, isopropyl, n-butyl, isobutyl, or t-butyl. In certain embodiments, R^{5a} is methyl. In certain embodiments, R^{5a} is C₂₋₆ alkenyl, optionally substituted with one, two, three, four, or five substituents Q as described herein. In certain embodiments, R^{5a} is C₂₋₆ alkynyl, optionally substituted with one, two, three, four, or five substituents Q as described herein. In certain embodiments, R^{5a} is C₃₋₁₀ cycloalkyl, optionally substituted with one, two, three, four, or five substituents Q as described herein. In certain embodiments, R^{5a} is C₃₋₇ cycloalkyl, optionally substituted with one, two, three, four, or five substituents Q as described herein. In certain embodiments, R^{5a} is C₆₋₁₄ aryl, optionally substituted with one, two, three, four, or five substituents Q as described herein. In certain embodiments, R^{5a} is C₇₋₁₅ aralkyl, optionally substituted with one, two, three, four, or five substituents Q as described herein. In certain embodiments, R^{5a} is heteroaryl, optionally substituted with one, two, three, four, or five substituents Q as described herein. In certain embodiments, R^{5a} is heterocyclyl, optionally substituted with one, two, three, four, or five substituents Q as described herein.

In certain embodiments, R^{5a} is —C(O)R^{1a}, wherein R^{1a} is as defined herein. In certain embodiments, R^{5a} is —C(O)OR^{1a}, wherein R^{1a} is as defined herein. In certain embodiments, R^{5a} is —C(O)OR^{1a}, wherein R^{1a} is C₁₋₆ alkyl, optionally substituted with one, two, three, four, or five substituents Q as described herein. In certain embodiments, R^{5a} is —C(O)OCH₃. In certain embodiments, R^{5a} is —C(O)NR^{1b}R^{1c}, wherein R^{1b} and R^{1c} are each as defined herein. In certain embodiments, R^{5a} is —C(NR^{1a})NR^{1b}R^{1c}, wherein R^{1a}, R^{1b}, and R^{1c} are each as defined herein. In certain embodiments, R^{5a} is —OR^{1a}, wherein R^{1a} is as defined herein. In certain embodiments, R^{5a} is —OC(O)R^{1a}, wherein R^{1a} is as defined herein. In certain embodiments, R^{5a} is —OC(O)OR^{1a}, wherein R^{1a} is as defined herein. In certain embodiments, R^{5a} is —OC(O)NR^{1b}R^{1c}, wherein R^{1b} and R^{1c} are each as defined herein. In certain embodiments, R^{5a} is —OC(═NR^{1a})NR^{1b}R^{1c}, wherein R^{1a}, R^{1b}, and R^{1c} are each as defined herein. In certain embodiments, R^{5a} is —OS(O)R^{1a}, wherein R^{1a} is as defined herein. In certain embodiments, R^{5a} is —OS(O)₂R^{1a}, wherein R^{1a} is as defined herein. In certain embodiments, R^{5a} is —OS(O)NR^{1b}R^{1c}, wherein R^{1b} and R^{1c} are each as defined herein. In certain embodiments, R^{5a} is —OS(O)₂NR^{1b}R^{1c}, wherein R^{1b} and R^{1c} are each as defined herein. In certain embodiments, R^{5a} is —NR^{1b}R^{1c}, wherein R^{1b} and R^{1c} are each as defined herein. In certain embodiments, R^{5a} is amino (—NH₂). In certain embodiments, R^{5a} is —NR^{1a}C(O)R^{1d}, wherein R^{1a} and R^{1d} are each as defined herein. In certain embodiments, R^{5a} is —NR^{1a}C(O)OR^{1d}, wherein R^{1a} and R^{1d} are each as defined herein. In certain embodiments, R^{5a} is —NR^{1a}C(O)NR^{1d}, wherein R^{1a}, R^{1b}, and R^{1c} are each as defined herein. In certain embodiments, R^{5a} is —NR^{1a}C(═NR^{1d})NR^{1b}R^{1c}, wherein R^{1a}, R^{1b}, R^{1c}, and R^{1d} are each as defined herein. In certain embodiments, R^{5a} is —NR^{1a}S(O)R^{1d}, wherein R^{1a} and R^{1d} are each as defined herein. In certain embodiments, R^{5a} is —NR^{1a}S(O)₂R^{1d}, wherein R^{1a} and R^{1d} are each as defined herein. In certain embodiments, R^{5a} is —NR^{1a}S(O)NR^{1b}R^{1c}, wherein R^{1a}, R^{1b}, and R^{1c} are each as defined herein. In certain embodiments, R^{5a} is —NR^{1a}S(O)₂NR^{1b}R^{1c}, wherein R^{1a}, R^{1b} and R^{1c} are each as defined herein. In certain embodiments, R^{5a} is —SR^{1a}, wherein R^{1a} is as defined herein. In certain embodiments, R^{5a} is —S(O)R^{1a}, wherein R^{1a} is as defined herein. In certain embodiments, R^{5a} is —S(O)₂R^{1a}, wherein R^{1a} is as defined herein. In certain embodiments, R^{5a} is —S(O)NR^{1b}R^{1c}, wherein R^{1b} and R^{1c} are each as defined herein. In certain embodiments, R^{5a} is —S(O)₂NR^{1b}R^{1c}; wherein R^{1b} and R^{1c} are each as defined herein.

In certain embodiments, R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl, each of which is optionally substituted with one, two, three, four, or five substituents Q; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}. In certain embodiments, R^{5a} is (a) hydrogen or halo; or (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl, each of which is optionally substituted with one, two, three, four, or five substituents Q.

In certain embodiments, R^{5b} is halo. In certain embodiments, R^{5b} is fluoro, chloro, bromo, or iodo. In certain embodiments, R^{5b} is C₁₋₆ alkyl, optionally substituted with one, two, three, four, or five substituents Q as described herein. In certain embodiments, R^{5b} is methyl, ethyl, propyl, or butyl, each optionally substituted with one, two, three, four, or five substituents Q as described herein. In certain embodiments, R^{5b} is methyl, ethyl, n-propyl, isopropyl, n-butyl, isobutyl, or t-butyl. In certain embodiments, R^{5b} is methyl. In certain embodiments, R^{5b} is C₂₋₆ alkenyl, optionally substituted with one, two, three, four, or five substituents Q as described herein. In certain embodiments, R^{5b} is C₂₋₆ alkynyl, optionally substituted with one, two, three, four, or five substituents Q as described herein. In certain embodiments, R^{5b} is C₃₋₁₀ cycloalkyl, optionally substituted with one, two, three, four, or five substituents Q as described herein. In certain embodiments, R^{5b} is C₃₋₇ cycloalkyl, optionally substituted with one, two, three, four, or five substituents Q as described herein. In certain embodiments, R^{5b} is C₆₋₁₄ aryl, optionally substituted with one, two, three, four, or five substituents Q as described herein. In certain embodiments, R^{5b} is C₇₋₁₅ aralkyl, optionally substituted with one, two, three, four, or five substituents Q as described herein. In certain embodiments, R^{5b} is heteroaryl, optionally substituted with one, two, three, four, or five substituents Q as described herein. In certain embodiments, R^{5b} is heterocyclyl, optionally substituted with one, two, three, four, or five substituents Q as described herein. In certain embodiments, R^{5b} is not heterocyclyl.

In certain embodiments, R^{5b} is C(O)R^{1a}, wherein R^{1a} is as defined herein. In certain embodiments, R^{5b} is C(O)OR^{1a}, wherein R^{1a} is as defined herein. In certain embodiments, R^{5b} is —C(O)OR^{1d}, wherein R^{1a} is C₁₋₆ alkyl, optionally substituted with one, two, three, four, or five substituents Q as described herein. In certain embodiments, R^{5b} is C(O)OCH₃. In certain embodiments, R^{5b} is —C(O)NR^{1b}R^{1c}, wherein R^{1b} and R^{1c} are each as defined herein. In certain embodiments, R^{5b} is —C(NR^{1a})NR^{1b}R^{1c}, wherein R^{1a}, R^{1b}, and R^{1c} are each as defined herein. In certain embodiments, R^{5b} is —OR^{1a}, wherein R^{1a} is as defined herein. In certain embodiments, R^{5b} is —OC(O)R^{1d}, wherein R^{1a} is as defined herein. In certain embodiments, R^{5b} is —OC(O)OR^{1d}, wherein R^{1a} is as defined herein. In certain embodiments, R^{5b} is —OC(O)NR^{1b}R^{1c}, wherein R^{1b} and R^{1c} are each as defined herein. In certain embodiments, R^{5b} is —OC(═NR^{1a})NR^{1b}R^{1c}, wherein R^{1a}, R^{1b}, and R^{1c} are each as defined herein. In certain embodiments, R^{5b} is OS(O)R^{1d}, wherein R^{1a} is as defined herein. In certain embodiments, R^{5b} is —OS(O)₂R^{1d}, wherein R^{1a} is as defined herein. In certain embodiments, R^{5b} is OS(O)NR^{1b}R^{1c}, wherein R^{1b} and R^{1c} are each as defined herein. In certain embodiments, R^{5b} is OS(O)₂NR^{1b}R^{1c}, wherein R^{1b} and R^{1c} are each as defined herein. In certain embodiments, R^{5b} is —NR^{1b}R^{1c}, wherein R^{1b} and R^{1c} are each as defined herein. In certain embodiments, R^{5b} is amino (NH₂). In certain embodiments, R^{5b} is —NR^{1d}C(O)R^{1d}, wherein R^{1a} and R^{1d} are each as defined herein. In certain embodiments, R^{5b} is —NR^{1d}C(O)OR^{1d}, wherein R^{1a} and R^{1d} are each as defined herein. In certain embodiments, R^{5b} is —NR^{1a}C(O)NR^{1b}R^{1c}, wherein R^{1a}, R^{1b}, and R^{1c} are each as defined herein. In certain embodiments, R^{5b} is —NR^{1a}C(═NR^{1d})NR^{1b}R^{1c}, wherein R^{1a}, R^{1b}, R^{1c} and R^{1d} are each as defined herein. In certain embodiments,

R^{5b} is —NR^{1a}S(O)R^{1d}, wherein R^{1a} and R^{1d} are each as defined herein. In certain embodiments, R^{5b} is —NR^{1a}S(O)₂R^{1d}, wherein R^{1a} and R^{1d} are each as defined herein. In certain embodiments, R^{5b} is —NR^{1a}S(O)NR^{1b}R^{1c}, wherein R^{1a}, R^{1b}, and R^{1c} are each as defined herein. In certain embodiments, R^{5b} is —NR^{1a}S(O)₂NR^{1b}R^{1c}, wherein R^{1a}, R^{1b}, and R^{1c} are each as defined herein. In certain embodiments, R^{5b} is —SR^{1a}, wherein R^{1a} is as defined herein. In certain embodiments, R^{5b} is S(O)R^{1a}, wherein R^{1a} is as defined herein. In certain embodiments, R^{5b} is S(O)₂R^{1a}, wherein R^{1a} is as defined herein. In certain embodiments, R^{5b} is S(O)NR^{1b}R^{1c}, wherein R^{1b} and R^{1c} are each as defined herein. In certain embodiments, R^{5b} is S(O)₂NR^{1b}R^{1c}; wherein R^{1b} and R^{1c} are each as defined herein.

In certain embodiments, R^{5a} and R^{5b} are each independently methyl, ethyl, n-propyl, isopropyl, n-butyl, isobutyl, or t-butyl, each optionally substituted with one, two, three, four, or five substituents Q as described herein. In certain embodiments, R^{5a} and R^{5b} are each independently methyl, ethyl, n-propyl, isopropyl, n-butyl, isobutyl, or t-butyl, each optionally substituted with one or more halo. In certain embodiments, R^{5a} and R^{5b} are each independently methyl, ethyl, n-propyl, isopropyl, n-butyl, isobutyl, or t-butyl. In certain embodiments, R^{5a} and R^{5b} are each methyl.

In certain embodiments, R^{5c} is C₆₋₁₄ aryl, optionally substituted with one, two, three, four, or five substituents Q as described herein. In certain embodiments, R^{5b} is C₆₋₁₄ aryl substituted at the 2-position with one substituent Q as described herein. In certain embodiments, R^{5c} is phenyl or naphthyl, each optionally substituted with one, two, three, four, or five substituents Q as described herein. In certain embodiments, R^{5c} is phenyl, naphtha-1-yl, or naphtha-2-yl, each optionally substituted with one, two, three, four, or five substituents Q as described herein. In certain embodiments, R^{5c} is phenyl, 4-chlorophenyl, 4-methoxyphenyl, or naphtha-2-yl. In certain embodiments, R^{5c} is heteroaryl, optionally substituted with one, two, three, four, or five substituents Q as described herein. In certain embodiments, R^{5c} is monocyclic heteroaryl, optionally substituted with one, two, three, four, or five substituents Q as described herein. In certain embodiments, R^{5c} is 5- or 6-membered heteroaryl, optionally substituted with one, two, three, four, or five substituents Q as described herein. In certain embodiments, R^{5c} is bicyclic heteroaryl, optionally substituted with one, two, three, four, or five substituents Q as described herein.

In certain embodiments, R^{5c} is (CR^{5f}R^{5g})ₙ—(C₆₋₁₄ aryl), wherein the C₆₋₁₄ aryl is optionally substituted with one, two, three, four, or five substituents Q as described herein. In certain embodiments, R^{5c} is benzyl, 2-phenethyl, 3-phenylpropyl, or 4-phenylbutyl, wherein each of the phenyl moiety is optionally substituted with one, two, three, four, or five substituents Q as described herein. In certain embodiments, R^{5c} is benzyl, 2-phenethyl, 3-phenylpropyl, or 4-phenylbutyl. In certain embodiments, R^{5c} is benzyl, fluorobenzyl, chlorobenzyl, bromobenzyl, cyanobenzyl, methylbenzyl, or methoxybenzyl. In certain embodiments, R^{5c} is (naphthalen-1-yl)methyl, (naphthalen-2-yl)methyl 2-(naphthalen-1-yl)ethyl, 2-(naphthalen-2-yl)ethyl, 3-(naphthalen-1-yl)propyl, 3-(naphthalen-2-yl)propyl, 4-(naphthalen-1-yl)butyl, or 4-(naphthalen-2-yl)butyl, wherein each of the naphthyl moiety is optionally substituted with one, two, three, four, or five substituents Q as described herein. In certain embodiments, n is 0 or 1. In one embodiment, n is 1. In one embodiment, n is 1, 2, 3, or 4. In certain embodiments, R^{5C} is —CH₂—(C₆₋₁₄ aryl), wherein the C₆₋₁₄ aryl is optionally substituted with one, two, three, four, or five substituents Q as described herein. In certain embodiments, R^{5c} is —C(CH₃)₂—(C₆₋₁₄ aryl), wherein the C₆₋₁₄ aryl is optionally substituted with one, two, three, four, or five substituents Q as described herein. In certain embodiments, R^{5c} is —CH₂-phenyl or —CH₂-naphthyl, wherein the phenyl or naphthyl is each optionally substituted with one, two, three, four, or five substituents Q as described herein, such as, e.g., optionally substituted with one or more F, Cl, Br, I, —CN, —CH₃, —CF₃, —OCH₃, or —OCF₃. In certain embodiments, R^{5c} is —CH₂-phenyl, —CH₂-naphtha-1-yl, or —CH₂-naphtha-2-yl, wherein the phenyl or naphthyl is each optionally substituted with one, two, three, four, or five substituents Q as described herein, such as, e.g., optionally substituted with one or more F, Cl, Br, I, —CN, —CH₃, —CF₃, —OCH₃, or —OCF₃. In certain embodiments, R^{5c} is —CH₂-phenyl, —CH₂-naphtha-1-yl, or —CH₂-naphtha-2-yl, wherein the phenyl or naphthyl is each optionally substituted with one or more F, Cl, Br, I, —CN, —CH₃, —CF₃, —OCH₃, —OCF₃. In other embodiments, R^{5c} is —CH₂-phenyl, —CH₂-naphtha-1-yl, or —CH₂-naphtha-2-yl, wherein the phenyl or naphthyl is each optionally substituted with one or more F, Cl, Br, I, —CN, —CH₃, —CF₃, —OCH₃, —OCF₃, —O—(C₁₋₄ alkylene)-N—(C₁₋₄ alkyl)₂ (e.g., —O—CH₂CH₂—N(CH₃)₂), —O-heterocyclyl (e.g., —O—(N-methylpiperidinyl) or —O-piperidinyl), —O-heteroaryl (e.g., —O-pyridyl), —NH-heterocyclyl (e.g., —NH—(N-methylpiperidinyl), —NH—(N-methylpyrrolidinyl), —NH-piperidinyl, or —NH-pyrrolidinyl), —NH-heteroaryl (e.g., —NH-pyridyl), —NCH₃-heterocyclyl (e.g., —NCH₃-(N-methylpiperidinyl), —NCH₃-(N-methylpyrrolidinyl), —NCH₃-piperidinyl, or —NCH₃-pyrrolidinyl), —NCH₃-heteroaryl (e.g., —NCH₃— pyridyl), heterocyclyl (e.g., piperidinyl, piperazinyl, N-methylpiperidinyl, or N-methylpiperazinyl), or heteroaryl (e.g., pyridyl or imidazolyl). In certain embodiments, R^{5c} is —CH₂-phenyl, —C(CH₃)₂-phenyl, —CH₂-(2-methylphenyl), —CH₂-(2-methoxylphenyl), —CH₂-(2-fluorophenyl), —CH₂-(2-chlorophenyl), —CH₂-(2-bromophenyl), —CH₂-(3-methylphenyl), —CH₂-(3-methoxylphenyl), —CH₂-(3-fluorophenyl), —CH₂-(3-chlorophenyl), —CH₂-(3-bromophenyl), —CH₂-(4-methylphenyl), —CH₂-(4-methoxylphenyl), —CH₂-(4-fluorophenyl), —CH₂-(4-chlorophenyl), —CH₂-(4-bromophenyl), —CH₂-naphtha-1-yl, or —CH₂-naphtha-2-yl.

In certain embodiments, R^{5c} is —(CR^{5f}R^{5g})-(C₆₋₁₄ aryl), wherein the C₆₋₁₄ aryl is optionally substituted with one, two, three, four, or five substituents Q as described herein, and wherein R^{5f} and R^{5g} together with the carbon atom to which they are attached form a 3- to 6-membered cycloalkyl or heterocyclyl. In one embodiment, R^{5c} is -cyclopropyl-phenyl. In one embodiment, R⁵ is -cyclobutyl-phenyl. In one embodiment, R^{5c} is -cyclopentyl-phenyl. In one embodiment, R⁵ is -cyclohexyl-phenyl.

In certain embodiments, R^{5c} is —(CR^{5f}R^{5g})ₙ-heteroaryl, wherein the heteroaryl is optionally substituted with one, two, three, four, or five substituents Q as described herein, wherein n is defined herein elsewhere. In certain embodiments, R^{5c} is —CH₂-(monocyclic heteroaryl), wherein the heteroaryl is optionally substituted with one, two, three, four, or five substituents Q as described herein. In certain embodiments, R^{5c} is —CH₂-(5- or 6-membered heteroaryl), wherein the heteroaryl is optionally substituted with one, two, three, four, or five substituents Q as described herein. In certain embodiments, R^{5c} is —CH₂-(bicyclic heteroaryl), wherein the heteroaryl is optionally substituted with one, two, three, four, or five substituents as described herein.

In certain embodiments, R^{5d} is hydrogen. In certain embodiments, R^{5d} is halo. In certain embodiments, R^{5d} is fluoro, chloro, bromo, or iodo. In certain embodiments, R^{5d} is C₁₋₆ alkyl, optionally substituted with one, two, three, four, or five substituents Q as described herein. In certain embodiments, R^{5d} is methyl, optionally substituted with one, two, three, four, or five substituents Q as described herein. In certain embodiments, R^{5d} is methyl. In certain embodiments, R^{5d} is methyl, ethyl, propyl, or butyl, each optionally substituted with one, two, three, four, or five substituents Q as described herein. In certain embodiments, R^{5d} is methyl, ethyl, n-propyl, isopropyl, n-butyl, isobutyl, or t-butyl. In certain embodiments, R^{5d} is C₂₋₆ alkenyl, optionally substituted with one, two, three, four, or five substituents Q as described herein. In certain embodiments, R^{5d} is C₂₋₆ alkynyl, optionally substituted with one, two, three, four, or five substituents Q as described herein. In certain embodiments, R^{5d} is C₃₋₁₀ cycloalkyl, optionally substituted with one, two, three, four, or five substituents Q as described herein. In certain embodiments, R^{5d} is C₆₋₁₄ aryl, optionally substituted with one, two, three, four, or five substituents Q as described herein. In certain embodiments, R^{5d} is C₇₋₁₅ aralkyl, optionally substituted with one, two, three, four, or five substituents Q as described herein. In certain embodiments, R^{5d} is heteroaryl, optionally substituted with one, two, three, four, or five substituents Q as described herein. In certain embodiments, R^{5d} is heterocyclyl, optionally substituted with one, two, three, four, or five substituents Q as described herein.

In certain embodiments, R^{5d} is C(O)R^{1a}, wherein R^{1a} is as defined herein. In certain embodiments, R^{5d} is C(O)OR^{1a}, wherein R^{1a} is as defined herein. In certain embodiments, R^{5d} is —C(O)OR^{1a}, wherein R^{1a} is C₁₋₆ alkyl, optionally substituted with one, two, three, four, or five substituents Q as described herein. In certain embodiments, R^{5d} is C(O)OCH₃. In certain embodiments, R^{5d} is —C(O)NR^{1b}R^{1c}, wherein R^{1b} and R^{1c} are each as defined herein. In certain embodiments, R^{5d} is —C(NR^{1a})NR^{1b}R^{1c}, wherein R^{1a}, R^{1b}, and R^{1c} are each as defined herein. In certain embodiments, R^{5d} is —OR^{1a}, wherein R^{1a} is as defined herein. In certain embodiments, R^{5d} is —OC(O)R^{1a}, wherein R^{1a} is as defined herein. In certain embodiments, R^{5d} is —OC(O)OR^{1a}, wherein R^{1a} is as defined herein. In certain embodiments, R^{5d} is —OC(O)NR^{1b}R^{1c}, wherein R^{1b} and R^{1c} are each as defined herein. In certain embodiments, R^{5d} is —OC(═NR^{1a})NR^{1b}R^{1c}, wherein R^{1a}, R^{1b}, and R^{1c} are each as defined herein. In certain embodiments, R^{5d} is OS(O)R^{1a}, wherein R^{1a} is as defined herein. In certain embodiments, R^{5d} is —OS(O)₂R^{1a}, wherein R^{1a} is as defined herein. In certain embodiments, R^{5d} is OS(O)NR^{1b}R^{1c}, wherein R^{1b} and R^{1c} are each as defined herein. In certain embodiments, R^{5d} is OS(O)₂NR^{1b}R^{1c}, wherein R^{1b} and R^{1c} are each as defined herein. In certain embodiments, R^{5d} is —NR^{1b}R^{1c}, wherein R^{1b} and R^{1c} are each as defined herein. In certain embodiments, R^{5d} is amino (NH₂). In certain embodiments, R^{5d} is —NR^{1a}C(O)R^{1d}, wherein R^{1a} and R^{1d} are each as defined herein. In certain embodiments, R^{5d} is —NR^{1a}C(O)OR^{1d}, wherein R^{1a} and R^{1d} are each as defined herein. In certain embodiments, R^{5d} is —NR^{1a}C(O)NR^{1b}R^{1c}, wherein R^{1a}, R^{1b}, and R^{1c} are each as defined herein. In certain embodiments, R^{5d} is —NR^{1a}C(═NR^{1d})NR^{1b}R^{1c}, wherein R^{1a}, R^{1b}, R^{1c}, and R^{1d} are each as defined herein. In certain embodiments, R^{5d} is —NR^{1a}S(O)R^{1d}, wherein R^{1a} and R^{1d} are each as defined herein. In certain embodiments, R^{5d} is —NR^{1a}S(O)₂R^{1d}, wherein R^{1a} and R^{1d} are each as defined herein. In certain embodiments, R^{5d} is —NR^{1a}S(O)NR^{1b}R^{1c}, wherein R^{1a}, R^{1b}, and R^{1c} are each as defined herein. In certain embodiments, R^{5d} is —NR^{1a}S(O)₂NR^{1b}R^{1c}, wherein R^{1a}, R^{1b}, and R^{1c} are each as defined herein. In certain embodiments, R^{5d} is —SR^{1a}, wherein R^{1a} is as defined herein. In certain embodiments, R^{5d} is S(O)R^{1a}, wherein R^{1a} is as defined herein. In certain embodiments, R^{5d} is S(O)₂R^{1a}, wherein R^{1a} is as defined herein. In certain embodiments, R^{5d} is S(O)NR^{1b}R^{1c}, wherein R^{1b} and R^{1c} are each as defined herein. In certain embodiments, R^{5d} is S(O)₂NR^{1b}R^{1c}; wherein R^{1b} and R^{1c} are each as defined herein.

In certain embodiments, R^{5e} is hydrogen. In certain embodiments, R^{5e} is halo. In certain embodiments, R^{5e} is fluoro, chloro, bromo, or iodo. In certain embodiments, R^{5e} is C₁₋₆ alkyl, optionally substituted with one, two, three, four, or five substituents Q as described herein. In certain embodiments, Rye is methyl, optionally substituted with one, two, three, four, or five substituents Q as described herein. In certain embodiments, R^{5e} is methyl. In certain embodiments, R^{5e} is methyl, ethyl, propyl, or butyl, each optionally substituted with one, two, three, four, or five substituents Q as described herein. In certain embodiments, R^{5e} is methyl, ethyl, n-propyl, isopropyl, n-butyl, isobutyl, or t-butyl. In certain embodiments, R^{5e} is C₂₋₆ alkenyl, optionally substituted with one, two, three, four, or five substituents Q as described herein. In certain embodiments, R^{5e} is C₂₋₆ alkynyl, optionally substituted with one, two, three, four, or five substituents Q as described herein. In certain embodiments, R^{5e} is C₃₋₁₀ cycloalkyl, optionally substituted with one, two, three, four, or five substituents Q as described herein. In certain embodiments, R^{5e} is C₆₋₁₄ aryl, optionally substituted with one, two, three, four, or five substituents Q as described herein. In certain embodiments, R^{5e} is C₇₋₁₅ aralkyl, optionally substituted with one, two, three, four, or five substituents Q as described herein. In certain embodiments, R^{5e} is heteroaryl, optionally substituted with one, two, three, four, or five substituents Q as described herein. In certain embodiments, Rye is heterocyclyl, optionally substituted with one, two, three, four, or five substituents Q as described herein.

In certain embodiments, R^{5e} is C(O)R^{1a}, wherein R^{1a} is as defined herein. In certain embodiments, R^{5e} is C(O)OR^{1a}, wherein R^{1a} is as defined herein. In certain embodiments, R^{5e} is —C(O)OR^{1a}, wherein R^{1a} is C₁₋₆ alkyl, optionally substituted with one, two, three, four, or five substituents Q as described herein. In certain embodiments, R^{5e} is C(O)OCH₃. In certain embodiments, R^{5e} is —C(O)NR^{1b}R^{1c}, wherein R^{1b} and R^{1c} are each as defined herein. In certain embodiments, R^{5e} is —C(NR^{1a})NR^{1b}R^{1c}, wherein R^{1a}, R^{1b}, and R^{1c} are each as defined herein. In certain embodiments, R^{5e} is —OR^{1a}, wherein R^{1a} is as defined herein. In certain embodiments, R^{5e} is —OC(O)R^{1a}, wherein R^{1a} is as defined herein. In certain embodiments, R^{5e} is —OC(O)OR^{1a}, wherein R^{1a} is as defined herein. In certain embodiments, R^{5e} is —OC(O)NR^{1b}R^{1c}, wherein R^{1b} and R^{1c} are each as defined herein. In certain embodiments, R^{5e} is —OC(═NR^{1a})NR^{1b}R^{1c}, wherein R^{1a}, R^{1b}, and R^{1c} are each as defined herein. In certain embodiments, R^{5e} is OS(O)R^{1a}, wherein R^{1a} is as defined herein. In certain embodiments, R^{5e} is —OS(O)₂R^{1a}, wherein R^{1a} is as defined herein. In certain embodiments, R^{5e} is OS(O)NR^{1b}R^{1c}, wherein R^{1b} and R^{1c} are each as defined herein. In certain embodiments, R^{5e} is OS(O)₂NR^{1b}R^{1c}, wherein R^{1b} and R^{1c} are each as defined herein. In certain embodiments, R^{5e} is —NR^{1b}R^{1c}, wherein R^{1b} and R^{1c} are each as defined herein. In certain embodiments, R^{5e} is amino (NH₂). In certain embodiments, R^{5e} is —NR^{1a}C(O)R^{1d}, wherein R^{1a} and R^{1d} are each as defined herein. In certain embodiments, R^{5e} is —NR^{1a}C(O)OR^{1d}, wherein R^{1a} and R^{1d} are each as defined herein. In certain embodiments, R^{5e} is —NR^{1a}C(O)NR^{1b}R^{1c}, wherein R^{1a}, R^{1b}, and R^{1c} are each as defined herein. In certain embodiments, R^{5e} is —NR^{1a}C(═NR^{1d})NR^{1b}R^{1c}, wherein R^{1a}, R^{1b}, R^{1c}, and R^{1d} are each as defined herein. In certain embodiments, R^{5e} is —NR^{1a}S(O)R^{1d}, wherein R^{1a} and R^{1d} are each as defined herein. In certain embodiments, R^{5e} is —NR^{1a}S(O)₂R^{1d}, wherein R^{1a} and R^{1d} are each as defined herein. In certain embodiments, R^{5e} is —NR^{1a}S(O)NR^{1b}R^{1c}, wherein R^{1a}, R^{1b}, and R^{1c} are each as defined herein. In certain embodiments, R^{5e} is —NR^{1a}S(O)₂NR^{1b}R^{1c}, wherein R^{1a}, R^{1b}, and R^{1c} are each as defined herein. In certain embodiments, R^{5e} is —SR^{1a}, wherein R^{1a} is as defined herein. In certain embodiments, R^{5e} is S(O)R^{1a}, wherein R^{1a} is as defined herein. In certain embodiments, R^{5e} is S(O)₂R^{1a}, wherein R^{1a} is as defined herein. In certain embodiments, R^{5e} is S(O)NR^{1b}R^{1c}, wherein R^{1b} and R^{1c} are each as defined herein. In certain embodiments, R^{5e} is S(O)₂NR^{1b}R^{1c}; wherein R^{1b} and R^{1c} are each as defined herein.

In certain embodiments, R^{5f} is hydrogen. In certain embodiments, R^{5f} is halo. In certain embodiments, R^{5f} is fluoro, chloro, bromo, or iodo. In certain embodiments, R^{5f} is C₁₋₆ alkyl, optionally substituted with one, two, three, four, or five substituents Q as described herein. In certain embodiments, R^{5f} is methyl, optionally substituted with one, two, three, four, or five substituents Q as described herein. In certain embodiments, R^{5f} is methyl. In certain embodiments, R^{5f} is methyl, ethyl, propyl, or butyl, each optionally substituted with one, two, three, four, or five substituents Q as described herein. In certain embodiments, R^{5f} is methyl, ethyl, n-propyl, isopropyl, n-butyl, isobutyl, or t-butyl. In certain embodiments, R^{5f} is C₂₋₆ alkenyl, optionally substituted with one, two, three, four, or five substituents Q as described herein. In certain embodiments, R^{5f} is C₂₋₆ alkynyl, optionally substituted with one, two, three, four, or five substituents Q as described herein. In certain embodiments, R^{5f} is C₃₋₁₀ cycloalkyl, optionally substituted with one, two, three, four, or five substituents Q as described herein. In certain embodiments, R^{5f} is C₆₋₁₄ aryl, optionally substituted with one, two, three, four, or five substituents Q as described herein. In certain embodiments, R^{5f} is C₇₋₁₅ aralkyl, optionally substituted with one, two, three, four, or five substituents Q as described herein. In certain embodiments, R^{5f} is heteroaryl, optionally substituted with one, two, three, four, or five substituents Q as described herein. In certain embodiments, R^{5f} is heterocyclyl, optionally substituted with one, two, three, four, or five substituents Q as described herein.

In certain embodiments, R^{5f} is C(O)R^{1a}, wherein R^{1a} is as defined herein. In certain embodiments, R^{5f} is C(O)OR^{1a}, wherein R^{1a} is as defined herein. In certain embodiments, R^{5f} is —C(O)OR^{1a}, wherein R^{1a} is C₁₋₆ alkyl, optionally substituted with one, two, three, four, or five substituents Q as described herein. In certain embodiments, R^{5f} is C(O)OCH₃. In certain embodiments, R^{5f} is —C(O)NR^{1b}R^{1c} wherein R^{1b} and R^{1c} are each as defined herein. In certain embodiments, R^{5f} is —C(NR^{1a})NR^{1b}R^{1c}, wherein R^{1a}, R^{1b}, and R^{1c} are each as defined herein. In certain embodiments, R^{5f} is —OR^{1a}, wherein R^{1a} is as defined herein. In certain embodiments, R^{5f} is —OC(O)R^{1a}, wherein R^{1a} is as defined herein. In certain embodiments, R^{5f} is —OC(O)OR^{1a}, wherein R^{1a} is as defined herein. In certain embodiments, R^{5f} is —OC(O)NR^{1b}R^{1c}, wherein R^{1b} and R^{1c} are each as defined herein. In certain embodiments, R^{5f} is —OC(═NR^{1a})NR^{1b}R^{1c}, wherein R^{1a}, R^{1b}, and R^{1c} are each as defined herein. In certain embodiments, R^{5f} is OS(O)R^{1a}, wherein R^{1a} is as defined herein. In certain embodiments, R^{5f} is —OS(O)₂R^{1a}, wherein R^{1a} is as defined herein. In certain embodiments, R^{5f} is OS(O)NR^{1b}R^{1c}, wherein R^{1b} and R^{1c} are each as defined herein. In certain embodiments, R^{5f} is OS(O)₂NR^{1b}R^{1c}, wherein R^{1b} and R^{1c} are each as defined herein. In certain embodiments, R^{5f} is —NR^{1b}R^{1c}, wherein R^{1b} and R^{1c} are each as defined herein. In certain embodiments, R^{5f} is amino (NH₂). In certain embodiments, R^{5f} is —NR^{1a}C(O)R^{1d}, wherein R^{1a} and R^{1d} are each as defined herein. In certain embodiments, R^{5f} is —NR^{1a}C(O)OR^{1d}, wherein R^{1a} and R^{1d} are each as defined herein. In certain embodiments, R^{5f} is —NR^{1a}C(O)NR^{1b}R^{1c}, wherein R^{1a}, R^{1b}, and R^{1c} are each as defined herein. In certain embodiments, R^{5f} is —NR^{1a}C(═NR^{1d})NR^{1b}R^{1c}, wherein R^{1a}, R^{1b}, R^{1c}, and R^{1d} are each as defined herein. In certain embodiments, R^{5f} is —NR^{1a}S(O)R^{1d}, wherein R^{1a} and R^{1d} are each as defined herein. In certain embodiments, R^{5f} is —NR^{1a}S(O)₂R^{1d}, wherein R^{1a} and R^{1d} are each as defined herein. In certain embodiments, R^{5f} is —NR^{1a}S(O)NR^{1b}R^{1c}, wherein R^{1a}, R^{1b}, and R^{1c} are each as defined herein. In certain embodiments, R^{5f} is —NR^{1a}S(O)₂NR^{1b}R^{1c}, wherein R^{1a}, R^{1b} and R^{1c} are each as defined herein. In certain embodiments, R^{5f} is —SR^{1a}, wherein R^{1a} is as defined herein. In certain embodiments, R^{5f} is S(O)R^{1a}, wherein R^{1a} is as defined herein. In certain embodiments, R^{5f} is S(O)₂R^{1a}, wherein R^{1a} is as defined herein. In certain embodiments, R^{5f} is S(O)NR^{1b}R^{1c}, wherein R^{1b} and R^{1c} are each as defined herein. In certain embodiments, R^{5f} is S(O)₂NR^{1b}R^{1c}; wherein R^{1b} and R^{1c} are each as defined herein.

In certain embodiments, R^{5g} is hydrogen. In certain embodiments, R^{5g} is halo. In certain embodiments, R^{5g} is fluoro, chloro, bromo, or iodo. In certain embodiments, R^{5g} is C₁₋₆ alkyl, optionally substituted with one, two, three, four, or five substituents Q as described herein. In certain embodiments, R^{5g} is methyl, optionally substituted with one, two, three, four, or five substituents Q as described herein. In certain embodiments, R^{5g} is methyl. In certain embodiments, R^{5g} is methyl, ethyl, propyl, or butyl, each optionally substituted with one, two, three, four, or five substituents Q as described herein. In certain embodiments, R^{5g} is methyl, ethyl, n-propyl, isopropyl, n-butyl, isobutyl, or t-butyl. In certain embodiments, R^{5g} is C₂₋₆ alkenyl, optionally substituted with one, two, three, four, or five substituents Q as described herein. In certain embodiments, R^{5g} is C₂₋₆ alkynyl, optionally substituted with one, two, three, four, or five substituents Q as described herein. In certain embodiments, R^{5g} is C₃₋₁₀ cycloalkyl, optionally substituted with one, two, three, four, or five substituents Q as described herein. In certain embodiments, R^{5g} is C₆₋₁₄ aryl, optionally substituted with one, two, three, four, or five substituents Q as described herein. In certain embodiments, R^{5g} is C₇₋₁₅ aralkyl, optionally substituted with one, two, three, four, or five substituents Q as described herein. In certain embodiments, R^{5g} is heteroaryl, optionally substituted with one, two, three, four, or five substituents Q as described herein. In certain embodiments, R^{5g} is heterocyclyl, optionally substituted with one, two, three, four, or five substituents Q as described herein.

In certain embodiments, R^{5g} is C(O)R^{1a}, wherein R^{1a} is as defined herein. In certain embodiments, R^{5g} is C(O)OR^{1a}, wherein R^{1a} is as defined herein. In certain embodiments, R^{5g} is —C(O)OR^{1a}, wherein R^{1a} is C₁₋₆ alkyl, optionally substituted with one, two, three, four, or five substituents Q as described herein. In certain embodiments, R^{5g} is C(O)OCH₃. In certain embodiments, R^{5g} is —C(O)NR^{1b}R^{1c}, wherein R^{1b} and R^{1c} are each as defined herein. In certain embodiments, R^{5g} is —C(NR^{1a})NR^{1b}R^{1c}, wherein R^{1a}, R^{1b}, and R^{1c} are each as defined herein. In certain embodiments, R^{5g} is —OR^{1a}, wherein R^{1a} is as defined herein. In certain embodiments, R^{5g} is —OC(O)R^{1a}, wherein R^{1a} is as defined herein. In certain embodiments, R^{5g} is —OC(O)OR^{1a}, wherein R^{1a} is as defined herein. In certain embodiments, R^{5g} is —OC(O)NR^{1b}R^{1c}, wherein R^{1b} and R^{1c} are each as defined herein. In certain embodiments, R^{5g} is —OC(═NR^{1a})NR^{1b}R^{1c}, wherein R^{1a}, R^{1b}, and R^{1c} are each as defined herein. In certain embodiments, R^{5g} is OS(O)R^{1a}, wherein R^{1a} is as defined herein. In certain embodiments, R^{5g} is —OS(O)₂R^{1a}, wherein R^{1a} is as defined herein. In certain embodiments, R^{5g} is OS(O)NR^{1b}R^{1c}, wherein R^{1b} and R^{1c} are each as defined herein. In certain embodiments, R^{5g} is OS(O)₂NR^{1b}R^{1c}, wherein R^{1b} and R^{1c} are each as defined herein. In certain embodiments, R^{5g} is —NR^{1b}R^{1c}, wherein R^{1b} and R^{1c} are each as defined herein. In certain embodiments, R^{5g} is amino (NH₂). In certain embodiments, R^{5g} is —NR^{1a}C(O)R^{1d}, wherein R^{1a} and R^{1d} are each as defined herein. In certain embodiments, R^{5g} is —NR^{1a}C(O)OR^{1d}, wherein R^{1a} and R^{1d} are each as defined herein. In certain embodiments, R^{5g} is —NR^{1a}C(O)NR^{1b}R^{1c}, wherein R^{1a}, R^{1b}, and R^{1c} are each as defined herein. In certain embodiments, R^{5g} is —NR^{1a}C(═NR^{1d})NR^{1b}R^{1c}, wherein R^{1a}, R^{1b}, R^{1c}, and R^{1d} are each as defined herein. In certain embodiments, R^{5g} is —NR^{1a}S(O)R^{1d}, wherein R^{1a} and R^{1d} are each as defined herein. In certain embodiments, R^{5g} is —NR^{1a}S(O)₂R^{1d}, wherein R^{1a} and R^{1d} are each as defined herein. In certain embodiments, R^{5g} is —NR^{1a}S(O)NR^{1b}R^{1c}, wherein R^{1a}, R^{1b}, and R^{1c} are each as defined herein. In certain embodiments, R^{5g} is —NR^{1a}S(O)₂NR^{1b}R^{1c}, wherein R^{1a}, R^{1b}, and R^{1c} are each as defined herein. In certain embodiments, R^{5g} is —SR^{1a}, wherein R^{1a} is as defined herein. In certain embodiments, R^{5g} is S(O)R^{1a}, wherein R^{1a} is as defined herein. In certain embodiments, R^{5g} is S(O)₂R^{1a}, wherein R^{1a} is as defined herein. In certain embodiments, R^{5g} is S(O)NR^{1b}R^{1c}, wherein R^{1b} and R^{1c} are each as defined herein. In certain embodiments, R^{5g} is S(O)₂NR^{1b}R^{1c}; wherein R^{1b} and R^{1c} are each as defined herein.

In certain embodiments, when one occurrence of R^{5f} and one occurrence of R^{5g} are attached to the same carbon atom, the R^{5f} and R^{5g} together with the carbon atom to which they are attached form a C₃₋₁₀ cycloalkyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a C₃₋₇ cycloalkyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a cyclopropyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a cyclobutyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a cyclopentyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a cyclohexyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a cycloheptyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a cyclopropyl.

In certain embodiments, when one occurrence of R^{5f} and one occurrence of R^{5g} are attached to the same carbon atom, the R^{5f} and R^{5g} together with the carbon atom to which they are attached form a heterocyclyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a 3-membered heterocyclyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a 4-membered heterocyclyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a 5-membered heterocyclyl, optionally substituted with one, two, three, four, or five substituents Q as described herein. In certain embodiments, when one occurrence of R^{5f} and one occurrence of R^{5g} are attached to the same carbon atom, the R^{5f} and R^{5g} together with the carbon atom to which they are attached form a 6-membered heterocyclyl, optionally substituted with one, two, three, four, or five substituents Q as described herein.

In certain embodiments, R^{7a} is hydrogen. In certain embodiments, R^{7a} is cyano. In certain embodiments, R^{7a} is halo. In certain embodiments, R^{7a} is fluoro, chloro, bromo, or iodo. In certain embodiments, R^{7a} is nitro. In certain embodiments, R^{7a} is C₁₋₆ alkyl, optionally substituted with one, two, three, or four substituents Q^{a} as described herein. In certain embodiments, R^{7a} is C₂₋₆ alkenyl, optionally substituted with one, two, three, or four substituents Q^{a} as described herein. In certain embodiments, R^{7a} is C₂₋₆ alkynyl, optionally substituted with one, two, three, or four substituents Q^{a} as described herein. In certain embodiments, R^{7a} is C₃₋₇ cycloalkyl, optionally substituted with one, two, three, or four substituents Q^{a} as described herein. In certain embodiments, R^{7a} is C₃₋₁₀ cycloalkyl, optionally substituted with one, two, three, or four substituents Q^{a} as described herein. In certain embodiments, R^{7a} is C₆₋₁₄ aryl, optionally substituted with one, two, three, or four substituents Q^{a} as described herein. In certain embodiments, R^{7a} is phenyl, optionally substituted with one, two, three, or four substituents Q^{a} as described herein. In certain embodiments, R^{7a} is phenyl, optionally substituted with one or more substituents, each of which is selected independently from the group consisting of fluoro, chloro, bromo, methyl, and methoxy. In certain embodiments, R^{7a} is phenyl, 2-fluorophenyl, 2-chlorophenyl, 2-bromophenyl, 2-methylphenyl, 2-methoxyphenyl, 3-fluorophenyl, 3-chlorophenyl, 3-methoxyphenyl, 4-fluorophenyl, 4-chlorophenyl, 4-bromophenyl, 4-methoxyphenyl. In certain embodiments, R^{7a} is C₇ aralkyl, optionally substituted with one, two, three, or four substituents Q^{a} as described herein. In certain embodiments, R^{7a} is heteroaryl, optionally substituted with one, two, three, or four substituents Q^{a} as described herein. In certain embodiments, R^{7a} is monocyclic heteroaryl, optionally substituted with one, two, three, or four substituents Q^{a} as described herein. In certain embodiments, R^{7a} is 5-membered heteroaryl, optionally substituted with one, two, three, or four substituents Q^{a} as described herein. In certain embodiments, R^{7a} is imidazolyl or pyrozolyl, optionally substituted with one, two, three, or four substituents Q^{a} as described herein. In certain embodiments, R^{7a} is imidazol-1-yl, pyrozol-4-yl, 1-methyl-pyrozol-4-yl, or 2-methylpyrozol-3-yl. In certain embodiments, R^{7a} is 6-membered heteroaryl, optionally substituted with one, two, three, or four substituents Q^{a} as described herein. In certain embodiments, R^{7a} is pyridinyl, optionally substituted with one, two, three, or four substituents Q^{a} as described herein. In certain embodiments, R^{7a} is pyridin-2-yl, pyridin-3-yl, pyridin-4-yl, 2-methylpyridin-4-yl, or 2-methoxypyridin-4-yl. In certain embodiments, R^{7a} is heterocyclyl, optionally substituted with one, two, three, or four substituents Q^{a} as described herein. In certain embodiments, R^{7a} is monocyclic heterocyclyl, optionally substituted with one, two, three, or four substituents Q^{a} as described herein. In certain embodiments, R^{7a} is 5-membered heterocyclyl, optionally substituted with one, two, three, or four substituents Q^{a} as described herein. In certain embodiments, R^{7a} is 6-membered heterocyclyl, optionally substituted with one, two, three, or four substituents Q^{a} as described herein. In certain embodiments, R^{7a} is piperidinyl or piperazinyl, optionally substituted with one, two, three, or four substituents Q^{a} as described herein. In certain embodiments, R^{7a} is 1-methylpiperidin-4-yl, or 4-methylpiperazin-1-yl.

In certain embodiments, R^{7a} is C(O)R^{a}, wherein R^{a} is as defined herein. In certain embodiments, R^{7a} is C(O)OR^{a}, wherein R^{a} is as defined herein. In certain embodiments, R^{7a} is C(O)NR^{b}R^{c}, wherein R^{b} and R^{c} are each as defined herein. In certain embodiments, R^{7a} is C(NR^{a})NR^{b}R^{c}, wherein R^{a}, R^{b}, and R^{c} are each as defined herein. In certain embodiments, R^{7a} is OR^{a}, wherein R^{a} is as defined herein. In certain embodiments, R^{a} is —O—C₁₋₆ alkyl, wherein the alkyl is optionally substituted with one, two, three, or four substituents Q^{a} as described herein. In certain embodiments, R^{a} is methoxy, ethoxy, propoxy, isopropoxy, or 3-dimethylaminopropoxy. In certain embodiments, R^{7a} is —OC(O)R^{a}, wherein R^{a} is as defined herein. In certain embodiments, R^{7a} is —OC(O)OR^{a}, wherein R^{a} is as defined herein. In certain embodiments, R^{7a} is —OC(O)NR^{b}R^{c}, wherein R^{b} and R^{c} are each as defined herein. In certain embodiments, R^{7a} is —OC(═NR^{a})NR^{b}R^{c}, wherein R^{a}, R^{b}, and R^{c} are each as defined herein. In certain embodiments, R^{7a} is —OS(O)R^{a}, wherein R^{a} is as defined herein. In certain embodiments, R^{7a} is —OS(O)₂R^{a}, wherein R^{a} is as defined herein. In certain embodiments, R^{7a} is —OS(O)NR^{b}R^{c}, wherein R^{b} and R^{c} are each as defined herein. In certain embodiments, R^{7a} is —OS(O)₂NR^{b}R^{c}, wherein R^{b} and R^{c} are each as defined herein. In certain embodiments, R^{7a} is —NR^{b}R^{c}, wherein R^{b} and R^{c} are each as defined herein. In certain embodiments, R^{7a} is amino (—NH₂). In certain embodiments, R^{7a} is —NR^{a}C(O)R^{d}, wherein R^{a} and R^{d} are each as defined herein. In certain embodiments, R^{7a} is —NR^{a}C(O)OR^{d}, wherein R^{a} and R^{d} are each as defined herein. In certain embodiments, R^{7a} is —NR^{a}C(O)NR^{b}R^{c}, wherein R^{a}, R^{b}, and R^{c} are each as defined herein. In certain embodiments, R^{7a} is —NR^{a}C(═NR^{d})NR^{b}R^{c}, wherein R^{a}, R^{b}, R^{c}, and R^{d} are each as defined herein. In certain embodiments, R^{7a} is —NR^{a}S(O)R^{d}, wherein R^{a} and R^{d} are each as defined herein. In certain embodiments, R^{7a} is —NR^{a}S(O)₂R^{d}, wherein R^{a} and R^{d} are each as defined herein. In certain embodiments, R^{7a} is —NR^{a}S(O)NR^{b}R^{c}, wherein R^{a}, R^{b}, and R^{c} are each as defined herein. In certain embodiments, R^{7a} is —NR^{a}S(O)₂NR^{b}R^{c}, wherein R^{a}, R^{b}, and R^{c} are each as defined herein. In certain embodiments, R^{7a} is —SR^{a}, wherein R^{a} is as defined herein. In certain embodiments, R^{7a} is —S(O)R^{a}, wherein R^{a} is as defined herein. In certain embodiments, R^{7a} is —S(O)₂R^{a}, wherein R^{a} is as defined herein. In certain embodiments, R^{7a} is —S(O)NR^{b}R^{c}, wherein R^{b} and R^{c} are each as defined herein. In certain embodiments, R^{7a} is —S(O)₂NR^{b}R^{c}; wherein R^{b} and R^{c} are each as defined herein.

In certain embodiments, R^{7a} is phenyl, imidazolyl, pyrozolyl, pyridinyl, pyrimidinyl, pyrrolidinyl, piperidinyl, or piperazinyl, each optionally substituted with one, two, three, or four substituents Q^{a}. In certain embodiments, R^{7a} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments, R^{7b} is hydrogen. In certain embodiments, R^{7b} is cyano. In certain embodiments, R^{7b} is halo. In certain embodiments, R^{7b} is fluoro, chloro, bromo, or iodo. In certain embodiments, R^{7b} is nitro. In certain embodiments, R^{7b} is C₁₋₆ alkyl, optionally substituted with one, two, three, or four substituents Q^{a} as described herein. In certain embodiments, R^{7b} is C₂₋₆ alkenyl, optionally substituted with one, two, three, or four substituents Q^{a} as described herein. In certain embodiments, R^{7b} is C₂₋₆ alkynyl, optionally substituted with one, two, three, or four substituents Q^{a} as described herein. In certain embodiments, R^{7b} is C₃₋₁₀ cycloalkyl, optionally substituted with one, two, three, or four substituents Q^{a} as described herein. In certain embodiments, R^{7b} is C₃₋₇ cycloalkyl, optionally substituted with one, two, three, or four substituents Q^{a} as described herein. In certain embodiments, R^{7b} is C₆₋₁₄ aryl, optionally substituted with one, two, three, or four substituents Q^{a} as described herein. In certain embodiments, R^{7b} is C₇₋₁₅ aralkyl, optionally substituted with one, two, three, or four substituents Q^{a} as described herein. In certain embodiments, R^{7b} is heteroaryl, optionally substituted with one, two, three, or four substituents Q^{a} as described herein. In certain embodiments, R^{7b} is heterocyclyl, optionally substituted with one, two, three, or four substituents Q^{a} as described herein.

In certain embodiments, R^{7b} is —C(O)R^{a}, wherein R^{a} is as defined herein. In certain embodiments, R^{7b} is —C(O)OR^{a}, wherein R^{a} is as defined herein. In certain embodiments, R^{7b} is —C(O)NR^{b}R^{c}, wherein R^{b} and R^{c} are each as defined herein. In certain embodiments, R^{7b} is —C(NR^{a})NR^{b}R^{c}, wherein R^{a}, R^{b}, and R^{c} are each as defined herein. In certain embodiments, R^{7b} is —OR^{a}, wherein R^{a} is as defined herein. In certain embodiments, R^{a} is —O—C₁₋₆ alkyl, wherein the alkyl is optionally substituted with one, two, three, or four substituents Q^{a} as described herein. In certain embodiments, R^{a} is methoxy, ethoxy, propoxy, isopropoxy, or 3-dimethylaminopropoxy. In certain embodiments, R^{7b} is —OC(O)R^{a}, wherein R^{a} is as defined herein. In certain embodiments, R^{7b} is —OC(O)OR^{a}, wherein R^{a} is as defined herein. In certain embodiments, R^{7b} is —OC(O)NR^{b}R^{c}, wherein R^{b} and R^{c} are each as defined herein. In certain embodiments, R^{7b} is —OC(═NR^{a})NR^{b}R^{c}, wherein R^{a}, R^{b}, and R^{c} are each as defined herein. In certain embodiments, R^{7b} is —OS(O)R^{a}, wherein R^{a} is as defined herein. In certain embodiments, R^{7b} is —OS(O)₂R^{a}, wherein R^{a} is as defined herein. In certain embodiments, R^{7b} is —OS(O)NR^{b}R^{c}, wherein R^{b} and R^{c} are each as defined herein. In certain embodiments, R^{7b} is —OS(O)₂NR^{b}R^{c}, wherein R^{b} and R^{c} are each as defined herein. In certain embodiments, R^{7b} is —NR^{b}R^{c}, wherein R^{b} and R^{c} are each as defined herein. In certain embodiments, R^{7b} is amino (—NH₂). In certain embodiments, R^{7b} is —NR^{a}C(O)R^{d}, wherein R^{a} and R^{d} are each as defined herein. In certain embodiments, R^{7b} is —NR^{a}C(O)OR^{d}, wherein R^{a} and R^{d} are each as defined herein. In certain embodiments, R^{7b} is —NR^{a}C(O)NR^{b}R^{c}, wherein R^{a}, R^{b}, and R^{c} are each as defined herein. In certain embodiments, R^{7b} is —NR^{a}C(═NR^{d})NR^{b}R^{c}, wherein R^{a}, R^{b}, R^{c}, and R^{d} are each as defined herein. In certain embodiments, R^{7b} is —NR^{a}S(O)R^{d}, wherein R^{a} and R^{d} are each as defined herein. In certain embodiments, R^{7b} is —NR^{a}S(O)₂R^{d}, wherein R^{a} and R^{d} are each as defined herein. In certain embodiments, R^{7b} is —NR^{a}S(O)NR^{b}R^{c}, wherein R^{a}, R^{b}, and R^{c} are each as defined herein. In certain embodiments, R^{7b} is —NR^{a}S(O)₂NR^{b}R^{c}, wherein R^{a}, R^{b}, and R^{c} are each as defined herein. In certain embodiments, R^{7b} is —SR^{a}, wherein R^{a} is as defined herein. In certain embodiments, R^{7b} is —S(O)R^{a}, wherein R^{a} is as defined herein. In certain embodiments, R^{7b} is —S(O)₂R^{a}, wherein R^{a} is as defined herein. In certain embodiments, R^{7b} is —S(O)NR^{b}R^{c}, wherein R^{b} and R^{c} are each as defined herein. In certain embodiments, R^{7b} is —S(O)₂NR^{b}R^{c}; wherein R^{b} and R^{c} are each as defined herein.

In certain embodiments, R^{7b} is phenyl, imidazolyl, pyrozolyl, pyridinyl, pyrimidinyl, pyrrolidinyl, piperidinyl, or piperazinyl, each optionally substituted with one, two, three, or four substituents Q^{a}. In certain embodiments, R^{7b} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments, R^{7c} is hydrogen. In certain embodiments, R^{7c} is cyano. In certain embodiments, R^{7c} is halo. In certain embodiments, R^{7c} is fluoro, chloro, bromo, or iodo. In certain embodiments, R^{7c} is nitro. In certain embodiments, R^{7c} is C₁₋₆ alkyl, optionally substituted with one, two, three, or four substituents Q^{a} as described herein. In certain embodiments, R^{7c} is C₂₋₆ alkenyl, optionally substituted with one, two, three, or four substituents Q^{a} as described herein. In certain embodiments, R^{7c} is C₂₋₆ alkynyl, optionally substituted with one, two, three, or four substituents Q^{a} as described herein. In certain embodiments, R^{7c} is C₃₋₁₀ cycloalkyl, optionally substituted with one, two, three, or four substituents Q^{a} as described herein. In certain embodiments, R^{7c} is C₃₋₇ cycloalkyl, optionally substituted with one, two, three, or four substituents Q^{a} as described herein. In certain embodiments, R^{7c} is C₆₋₁₄ aryl, optionally substituted with one, two, three, or four substituents Q^{a} as described herein. In certain embodiments, R^{7c} is C₇₋₁₅ aralkyl, optionally substituted with one, two, three, or four substituents Q^{a} as described herein. In certain embodiments, R^{7c} is heteroaryl, optionally substituted with one, two, three, or four substituents Q^{a} as described herein. In certain embodiments, R^{7c} is heterocyclyl, optionally substituted with one, two, three, or four substituents Q^{a} as described herein.

In certain embodiments, R^{7c} is C(O)R^{d}, wherein R^{a} is as defined herein. In certain embodiments, R^{7c} is C(O)OR^{a}, wherein R^{a} is as defined herein. In certain embodiments, R^{7c} is C(O)NR^{b}R^{c}, wherein R^{b} and TIC are each as defined herein. In certain embodiments, R^{7c} is C(NR^{a})NR^{b}R^{c}, wherein R^{a}, R^{b}, and R^{c} are each as defined herein. In certain embodiments, R^{7c} is OR^{d}, wherein R^{a} is as defined herein. In certain embodiments, R^{a} is —OC₁₋₆ alkyl, wherein the alkyl is optionally substituted with one, two, three, or four substituents Q^{a} as described herein. In certain embodiments, R^{a} is methoxy, ethoxy, propoxy, isopropoxy, or 3-dimethylaminopropoxy. In certain embodiments, R^{7c} is —OC(O)R^{d}, wherein R^{a} is as defined herein. In certain embodiments, R^{7c} is —OC(O)OR^{a}, wherein R^{a} is as defined herein. In certain embodiments, R^{7c} is —OC(O)NR^{b}R^{c}, wherein R^{b} and R^{c} are each as defined herein. In certain embodiments, R^{7c} is —OC(═NR^{a})NR^{b}R^{c}, wherein R^{a}, R^{b}, and R^{c} are each as defined herein. In certain embodiments, R^{7c} is —OS(O)R^{a}, wherein R^{a} is as defined herein. In certain embodiments, R^{7c} is OS(O)₂R^{d}, wherein R^{a} is as defined herein. In certain embodiments, R^{7c} is OS(O)NR^{b}R^{c}, wherein R^{b} and R^{c} are each as defined herein. In certain embodiments, R^{7c} is OS(O)₂NR^{b}R^{c}, wherein R^{b} and R^{c} are each as defined herein. In certain embodiments, R^{7c} is —NR^{b}R^{c}, wherein R^{b} and R^{c} are each as defined herein. In certain embodiments, R^{7c} is amino (NH₂). In certain embodiments, R^{7c} is —NR^{a}C(O)R^{d}, wherein R^{a} and R^{d} are each as defined herein. In certain embodiments, R^{7c} is —NR^{a}C(O)OR^{d}, wherein R^{a} and R^{d} are each as defined herein. In certain embodiments, R^{7c} is —NR^{a}C(O)NR^{b}R^{c}, wherein R^{a}, R^{b}, and R^{c} are each as defined herein. In certain embodiments, R^{7c} is —NR^{a}C(═NR^{d})NR^{b}R^{c}, wherein R^{a}, R^{b}, R^{c}, and R^{d} are each as defined herein. In certain embodiments, R^{7c} is —NR^{a}S(O)R^{d}, wherein R^{a} and R^{d} are each as defined herein. In certain embodiments, R^{7c} is —NR^{a}S(O)₂R^{d}, wherein R^{a} and R^{d} are each as defined herein. In certain embodiments, R^{7c} is —NR^{a}S(O)NR^{b}R^{c}, wherein R^{a}, R^{b}, and R^{c} are each as defined herein. In certain embodiments, R^{7c} is —NR^{a}S(O)₂NR^{b}R^{c}, wherein R^{a}, R^{b}, and R^{c} are each as defined herein. In certain embodiments, R^{7c} is —SR^{a}, wherein R^{a} is as defined herein. In certain embodiments, R^{7c} is —S(O)R^{a}, wherein R^{a} is as defined herein. In certain embodiments, R^{7c} is —S(O)₂R^{a}, wherein R^{a} is as defined herein. In certain embodiments, R^{7c} is —S(O)NR^{b}R^{c}, wherein R^{b} and R^{c} are each as defined herein. In certain embodiments, R^{7c} is —S(O)₂NR^{b}R^{c}; wherein R^{b} and R^{c} are each as defined herein.

In certain embodiments, R^{7c} is phenyl, imidazolyl, pyrozolyl, pyridinyl, pyrimidinyl, pyrrolidinyl, piperidinyl, or piperazinyl, each optionally substituted with one, two, three, or four substituents Q^{a}. In certain embodiments, R^{7c} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments, R^{7d} is hydrogen. In certain embodiments, R^{7d} is cyano. In certain embodiments, R^{7d} is halo. In certain embodiments, R^{7d} is fluoro, chloro, bromo, or iodo. In certain embodiments, R^{7d} is nitro. In certain embodiments, R^{7d} is C₁₋₆ alkyl, optionally substituted with one, two, three, or four substituents Q^{a} as described herein. In certain embodiments, R^{7d} is C₂₋₆ alkenyl, optionally substituted with one, two, three, or four substituents Q^{a} as described herein. In certain embodiments, R^{7d} is C₂₋₆ alkynyl, optionally substituted with one, two, three, or four substituents Q^{a} as described herein. In certain embodiments, R^{7d} is C₃₋₁₀ cycloalkyl, optionally substituted with one, two, three, or four substituents Q^{a} as described herein. In certain embodiments, R^{7d} is C₃₋₇ cycloalkyl, optionally substituted with one, two, three, or four substituents Q^{a} as described herein. In certain embodiments, R^{7d} is C₆₋₁₄ aryl, optionally substituted with one, two, three, or four substituents Q^{a} as described herein. In certain embodiments, R^{7d} is C₇₋₁₅ aralkyl, optionally substituted with one, two, three, or four substituents Q^{a} as described herein. In certain embodiments, R^{7d} is heteroaryl, optionally substituted with one, two, three, or four substituents Q^{a} as described herein. In certain embodiments, R^{7d} is heterocyclyl, optionally substituted with one, two, three, or four substituents Q^{a} as described herein.

In certain embodiments, R^{7d} is —C(O)R^{a}, wherein R^{a} is as defined herein. In certain embodiments, R^{7d} is —C(O)OR^{a}, wherein R^{a} is as defined herein. In certain embodiments, R^{7d} is —C(O)NR^{b}R^{c}, wherein R^{b} and R^{c} are each as defined herein. In certain embodiments, R^{7d} is —C(NR^{a})NR^{b}R^{c}, wherein R^{a}, R^{b}, and R^{c} are each as defined herein. In certain embodiments, R^{7d} is —OR^{d}, wherein R^{a} is as defined herein. In certain embodiments, R^{a} is —O—C₁₋₆ alkyl, wherein the alkyl is optionally substituted with one, two, three, or four substituents Q^{a} as described herein. In certain embodiments, R^{a} is methoxy, ethoxy, propoxy, isopropoxy, or 3-dimethylaminopropoxy. In certain embodiments, R^{7d} is —OC(O)R^{a}, wherein R^{a} is as defined herein. In certain embodiments, R^{7d} is —OC(O)OR^{a}, wherein R^{a} is as defined herein. In certain embodiments, R^{7d} is —OC(O)NR^{b}R^{c}, wherein R^{b} and R^{c} are each as defined herein. In certain embodiments, R^{7d} is —OC(═NR^{a})NR^{b}R^{c}, wherein R^{a}, R^{b}, and R^{c} are each as defined herein. In certain embodiments, R^{7d} is —OS(O)R^{a}, wherein R^{a} is as defined herein. In certain embodiments, R^{7d} is —OS(O)₂R^{a}, wherein R^{a} is as defined herein. In certain embodiments, R^{7d} is —OS(O)NR^{b}R^{c}, wherein R^{b} and TIC are each as defined herein. In certain embodiments, R^{7d} is —OS(O)₂NR^{b}R^{c}, wherein R^{b} and R^{c} are each as defined herein. In certain embodiments, R^{7d} is —NR^{b}R^{c}, wherein R^{b} and TIC are each as defined herein. In certain embodiments, R^{7d} is amino (—NH₂). In certain embodiments, R^{7d} is —NR^{a}C(O)R^{d}, wherein R^{a} and R^{d} are each as defined herein. In certain embodiments, R^{7d} is —NR^{a}C(O)OR^{d}, wherein R^{a} and R^{d} are each as defined herein. In certain embodiments, R^{7d} is —NR^{a}C(O)NR^{b}R^{c}, wherein R^{a}, R^{b}, and R^{c} are each as defined herein. In certain embodiments, R^{7d} is —NR^{a}C(═NR^{d})NR^{b}R^{c}, wherein R^{a}, R^{b}, R^{c}, and R^{d} are each as defined herein. In certain embodiments, R^{7d} is —NR^{a}S(O)R^{d}, wherein R^{a} and R^{d} are each as defined herein. In certain embodiments, R^{7d} is —NR^{a}S(O)₂R^{d}, wherein R^{a} and R^{d} are each as defined herein. In certain embodiments, R^{7d} is —NR^{a}S(O)NR^{b}R^{c}, wherein R^{a}, R^{b}, and R^{c} are each as defined herein. In certain embodiments, R^{7d} is —NR^{a}S(O)₂NR^{b}R^{c}, wherein R^{a}, R^{b}, and R^{c} are each as defined herein. In certain embodiments, R^{7d} is —SR^{a}, wherein R^{a} is as defined herein. In certain embodiments, R^{7d} is —S(O)R^{a}, wherein R^{a} is as defined herein. In certain embodiments, R^{7d} is —S(O)₂R^{a}, wherein R^{a} is as defined herein. In certain embodiments, R^{7d} is —S(O)NR^{b}R^{c}, wherein R^{b} and R^{c} are each as defined herein. In certain embodiments, R^{7d} is —S(O)₂NR^{b}R^{c}; wherein R^{b} and R^{c} are each as defined herein.

In certain embodiments, R^{7d} is phenyl, imidazolyl, pyrozolyl, pyridinyl, pyrimidinyl, pyrrolidinyl, piperidinyl, or piperazinyl, each optionally substituted with one, two, three, or four substituents Q^{a}. In certain embodiments, R^{7d} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments, R^{7e} is hydrogen. In certain embodiments, R^{7e} is cyano. In certain embodiments, R^{7e} is halo. In certain embodiments, R^{7c} is fluoro, chloro, bromo, or iodo. In certain embodiments, R^{7e} is nitro. In certain embodiments, R^{7e} is C₁₋₆ alkyl, optionally substituted with one, two, three, or four substituents Q^{a} as described herein. In certain embodiments, R^{7c} is C₂₋₆ alkenyl, optionally substituted with one, two, three, or four substituents Q^{a} as described herein. In certain embodiments, R^{7e} is C₂₋₆ alkynyl, optionally substituted with one, two, three, or four substituents Q^{a} as described herein. In certain embodiments, R^{7e} is C₃₋₁₀ cycloalkyl, optionally substituted with one, two, three, or four substituents Q^{a} as described herein. In certain embodiments, R^{7e} is C₃₋₇ cycloalkyl, optionally substituted with one, two, three, or four substituents Q^{a} as described herein. In certain embodiments, R^{7e} is C₆₋₁₄ aryl, optionally substituted with one, two, three, or four substituents Q^{a} as described herein. In certain embodiments, R^{7e} is C₇₋₁₅ aralkyl, optionally substituted with one, two, three, or four substituents Q^{a} as described herein. In certain embodiments, R^{7e} is heteroaryl, optionally substituted with one, two, three, or four substituents Q^{a} as described herein. In certain embodiments, R^{7e} is heterocyclyl, optionally substituted with one, two, three, or four substituents Q^{a} as described herein.

In certain embodiments, R^{7e} is C(O)R^{a}, wherein R^{a} is as defined herein. In certain embodiments, R^{7e} is C(O)OR^{a}, wherein R^{a} is as defined herein. In certain embodiments, R^{7e} is C(O)NR^{b}R^{c}, wherein R^{b} and R^{c} are each as defined herein. In certain embodiments, R^{7e} is C(NR^{a})NR^{b}R^{c}, wherein R^{a}, R^{b}, and R^{c} are each as defined herein. In certain embodiments, R^{7e} is OR^{a}, wherein R^{a} is as defined herein. In certain embodiments, R^{a} is —OC₁₋₆ alkyl, wherein the alkyl is optionally substituted with one, two, three, or four substituents Q^{a} as described herein. In certain embodiments, R^{a} is methoxy, ethoxy, propoxy, isopropoxy, or 3-dimethylaminopropoxy. In certain embodiments, R^{7e} is —OC(O)R^{a}, wherein R^{a} is as defined herein. In certain embodiments, R^{7e} is —OC(O)OR^{a}, wherein R^{a} is as defined herein. In certain embodiments, R^{7e} is —OC(O)NR^{b}R^{c}, wherein R^{b} and R^{c} are each as defined herein. In certain embodiments, R^{7e} is —OC(═NR^{a})NR^{b}R^{c}, wherein R^{a}, R^{b}, and R^{c} are each as defined herein. In certain embodiments, R^{7e} is —OS(O)R^{a}, wherein R^{a} is as defined herein. In certain embodiments, R^{7e} is OS(O)₂R^{a}, wherein R^{a} is as defined herein. In certain embodiments, R^{7e} is OS(O)NR^{b}R^{c}, wherein R^{b} and R^{c} are each as defined herein. In certain embodiments, R^{7e} is OS(O)₂NR^{b}R^{c}, wherein R^{b} and R^{c} are each as defined herein. In certain embodiments, R^{7e} is —NR^{b}R^{c}, wherein R^{b} and R^{c} are each as defined herein. In certain embodiments, R^{7e} is amino (NH₂). In certain embodiments, R^{7e} is NR^{a}C(O)R^{d}, wherein R^{a} and R^{d} are each as defined herein. In certain embodiments, R^{7e} is —NR^{a}C(O)OR^{d}, wherein R^{a} and R^{d} are each as defined herein. In certain embodiments, R^{7e} is —NR^{a}C(O)NR^{b}R^{c}, wherein R^{a}, R^{b}, and R^{c} are each as defined herein. In certain embodiments, R^{7e} is —NR^{a}C(═NR^{d})NR^{b}R^{c}, wherein R^{a}, R^{b}, R^{c}, and R^{d} are each as defined herein. In certain embodiments, R^{7e} is —NR^{a}S(O)R^{d}, wherein R^{a} and R^{d} are each as defined herein. In certain embodiments, R^{7e} is —NR^{a}S(O)₂R^{d}, wherein R^{a} and R^{d} are each as defined herein. In certain embodiments, R^{7e} is —NR^{a}S(O)NR^{b}R^{c}, wherein R^{a}, R^{b}, and R^{c} are each as defined herein. In certain embodiments, R^{7e} is —NR^{a}S(O)₂NR^{b}R^{c}, wherein R^{a}, R^{b}, and R^{c} are each as defined herein. In certain embodiments, R^{7e} is —SR^{a}, wherein R^{a} is as defined herein. In certain embodiments, R^{7e} is S(O)R^{a}, wherein R^{a} is as defined herein. In certain embodiments, R^{7e} is S(O)₂R^{a}, wherein R^{a} is as defined herein. In certain embodiments, R^{7e} is S(O)NR^{b}R^{c}, wherein R^{b} and R^{c} are each as defined herein. In certain embodiments, R^{7e} is S(O)₂NR^{b}R^{c}; wherein R^{b} and R^{c} are each as defined herein.

In certain embodiments, R^{7e} is phenyl, imidazolyl, pyrozolyl, pyridinyl, pyrimidinyl, pyrrolidinyl, piperidinyl, or piperazinyl, each optionally substituted with one, two, three, or four substituents Q^{a}. In certain embodiments, R^{7e} is phenyl, 2-fluorophenyl, 2-chlorophenyl, 2-bromophenyl, 2-methylphenyl, 2-(3-dimethylaminopropyl)phenyl, 2-methoxyphenyl, 3-fluorophenyl, 3-chlorophenyl, 3-methylphenyl, 3-methoxyphenyl, 4-fluorophenyl, 4-chlorophenyl, 4-bromophenyl, 4-methoxyphenyl, 2,4-difluorophenyl, 2,6-difluorophenyl, 4-fluoro-3-methoxyphenyl, 3-methoxyphenyl, 4-methoxyphenyl, 3-morpholin-4-ylmethylphenyl, imidazol-1-yl, pyrozol-4-yl, 1-methyl-pyrozol-4-yl, 2-methylpyrozol-3-yl, pyridin-2-yl, pyridin-3-yl, pyridin-4-yl, 2-fluoropyridin-3-yl, 2-methylpyridin-4-yl, 2-(4-methylpiperazin-1-yl)pyridin-4-yl, 2-methoxypyridin-4-yl, pyrimidin-5-yl, pyrrolidin-3-yl, 1-methylpyrrolidin-3-yl, piperidin-4-yl, 1-methylpiperidin-4-yl, 1-ethylpiperidin-4-yl, 1-isopropylpiperidin-4-yl, 1-acetylpiperidin-4-yl, 1-methylsulfonylpiperidin-4-yl, or 4-methylpiperazin-1-yl.

In certain embodiments, R^{7a} and R^{7b} together with the carbon atoms to which they are attached form C₃₋₁₀ cycloalkenyl, C₆₋₁₄ aryl, heteroaryl, or heterocyclyl, each optionally substituted with one, two, three, or four substituents Q^{a}. In certain embodiments, R^{7a} and R^{7b} together with the carbon atoms to which they are attached form C₃₋₁₀ cycloalkenyl, optionally substituted with one, two, three, or four substituents Q^{a}. In certain embodiments, R^{7a} and R^{7b} together with the carbon atoms to which they are attached form cyclohexenyl, optionally substituted with one, two, three, or four substituents Q^{a}. In certain embodiments, R^{7a} and R^{7b} together with the carbon atoms to which they are attached form C₆₋₁₄ aryl, optionally substituted with one, two, three, or four substituents Q^{a}. In certain embodiments, R^{7a} and R^{7b} together with the carbon atoms to which they are attached form phenyl, optionally substituted with one, two, three, or four substituents Q^{a}. In certain embodiments, R^{7a} and R^{7b} together with the carbon atoms to which they are attached form heteroaryl, optionally substituted with one, two, three, or four substituents Q^{a}. In certain embodiments, R^{7a} and R^{7b} together with the carbon atoms to which they are attached form monocyclic heteroaryl, optionally substituted with one, two, three, or four substituents Q^{a}. In certain embodiments, R^{7a} and R^{7b} together with the carbon atoms to which they are attached form 5- or 6-membered heteroaryl, optionally substituted with one, two, three, or four substituents Q. In certain embodiments, R^{7a} and R^{7b} together with the carbon atoms to which they are attached form bicyclic heteroaryl, optionally substituted with one, two, three, or four substituents Q^{a}. In certain embodiments, R^{7a} and R^{7b} together with the carbon atoms to which they are attached form heterocyclyl, optionally substituted with one, two, three, or four substituents Q^{a}. In certain embodiments, R^{7a} and R^{7b} together with the carbon atoms to which they are attached form monocyclic heterocyclyl, optionally substituted with one, two, three, or four substituents Q^{a}. In certain embodiments, R^{7a} and R^{7b} together with the carbon atoms to which they are attached form 5- or 6-membered heterocyclyl, optionally substituted with one, two, three, or four substituents Q^{a}. In certain embodiments, R^{7a} and R^{7b} together with the carbon atoms to which they are attached form bicyclic heterocyclyl, optionally substituted with one, two, three, or four substituents Q^{a}.

In certain embodiments, R^{7b} and R^{7c} together with the carbon atoms to which they are attached form C₃₋₁₀ cycloalkenyl, C₆₋₁₄ aryl, heteroaryl, or heterocyclyl, each optionally substituted with one, two, three, or four substituents Q^{a}. In certain embodiments, R^{7b} and R^{7c} together with the carbon atoms to which they are attached form C₃₋₁₀ cycloalkenyl, optionally substituted with one, two, three, or four substituents Q^{a}. In certain embodiments, R^{7b} and R^{7c} together with the carbon atoms to which they are attached form cyclohexenyl, optionally substituted with one, two, three, or four substituents Q^{a}. In certain embodiments, R^{7b} and R^{7c} together with the carbon atoms to which they are attached form C₆₋₁₄ aryl, optionally substituted with one, two, three, or four substituents Q^{a}. In certain embodiments, R^{7b} and R^{7c} together with the carbon atoms to which they are attached form phenyl, optionally substituted with one, two, three, or four substituents Q^{a}. In certain embodiments, R^{7b} and R^{7c} together with the carbon atoms to which they are attached form heteroaryl, optionally substituted with one, two, three, or four substituents Q^{a}. In certain embodiments, R^{7b} and R^{7c} together with the carbon atoms to which they are attached form monocyclic heteroaryl, optionally substituted with one, two, three, or four substituents Q^{a}. In certain embodiments, R^{7b} and R^{7c} together with the carbon atoms to which they are attached form 5- or 6-membered heteroaryl, optionally substituted with one, two, three, or four substituents Q^{a}. In certain embodiments, R^{7b} and R^{7c} together with the carbon atoms to which they are attached form bicyclic heteroaryl, optionally substituted with one, two, three, or four substituents Q^{a}. In certain embodiments, R^{7b} and R^{7c} together with the carbon atoms to which they are attached form heterocyclyl, optionally substituted with one, two, three, or four substituents Q^{a}. In certain embodiments, R^{7b} and R^{7c} together with the carbon atoms to which they are attached form monocyclic heterocyclyl, optionally substituted with one, two, three, or four substituents Q^{a}. In certain embodiments, R^{7b} and R^{7c} together with the carbon atoms to which they are attached form 5- or 6-membered heterocyclyl, optionally substituted with one, two, three, or four substituents Q^{a}. In certain embodiments, R^{7b} and R^{7c} together with the carbon atoms to which they are attached form bicyclic heterocyclyl, optionally substituted with one, two, three, or four substituents Q^{a}.

In certain embodiments, R^{7c} and R^{7d} together with the carbon atoms to which they are attached form C₃₋₁₀ cycloalkenyl, C₆₋₁₄ aryl, heteroaryl, or heterocyclyl, each optionally substituted with one, two, three, or four substituents Q^{a}. In certain embodiments, R^{7c} and R^{7d} together with the carbon atoms to which they are attached form C₃₋₁₀ cycloalkenyl, optionally substituted with one, two, three, or four substituents Q^{a}. In certain embodiments, R^{7c} and R^{7d} together with the carbon atoms to which they are attached form cyclohexenyl, optionally substituted with one, two, three, or four substituents Q^{a}. In certain embodiments, R^{7c} and R^{7d} together with the carbon atoms to which they are attached form C₆₋₁₄ aryl, optionally substituted with one, two, three, or four substituents Q^{a}. In certain embodiments, R^{7c} and R^{7b} together with the carbon atoms to which they are attached form phenyl, optionally substituted with one, two, three, or four substituents Q^{a}. In certain embodiments, R^{7c} and R^{7d} together with the carbon atoms to which they are attached form heteroaryl, optionally substituted with one, two, three, or four substituents Q^{a}. In certain embodiments, R^{7c} and R^{7d} together with the carbon atoms to which they are attached form monocyclic heteroaryl, optionally substituted with one, two, three, or four substituents Q^{a}. In certain embodiments, R^{7c} and R^{7d} together with the carbon atoms to which they are attached form 5- or 6-membered heteroaryl, optionally substituted with one, two, three, or four substituents Q^{a}. In certain embodiments, R^{7c} and R^{7d} together with the carbon atoms to which they are attached form bicyclic heteroaryl, optionally substituted with one, two, three, or four substituents Q^{a}. In certain embodiments, R^{7c} and R^{7d} together with the carbon atoms to which they are attached form heterocyclyl, optionally substituted with one, two, three, or four substituents Q^{a}. In certain embodiments, R^{7c} and R^{7d} together with the carbon atoms to which they are attached form monocyclic heterocyclyl, optionally substituted with one, two, three, or four substituents Q^{a}. In certain embodiments, R^{7c} and R^{7d} together with the carbon atoms to which they are attached form 5- or 6-membered heterocyclyl, optionally substituted with one, two, three, or four substituents Q^{a}. In certain embodiments, R^{7c} and R^{7d} together with the carbon atoms to which they are attached form bicyclic heterocyclyl, optionally substituted with one, two, three, or four substituents Q^{a}.

In certain embodiments, R^{7d} and R^{7e} together with the carbon atoms to which they are attached form C₃₋₁₀ cycloalkenyl, C₆₋₁₄ aryl, heteroaryl, or heterocyclyl, each optionally substituted with one, two, three, or four substituents Q^{a}. In certain embodiments, R^{7d} and R^{7e} together with the carbon atoms to which they are attached form C₃₋₁₀ cycloalkenyl, optionally substituted with one, two, three, or four substituents Q^{a}. In certain embodiments, R^{7d} and R^{7e} together with the carbon atoms to which they are attached form cyclohexenyl, optionally substituted with one, two, three, or four substituents Q^{a}. In certain embodiments, R^{7d} and R^{7e} together with the carbon atoms to which they are attached form C₆₋₁₄ aryl, optionally substituted with one, two, three, or four substituents Q^{a}. In certain embodiments, R^{7d} and R^{7e} together with the carbon atoms to which they are attached form phenyl, optionally substituted with one, two, three, or four substituents Q^{a}. In certain embodiments, R^{7d} and R^{7e} together with the carbon atoms to which they are attached form heteroaryl, optionally substituted with one, two, three, or four substituents Q^{a}. In certain embodiments, R^{7d} and R^{7e} together with the carbon atoms to which they are attached form monocyclic heteroaryl, optionally substituted with one, two, three, or four substituents Q^{a}. In certain embodiments, R^{7d} and R^{7e} together with the carbon atoms to which they are attached form 5- or 6-membered heteroaryl, optionally substituted with one, two, three, or four substituents Q^{a}. In certain embodiments, R^{7d} and R^{7e} together with the carbon atoms to which they are attached form bicyclic heteroaryl, optionally substituted with one, two, three, or four substituents Q^{a}. In certain embodiments, R^{7d} and R^{7e} together with the carbon atoms to which they are attached form heterocyclyl, optionally substituted with one, two, three, or four substituents Q^{a}. In certain embodiments, R^{7d} and R^{7e} together with the carbon atoms to which they are attached form monocyclic heterocyclyl, optionally substituted with one, two, three, or four substituents Q^{a}. In certain embodiments, R^{7d} and R^{7e} together with the carbon atoms to which they are attached form 5- or 6-membered heterocyclyl, optionally substituted with one, two, three, or four substituents Q^{a}. In certain embodiments, R^{7d} and R^{7e} together with the carbon atoms to which they are attached form bicyclic heterocyclyl, optionally substituted with one, two, three, or four substituents Q^{a}.

In certain embodiments, m is 0. In certain embodiments, m is 1.

In certain embodiments, n is 0. In certain embodiments, n is 1. In certain embodiments, n is 2. In certain embodiments, n is 3. In certain embodiments, n is 4. In certain embodiments, n is 0, 1, or 2. In certain embodiments, n is 0, 1, 2, or 3. In certain embodiments, n is 1, 2, or 3. In certain embodiments, n is 1 or 2.

In certain embodiments, m is 0, and n is 0, 1, 2, or 3. In certain embodiments, m is 0, n is 0, 1, or 2. In certain embodiments, m is 0, n is 0 or 1. In certain embodiments, m is 0, n is 0. In certain embodiments, m is 0 and n is 1. In certain embodiments, m is 1, n is 0, 1, 2, or 3. In certain embodiments, m is 1, n is 0, 1, or 2. In certain embodiments, m is 1, n is 0 or 1. In certain embodiments, m is 1, n is 0. In certain embodiments, m is 1, n is 1.

In specific embodiments, m is 0, n is 1, and R^{5a} and R^{5b} are each methyl.

In certain embodiments, X is N In certain embodiments, X is CR^{x}, wherein R^{X} is as defined herein. In certain embodiments, X is CH.

In certain embodiments, Y is N In certain embodiments, Y is CR^{x}, wherein R^{X} is as defined herein. In certain embodiments, Y is CH.

In certain embodiments, Z is N In certain embodiments, Z is CR^{x}, wherein R^{X} is as defined herein. In certain embodiments, Z is CH.

In certain embodiments, X, Y, and Z are N. In certain embodiments, X and Y are N, and Z is CH. In certain embodiments, X and Z are N, and Y is CH. In certain embodiments, Y and Z are N, and X is CH.

In certain embodiments, the compound provided herein is not 4-(2-(difluoromethyl)-1H-benzo[d]imidazol-1-yl)-6-morpholino-N-(2-phenyl-2-(pyrrolidin-1-yl)ethyl)-1,3,5-triazin-2-amine. In certain embodiments, the compound provided herein is not 6-(2-(difluoromethyl)-1H-benzo[d]imidazol-1-yl)-N-(1-(4-((R)-3-(methoxymethyl)morpholino)phenyl)ethyl)-2-morpholinopyrimidin-4-amine.

In certain embodiments, when X, Y, and Z are N, and R^{5a} is hydrogen, R^{5b} is not heterocyclyl. In certain embodiments, when X, Y, and Z are N, and R^{5a} is hydrogen, R^{5b} is not 5-membered heterocyclyl. In certain embodiments, when X, Y, and Z are N, and R^{5a} is hydrogen, R^{5b} is not pyrrolidinyl. In certain embodiments, when X, Y, and Z are N, and R^{5a} is hydrogen, R^{5b} is not pyrrolidin-1-yl.

In certain embodiments, when X and Z are N, Y is CH, and R^{5a} is hydrogen, R^{5b} is morpholino-substituted phenyl. In certain embodiments, when X and Z are N, Y is CH, and R^{5a} is hydrogen, R^{5b} is not 4-((R)-3-(methoxymethyl)morpholino)phenyl.

In one embodiment, provided herein is a compound selected from:

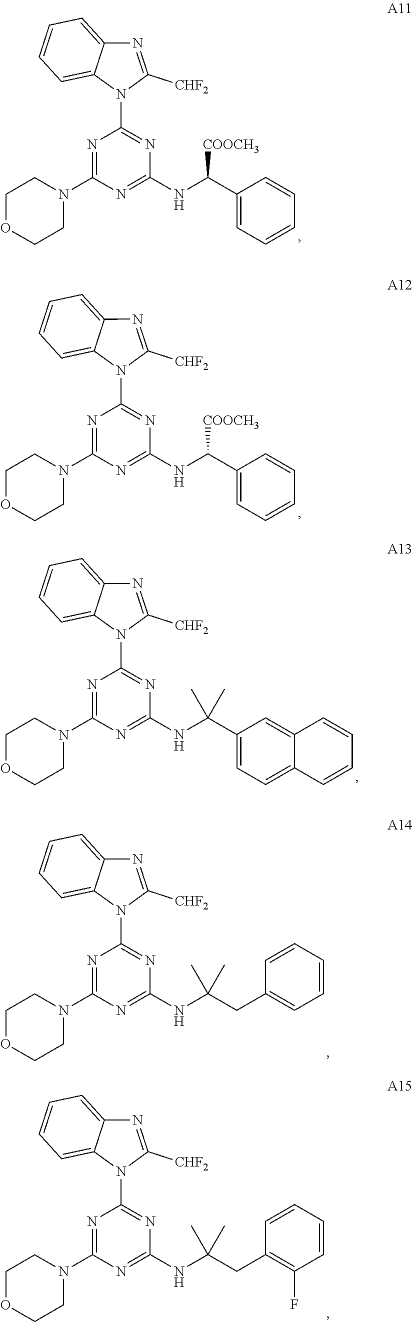

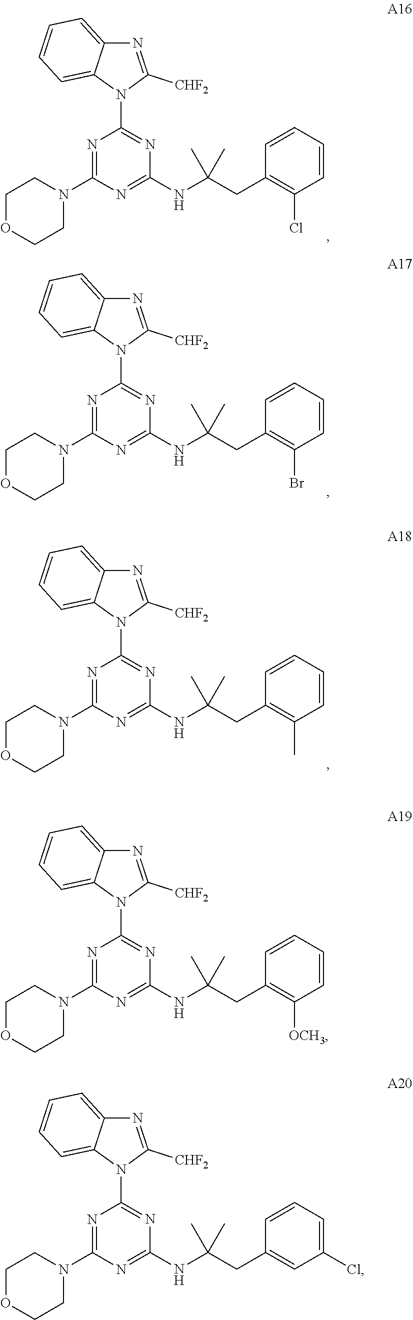

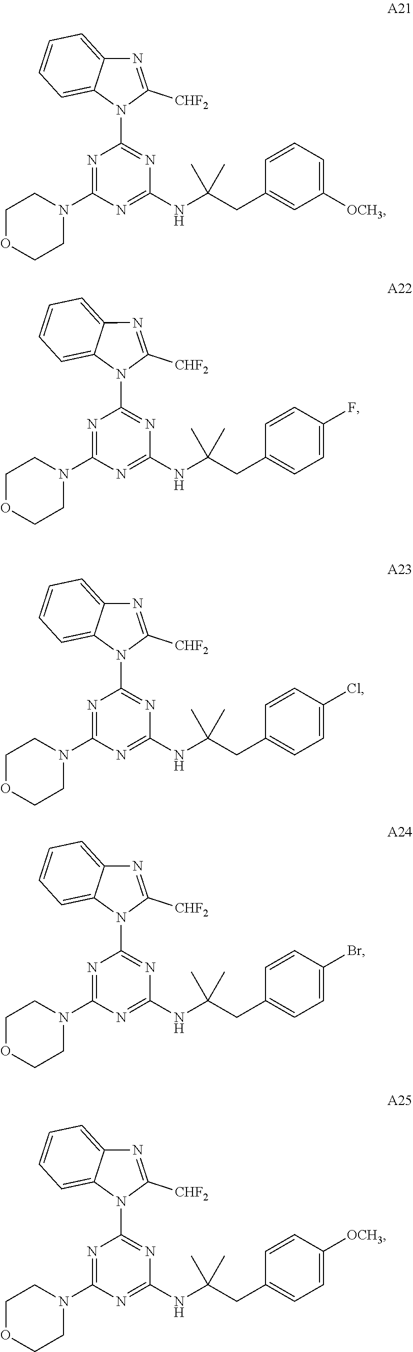

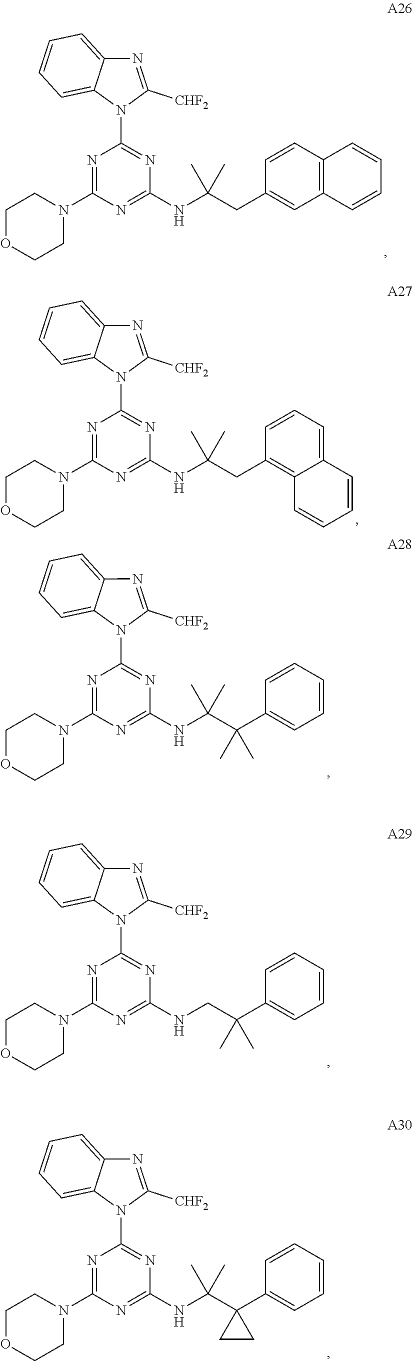

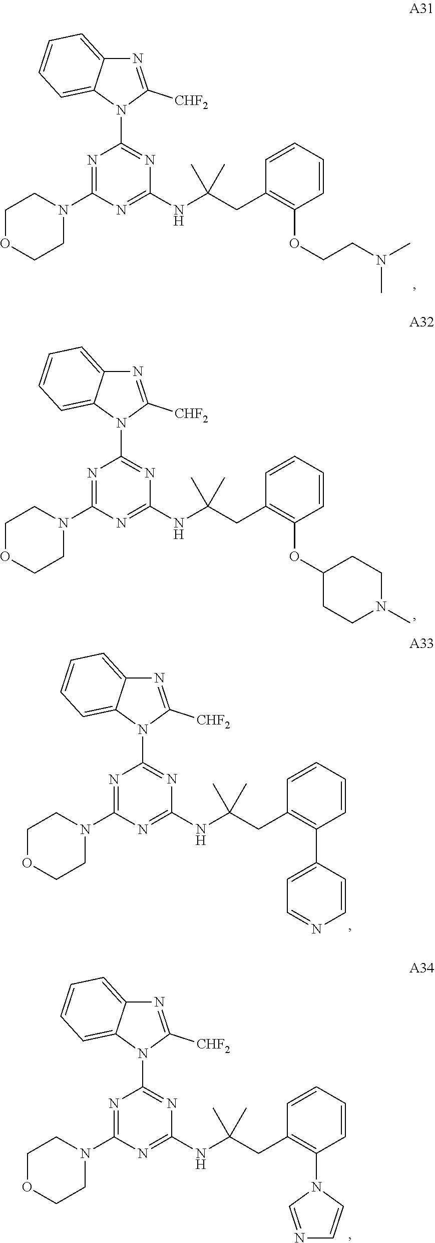 [SMILES: CN(C)CCOc1ccccc1CC(C)(C)Nc1nc(N2CCOCC2)nc(-n2c(C(F)F)nc3ccccc32)n1 ; CN1CCC(Oc2ccccc2CC(C)(C)Nc2nc(N3CCOCC3)nc(-n3c(C(F)F)nc4ccccc43)n2)CC1 ; CC(C)(Cc1ccccc1-c1ccncc1)Nc1nc(N2CCOCC2)nc(-n2c(C(F)F)nc3ccccc32)n1 ; CC(C)(Cc1ccccc1-n1ccnc1)Nc1nc(N2CCOCC2)nc(-n2c(C(F)F)nc3ccccc32)n1]

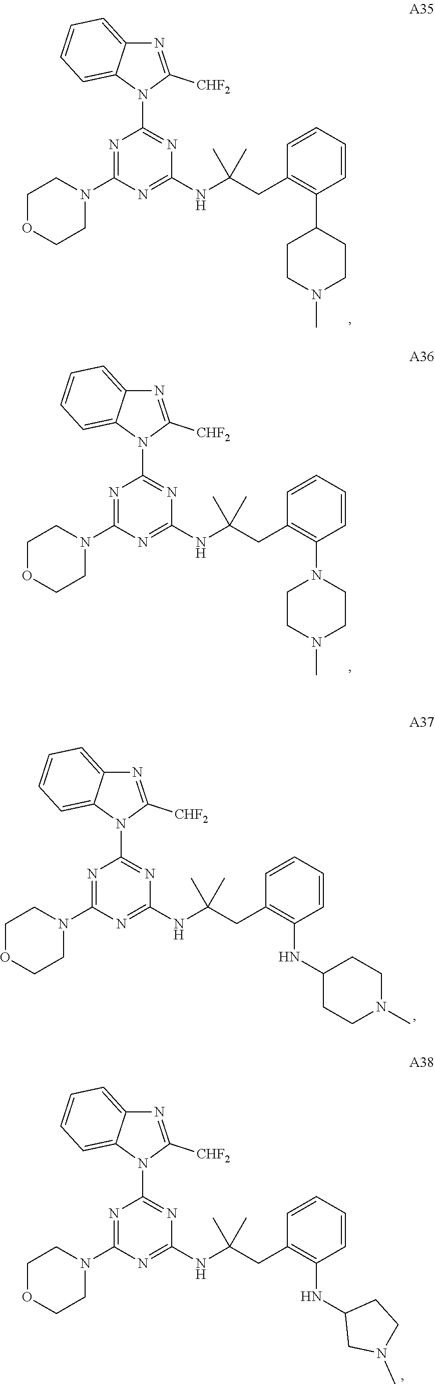 [SMILES: CN1CCC(c2ccccc2CC(C)(C)Nc2nc(N3CCOCC3)nc(-n3c(C(F)F)nc4ccccc43)n2)CC1 ; CN1CCN(c2ccccc2CC(C)(C)Nc2nc(N3CCOCC3)nc(-n3c(C(F)F)nc4ccccc43)n2)CC1 ; CN1CCC(Nc2ccccc2CC(C)(C)Nc2nc(N3CCOCC3)nc(-n3c(C(F)F)nc4ccccc43)n2)CC1 ; CN1CCC(Nc2ccccc2CC(C)(C)Nc2nc(N3CCOCC3)nc(-n3c(C(F)F)nc4ccccc43)n2)C1]

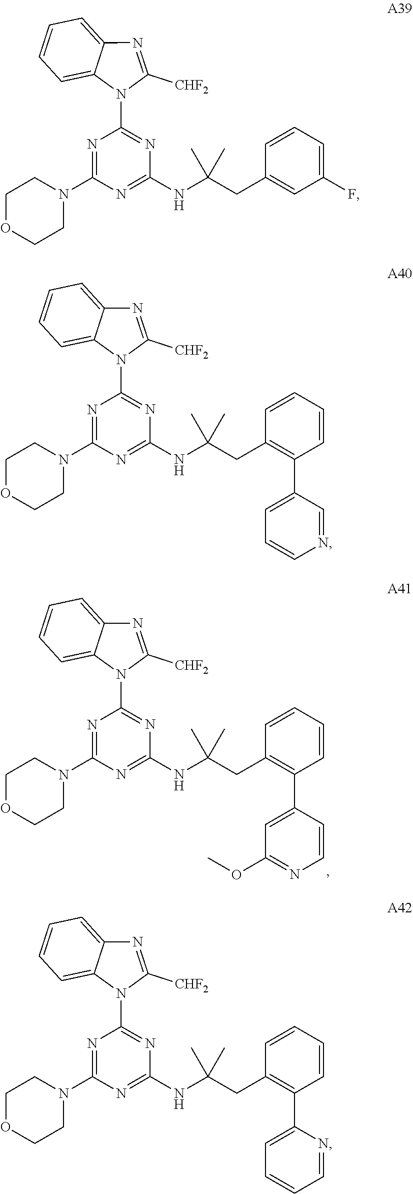 [SMILES: CC(C)(Cc1cccc(F)c1)Nc1nc(N2CCOCC2)nc(-n2c(C(F)F)nc3ccccc32)n1 ; CC(C)(Cc1ccccc1-c1cccnc1)Nc1nc(N2CCOCC2)nc(-n2c(C(F)F)nc3ccccc32)n1 ; COc1cc(-c2ccccc2CC(C)(C)Nc2nc(N3CCOCC3)nc(-n3c(C(F)F)nc4ccccc43)n2)ccn1 ; CC(C)(Cc1ccccc1-c1ccccn1)Nc1nc(N2CCOCC2)nc(-n2c(C(F)F)nc3ccccc32)n1]

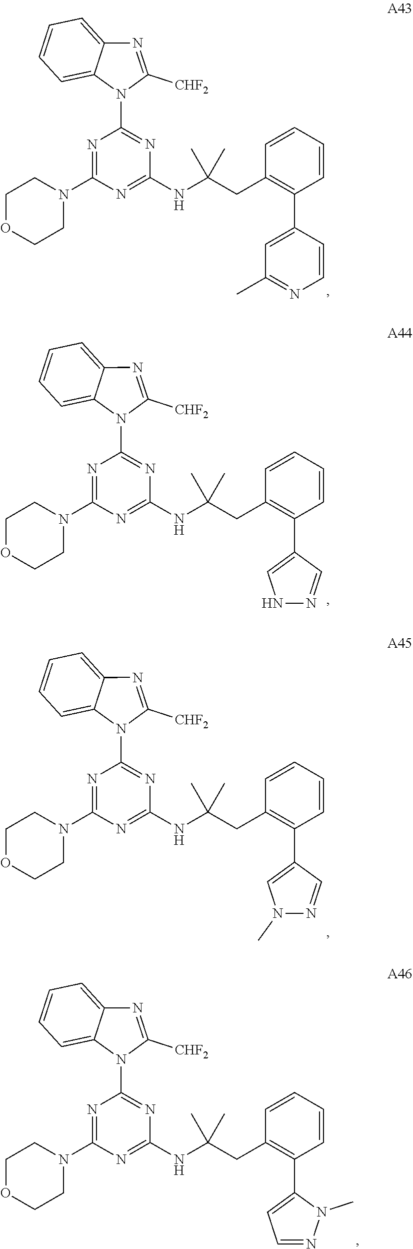 [SMILES: Cc1cc(-c2ccccc2CC(C)(C)Nc2nc(N3CCOCC3)nc(-n3c(C(F)F)nc4ccccc43)n2)ccn1 ; CC(C)(Cc1ccccc1-c1cn[nH]c1)Nc1nc(N2CCOCC2)nc(-n2c(C(F)F)nc3ccccc32)n1 ; Cn1cc(-c2ccccc2CC(C)(C)Nc2nc(N3CCOCC3)nc(-n3c(C(F)F)nc4ccccc43)n2)cn1 ; Cn1nccc1-c1ccccc1CC(C)(C)Nc1nc(N2CCOCC2)nc(-n2c(C(F)F)nc3ccccc32)n1]

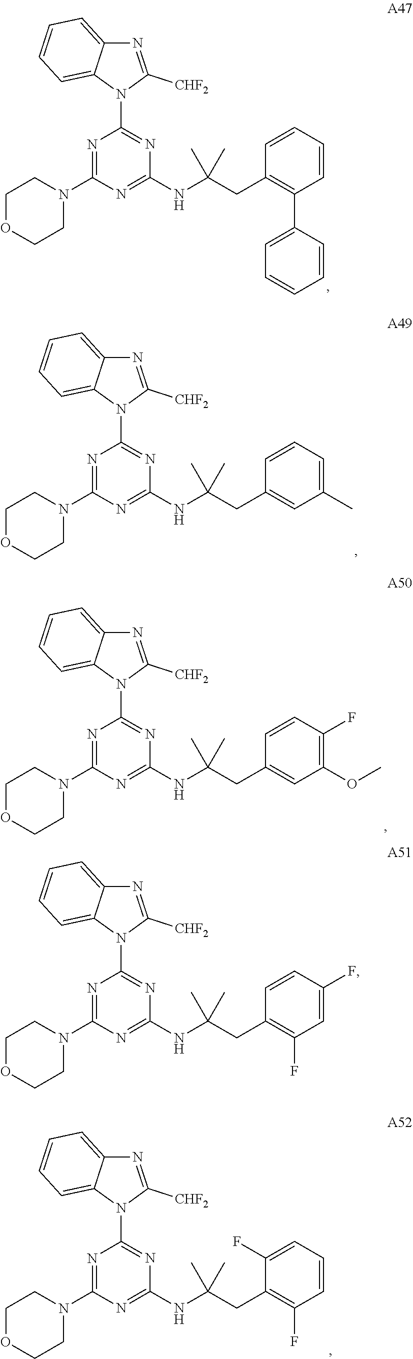

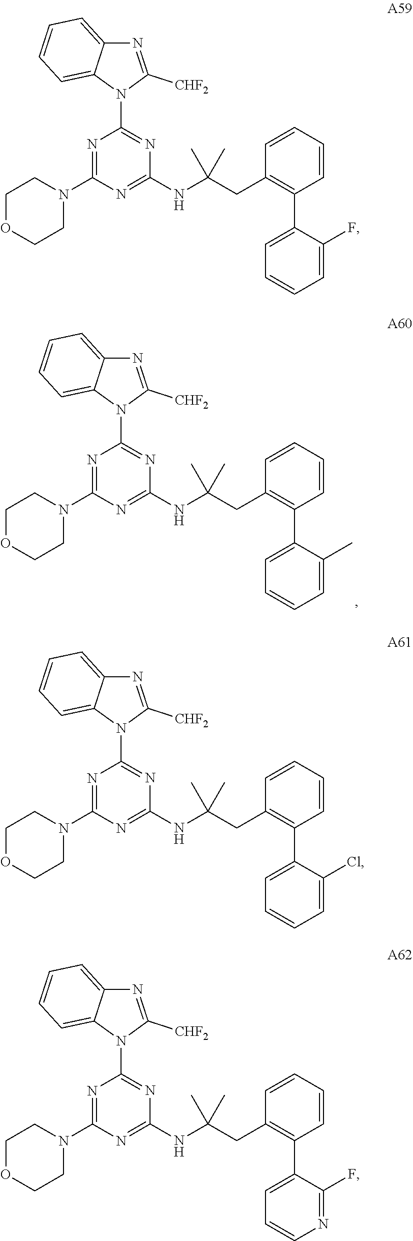 [SMILES: CC(C)(Cc1ccccc1-c1ccccc1F)Nc1nc(N2CCOCC2)nc(-n2c(C(F)F)nc3ccccc32)n1 ; Cc1ccccc1-c1ccccc1CC(C)(C)Nc1nc(N2CCOCC2)nc(-n2c(C(F)F)nc3ccccc32)n1 ; CC(C)(Cc1ccccc1-c1ccccc1Cl)Nc1nc(N2CCOCC2)nc(-n2c(C(F)F)nc3ccccc32)n1 ; CC(C)(Cc1ccccc1-c1cccnc1F)Nc1nc(N2CCOCC2)nc(-n2c(C(F)F)nc3ccccc32)n1]

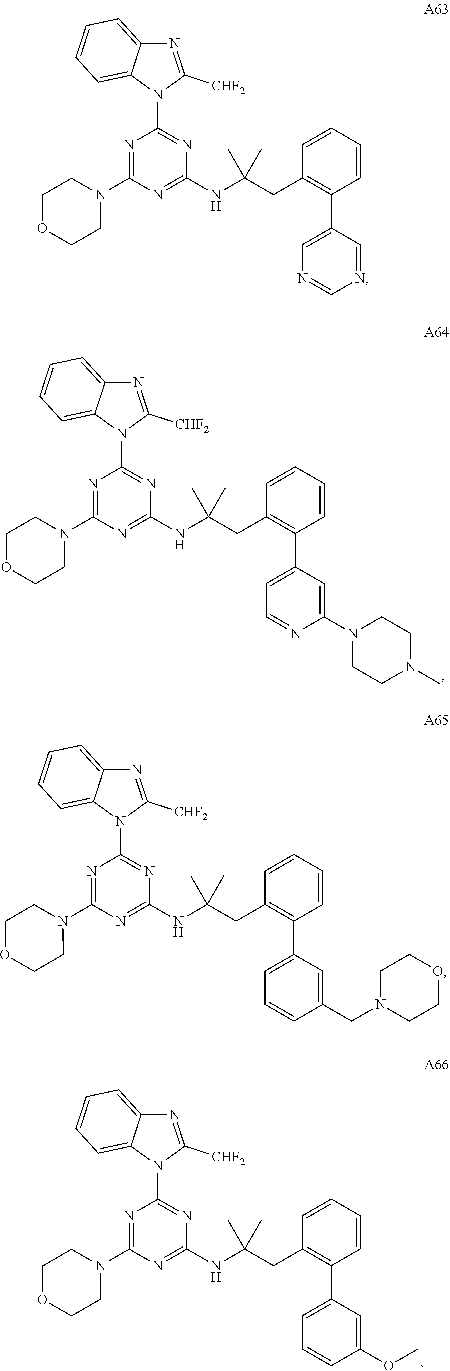 [SMILES: CC(C)(Cc1ccccc1-c1cncnc1)Nc1nc(N2CCOCC2)nc(-n2c(C(F)F)nc3ccccc32)n1 ; CN1CCN(c2cc(-c3ccccc3CC(C)(C)Nc3nc(N4CCOCC4)nc(-n4c(C(F)F)nc5ccccc54)n3)ccn2)CC1 ; CC(C)(Cc1ccccc1-c1cccc(CN2CCOCC2)c1)Nc1nc(N2CCOCC2)nc(-n2c(C(F)F)nc3ccccc32)n1 ; COc1cccc(-c2ccccc2CC(C)(C)Nc2nc(N3CCOCC3)nc(-n3c(C(F)F)nc4ccccc43)n2)c1]

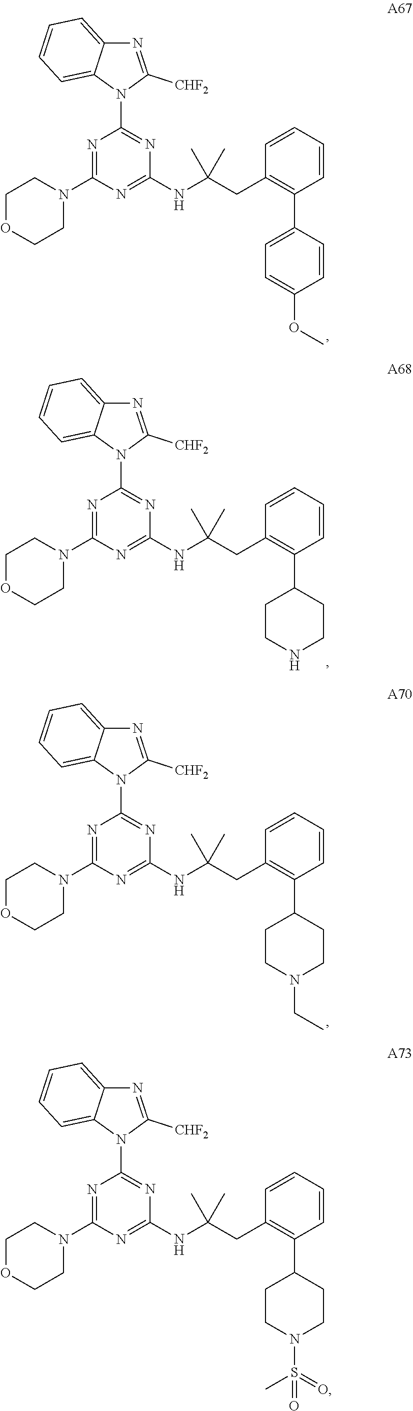 [SMILES: COc1ccc(-c2ccccc2CC(C)(C)Nc2nc(N3CCOCC3)nc(-n3c(C(F)F)nc4ccccc43)n2)cc1 ; CC(C)(Cc1ccccc1C1CCNCC1)Nc1nc(N2CCOCC2)nc(-n2c(C(F)F)nc3ccccc32)n1 ; CCN1CCC(c2ccccc2CC(C)(C)Nc2nc(N3CCOCC3)nc(-n3c(C(F)F)nc4ccccc43)n2)CC1 ; CC(C)(Cc1ccccc1C1CCN(S(C)(=O)=O)CC1)Nc1nc(N2CCOCC2)nc(-n2c(C(F)F)nc3ccccc32)n1]

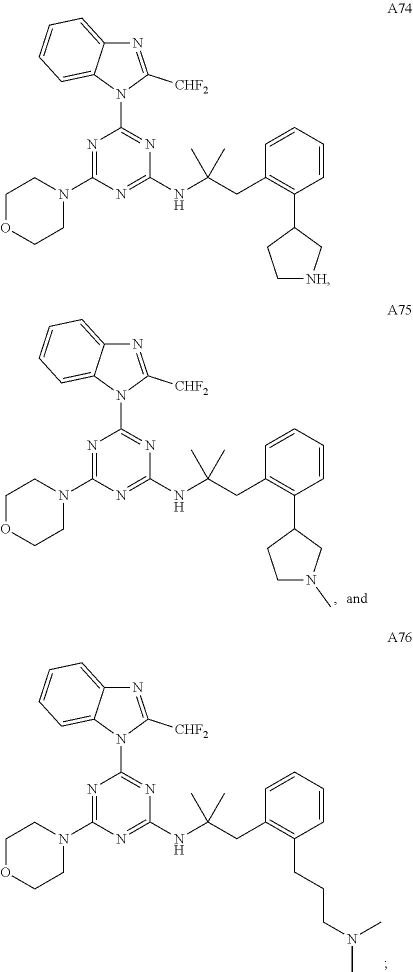 [SMILES: CC(C)(Cc1ccccc1C1CCNC1)Nc1nc(N2CCOCC2)nc(-n2c(C(F)F)nc3ccccc32)n1 ; CN1CCC(c2ccccc2CC(C)(C)Nc2nc(N3CCOCC3)nc(-n3c(C(F)F)nc4ccccc43)n2)C1 ; CN(C)CCCc1ccccc1CC(C)(C)Nc1nc(N2CCOCC2)nc(-n2c(C(F)F)nc3ccccc32)n1]

In one embodiment, the PI3K inhibitor is Compound I, isotopic variants, pharmaceutically acceptable salts, solvates, hydrates, or prodrugs thereof. In one embodiment, the PI3K inhibitor is Compound II, isotopic variants, pharmaceutically acceptable salts, solvates, hydrates, or prodrugs thereof. In one embodiment, the PI3K inhibitor is Compound III, isotopic variants, pharmaceutically acceptable salts, solvates, hydrates, or prodrugs thereof. In one embodiment, the PI3K inhibitor is Compound IV, isotopic variants, pharmaceutically acceptable salts, solvates, hydrates, or prodrugs thereof. In one embodiment, the PI3K inhibitor is Compound V, isotopic variants, pharmaceutically acceptable salts, solvates, hydrates, or prodrugs thereof. In one embodiment, the PI3K inhibitor is Compound VI, isotopic variants, pharmaceutically acceptable salts, solvates, hydrates, or prodrugs thereof. In one embodiment, the PI3K inhibitor is Compound VII, isotopic variants, pharmaceutically acceptable salts, solvates, hydrates, or prodrugs thereof. In one embodiment, the PI3K inhibitor is Compound VIII, isotopic variants, pharmaceutically acceptable salts, solvates, hydrates, or prodrugs thereof. In one embodiment, the PI3K inhibitor is Compound IX, isotopic variants, pharmaceutically acceptable salts, solvates, hydrates, or prodrugs thereof. In one embodiment, the PI3K inhibitor is Compound X, isotopic variants, pharmaceutically acceptable salts, solvates, hydrates, or prodrugs thereof. In one embodiment, the PI3K inhibitor is Compound XI, isotopic variants, pharmaceutically acceptable salts, solvates, hydrates, or prodrugs thereof. In one embodiment, the PI3K inhibitor is Compound XII, isotopic variants, pharmaceutically acceptable salts, solvates, hydrates, or prodrugs thereof. In one embodiment, the PI3K inhibitor is Compound XIII, isotopic variants, pharmaceutically acceptable salts, solvates, hydrates, or prodrugs thereof. In one embodiment, the PI3K inhibitor is Compound XIV, isotopic variants, pharmaceutically acceptable salts, solvates, hydrates, or prodrugs thereof. In one embodiment, the PI3K inhibitor is Compound XV, isotopic variants, pharmaceutically acceptable salts, solvates, hydrates, or prodrugs thereof. In one embodiment, the PI3K inhibitor is Compound XVI, isotopic variants, pharmaceutically acceptable salts, solvates, hydrates, or prodrugs thereof.

### Second Agents

Some embodiments provided herein describe pharmaceutical compositions or methods for using the pharmaceutical compositions comprising a PI3K inhibitor described herein in combination with a BTK inhibitor.

Any suitable BTK inhibitor may be used in combination with a PI3K inhibitor described herein. In some embodiments, the BTK inhibitor is ibrutinib, BGB-3111, CC-292, ACP 196, CNX-774, CGI1746, LFM-A13, CNX-774, ONO-4059, RN486 CPI-0610, DUAL946, GSK525762, I-BET151, JQ1, OTX015, PFI-1, RVX-208, RVX2135, TEN-010, or pharmaceutically acceptable salts thereof. In some embodiments, the BTK inhibitor is ibrutinib, or a pharmaceutically acceptable salt thereof, or BGB-3111, or a pharmaceutically acceptable salt thereof. In another embodiment, the BTK inhibitor is ibrutinib or a pharmaceutically acceptable salt thereof. In yet another embodiment, the BTK inhibitor is BGB-3111 or a pharmaceutically acceptable salt thereof.

### Methods of Use

In certain embodiments, provided herein are methods for treating or preventing a disease comprising administering an effective amount of a compound of Formula (I), or an isotopic variant thereof or a pharmaceutically acceptable salt, solvate, hydrate, or prodrug thereof and an effective amount of a BTK inhibitor. In some embodiments, the BTK inhibitor is ibrutinib or BGB-3111, or pharmaceutically acceptable salts thereof. In some embodiments, the BTK inhibitor is BGB-3111, or pharmaceutically acceptable salts thereof. In some embodiments, the compound of Formula (I) is Compound I or an isotopic variant, pharmaceutically acceptable salt, solvate, hydrate, or prodrug thereof. In some embodiments, the compound of Formula (I) is Compound II or an isotopic variant, pharmaceutically acceptable salt, solvate, hydrate, or prodrug thereof. In some embodiments, the compound of Formula (I) is Compound III or an isotopic variant, pharmaceutically acceptable salt, solvate, hydrate, or prodrug thereof. In some embodiments, the compound of Formula (I) is Compound IV or an isotopic variant, pharmaceutically acceptable salt, solvate, hydrate, or prodrug thereof. In some embodiments, the compound of Formula (I) is Compound V or an isotopic variant, pharmaceutically acceptable salt, solvate, hydrate, or prodrug thereof. In some embodiments, the compound of Formula (I) is Compound VI or an isotopic variant, pharmaceutically acceptable salt, solvate, hydrate, or prodrug thereof. In some embodiments, the compound of Formula (I) is Compound VII or an isotopic variant, pharmaceutically acceptable salt, solvate, hydrate, or prodrug thereof. In some embodiments, the compound of Formula (I) is Compound VIII or an isotopic variant, pharmaceutically acceptable salt, solvate, hydrate, or prodrug thereof. In some embodiments, the compound of Formula (I) is Compound IX or an isotopic variant, pharmaceutically acceptable salt, solvate, hydrate, or prodrug thereof. In some embodiments, the compound of Formula (I) is Compound X or an isotopic variant, pharmaceutically acceptable salt, solvate, hydrate, or prodrug thereof. In some embodiments, the compound of Formula (I) is Compound XI or an isotopic variant, pharmaceutically acceptable salt, solvate, hydrate, or prodrug thereof. In some embodiments, the compound of Formula (I) is Compound XII or an isotopic variant, pharmaceutically acceptable salt, solvate, hydrate, or prodrug thereof. In some embodiments, the compound of Formula (I) is Compound XIII or an isotopic variant, pharmaceutically acceptable salt, solvate, hydrate, or prodrug thereof. In some embodiments, the compound of Formula (I) is Compound XIV or an isotopic variant, pharmaceutically acceptable salt, solvate, hydrate, or prodrug thereof. In some embodiments, the compound of Formula (I) is Compound XV or an isotopic variant, pharmaceutically acceptable salt, solvate, hydrate, or prodrug thereof. In some embodiments, the compound of Formula (I) is Compound XVI or an isotopic variant, pharmaceutically acceptable salt, solvate, hydrate, or prodrug thereof.

In one embodiment, provided herein are methods for treating or preventing cancer, comprising administering a compound of Formula (I), or an isotopic variant thereof; or a pharmaceutically acceptable salt, solvate, hydrate, or prodrug thereof and an effective amount of a BTK inhibitor to a subject in need thereof. In one embodiment, the BTK inhibitor is ibrutinib or BGB-3111, or pharmaceutically acceptable salts thereof. In some embodiments, the compound of Formula (I) is Compound I. In some embodiments, the compound of Formula (I) is Compound II. In some embodiments, the compound of Formula (I) is Compound III. In some embodiments, the compound of Formula (I) is Compound IV. In some embodiments, the compound of Formula (I) is Compound V. In some embodiments, the compound of Formula (I) is Compound VI. In some embodiments, the compound of Formula (I) is Compound VII. In some embodiments, the compound of Formula (I) is Compound VIII. In some embodiments, the compound of Formula (I) is Compound IX. In some embodiments, the compound of Formula (I) is Compound X. In some embodiments, the compound of Formula (I) is Compound XI. In some embodiments, the compound of Formula (I) is Compound XII. In some embodiments, the compound of Formula (I) is Compound XIII. In some embodiments, the compound of Formula (I) is Compound XIV. In some embodiments, the compound of Formula (I) is Compound XV. In some embodiments, the compound of Formula (I) is Compound XVI.

In certain embodiments, the proliferative disease is cancer. In certain embodiments, the proliferative disease is a hematological cancer or malignancy.

In certain embodiments, the proliferative disease is a cancer of the breast, skin, prostate, cervix, uterus, ovary, testes, bladder, lung, liver, larynx, oral cavity, colon and gastrointestinal tract (e.g., esophagus, stomach, pancreas), brain, thyroid, blood, and lymphatic system.

In certain embodiments, the cancer treatable with the methods provided herein includes, but is not limited to, (1) leukemias, including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome or a symptom thereof (such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia, and chronic myelomonocytic leukemia (CMML), (2) chronic leukemias, including, but not limited to, chronic myelocytic (granulocytic) leukemia, chronic lymphocytic leukemia, and hairy cell leukemia; (3) polycythemia vera; (4) lymphomas, including, but not limited to, Hodgkin's disease and non-Hodgkin's disease; (5) multiple myelomas, including, but not limited to, smoldering multiple myeloma, nonsecretory myeloma, osteosclerotic myeloma, plasma cell leukemia, solitary plasmacytoma, and extramedullary plasmacytoma; (6) Waldenstrom's macroglobulinernia; (7) monoclonal gammopathy of undetermined significance; (8) benign monoclonal gammopathy; (9) heavy chain disease; (10) bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, and synovial sarcoma; (11) brain tumors, including, but not limited to, glioma, astrocytoma, brain stem glioma, ependymoma, aligodendrogliorna, nonglial tumor, acoustic neurinoma, craniopharyngioma, medulloblastoma, meningioma, pineocytoma, pineoblastoma, and primary brain lymphoma; (12) breast cancer, including, but not limited to, adenocarcinoma, lobular (small cell) carcinoma, intraductal carcinoma, medullary breast cancer, mutinous breast cancer, tubular breast cancer, papillary breast cancer, primary cancers, Paget's disease, and inflammatory breast cancer; (13) adrenal cancer, including, but not limited to, pheochromocytom and adrenocortical carcinoma; (14) thyroid cancer, including, but not limited to, papillary or follicular thyroid cancer, medullary thyroid cancer, and anaplastic thyroid cancer; (15) pancreatic cancer, including, but not limited to, insulinoma, gastrinoma, glucagonoma, vipoma, somatostatin-secreting tumor, and carcinoid or islet cell tumor; (16) pituitary cancer, including, but limited to, Cushing's disease, prol actin-secreting tumor, acromegaly, and diabetes insipius; (17) eye cancer, including, but not limited, to ocular melanoma such as iris melanoma, choroidal melanoma, and cilliary body melanoma, and retinoblastoma; (18) vaginal cancer, including, but not limited to, squamous cell carcinoma, adenocarcinoma, and melanoma; (19) vulvar cancer, including, but not limited to, squamous cell carcinoma, melanoma, adenocarcinoma, basal cell carcinoma, sarcoma, and Paget's disease; (20) cervical cancers, including, but not limited to, squamous cell carcinoma, and adenocarcinoma; (21) uterine cancer, including, but not limited to, endometrial carcinoma and uterine sarcoma; (22) ovarian cancer, including, but not limited to, ovarian epithelial carcinoma, borderline tumor, germ cell tumor, and stromal tumor; (23) esophageal cancer, including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; (24) stomach cancer, including, but not limited to, adenocarcinoma, fungating (polypoid), ulcerating, superficial spreading, diffusely spreading, malignant lymphoma, liposarcoma, fibrosarcoma, and carcinosarcoma; (25) colon cancer; (26) rectal cancer; (27) liver cancer, including, but not limited to, hepatocellular carcinoma and hepatoblastoma; (28) gallbladder cancer, including, but not limited to, adenocarcinoma; (29) cholangiocarcinomas, including, but not limited to, pappillary, nodular, and diffuse; (30) lung cancer, including, but not limited to, non-small cell lung cancer, squamous cell carcinoma (epidermoid carcinoma), adenocarcinoma, large-cell carcinoma, and small-cell lung cancer; (31) testicular cancer, including, but not limited to, germinal tumor, seminoma, anaplastic, classic (typical), spermatocytic, nonserninoma, embryonal carcinoma, teratoma carcinoma, and choriocarcinoma (yolk-sac tumor); (32) prostate cancer, including, but not limited to, adenocarcinoma, leiomyosarcoma, and rhabdomyosarcoma; (33) penal cancer; (34) oral cancer, including, but not limited to, squamous cell carcinoma; (35) basal cancer; (36) salivary gland cancer, including, but not limited to, adenocarcinoma, mucoepidermoid carcinoma, and adenoidcystic carcinoma; (37) pharynx cancer, including, but not limited to, squamous cell cancer and verrucous; (38) skin cancer, including, but not limited to, basal cell carcinoma, squamous cell carcinoma and melanoma, superficial spreading melanoma, nodular melanoma, lentigo malignant melanoma, and acral lentiginous melanoma; (39) kidney cancer, including, but not limited to, renal cell cancer, adenocarcinoma, hypernephroma, fibrosarcoma, and transitional cell cancer (renal pelvis and/or uterer); (40) Wilms' tumor; (41) bladder cancer, including, but not limited to, transitional cell carcinoma, squamous cell cancer, adenocarcinoma, and carcinosarcoma; and other cancer, including, not limited to, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas (See Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In certain embodiments, provided herein are methods of treating a hematological malignancy with a combination of an effective amount of a compound of Formula (I), or an isotopic variant thereof; or a pharmaceutically acceptable salt, solvate, hydrate, or prodrug thereof and an effective amount of a BTK inhibitor (e.g., BGB-3111) in a patient. In certain embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is chronic lymphocytic leukemia, follicular lymphoma, diffuse large B-cell lymphoma, or non-Hodgkin's lymphoma. In some embodiments, the hematological malignancy is chronic lymphocytic leukemia or non-Hodgkin's lymphoma. In some embodiments, the hematological malignancy is chronic lymphocytic leukemia. In other embodiments, the hematological malignancy is non-Hodgkin's lymphoma. In some embodiments, the hematological malignancy is follicular lymphoma. In other embodiments, the hematological malignancy is diffuse large B-cell lymphoma. In some embodiments, the compound of Formula (I) is Compound I or an isotopic variant, pharmaceutically acceptable salt, solvate, hydrate, or prodrug thereof. In some embodiments, the compound of Formula (I) is Compound II or an isotopic variant, pharmaceutically acceptable salt, solvate, hydrate, or prodrug thereof. In some embodiments, the compound of Formula (I) is Compound III or an isotopic variant, pharmaceutically acceptable salt, solvate, hydrate, or prodrug thereof. In some embodiments, the compound of Formula (I) is Compound IV or an isotopic variant, pharmaceutically acceptable salt, solvate, hydrate, or prodrug thereof. In some embodiments, the compound of Formula (I) is Compound V or an isotopic variant, pharmaceutically acceptable salt, solvate, hydrate, or prodrug thereof. In some embodiments, the compound of Formula (I) is Compound VI or an isotopic variant, pharmaceutically acceptable salt, solvate, hydrate, or prodrug thereof. In some embodiments, the compound of Formula (I) is Compound VII or an isotopic variant, pharmaceutically acceptable salt, solvate, hydrate, or prodrug thereof. In some embodiments, the compound of Formula (I) is Compound VIII or an isotopic variant, pharmaceutically acceptable salt, solvate, hydrate, or prodrug thereof. In some embodiments, the compound of Formula (I) is Compound IX or an isotopic variant, pharmaceutically acceptable salt, solvate, hydrate, or prodrug thereof. In some embodiments, the compound of Formula (I) is Compound X or an isotopic variant, pharmaceutically acceptable salt, solvate, hydrate, or prodrug thereof. In some embodiments, the compound of Formula (I) is Compound XI or an isotopic variant, pharmaceutically acceptable salt, solvate, hydrate, or prodrug thereof. In some embodiments, the compound of Formula (I) is Compound XII or an isotopic variant, pharmaceutically acceptable salt, solvate, hydrate, or prodrug thereof. In some embodiments, the compound of Formula (I) is Compound XIII or an isotopic variant, pharmaceutically acceptable salt, solvate, hydrate, or prodrug thereof. In some embodiments, the compound of Formula (I) is Compound XIV or an isotopic variant, pharmaceutically acceptable salt, solvate, hydrate, or prodrug thereof. In some embodiments, the compound of Formula (I) is Compound XV or an isotopic variant, pharmaceutically acceptable salt, solvate, hydrate, or prodrug thereof. In some embodiments, the compound of Formula (I) is Compound XVI or an isotopic variant, pharmaceutically acceptable salt, solvate, hydrate, or prodrug thereof.

In certain embodiments, the hematological malignancy is a T-cell malignancy. In certain embodiments, T-cell malignancies include peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In certain embodiments, the hematological malignancy is a B-cell malignancy. In certain embodiments, B-cell malignancies include acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In certain embodiments, the B-cell malignancy is diffuse large B-cell lymphoma (DLBCL). In certain embodiments, the hematological malignancy is diffuse large B-cell lymphoma (DLBCL). In certain embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In certain embodiments, the hematological malignancy is relapsed-refractory diffuse large B-cell lymphoma (r/r DLBCL).

In some embodiments, the hematological malignancy is B-cell non-Hodgkin's lymphoma (NHL). In some embodiments, the hematological malignancy is B-cell indolent non-Hodgkin's lymphoma (NHL). In certain embodiments, the B-cell malignancy is selected from chronic lymphocytic leukemia (CLL), small lymphocytic lymphoma (SLL), follicular lymphoma (FL), marginal zone B cell lymphoma (MZL), diffuse large B-cell lymphoma (DLBCL), and high grade non-Hodgkin's lymphoma. In certain embodiments, the B-cell malignancy is selected from chronic lymphocytic leukemia (CLL), follicular lymphoma (FL), marginal zone B cell lymphoma (MZL), or diffuse large B-cell lymphoma (DLBCL).

In certain embodiments, the hematological malignancy is a relapsed or refractory hematological malignancy. In certain embodiments, the relapsed or refractory hematological malignancy is a relapsed or refractory T-cell malignancy. In certain embodiments, the relapsed or refractory hematological malignancy is a relapsed or refractory B-cell malignancy. In some embodiments, the cancer is relapsed B-cell non-Hodgkin's lymphoma (NHL) or chronic lymphocytic leukemia (CLL). In some embodiments, the hematological malignancy is relapsed B-cell non-Hodgkin's lymphoma (NHL) or chronic lymphocytic leukemia (CLL).

Some embodiments provided herein describe a method for treating or preventing a proliferative disease or disorder comprising administering a PI3K inhibitor in combination with a BTK inhibitor. In some embodiments provided herein is a method for preventing relapse of a proliferative disease or disorder, the method comprising administering a PI3K inhibitor in combination with a BTK inhibitor. In some embodiments provided herein is a method for achieving and retaining partial cancer remission, the method comprising administering a PI3K inhibitor in combination with a BTK inhibitor. In some embodiments provided herein is a method for achieving and retaining complete cancer remission, the method comprising administering a PI3K inhibitor in combination with a BTK inhibitor. In some embodiments, the combination therapy of a PI3K inhibitor described herein (e.g., a compound of Formula (I)) and a BTK inhibitor (e.g., BGB-3111) provides a synergistic effect. In some embodiments, the combination therapy of a PI3K inhibitor described herein (e.g., a compound of Formula (I)) and a BTK inhibitor (e.g., BGB-3111) provides a synergistic antitumor or anti-cancer activity. In certain embodiments, the combination therapy described herein permits the use of lower dosages of the PI3K inhibitor and/or the BTK inhibitor (e.g., BGB-3111). In some embodiments, the combination therapy described herein permits less frequent administration of the PI3K inhibitor and/or the BTK inhibitor (e.g., BGB-3111) to a subject. In some embodiments, the combination therapy described herein reduces the toxicity associated with the administration of the PI3K inhibitor and/or the BTK inhibitor (e.g., BGB-3111) to a subject without reducing the efficacy in the prevention, management, treatment, or amelioration of cancer, such as chronic lymphocytic leukemia. In some embodiments, the synergistic effect observed with the combination therapy described herein results in improved efficacy of therapies in the prevention, management, treatment, or amelioration of cancer, such as chronic lymphocytic leukemia.

In some embodiments, the combination therapy described herein avoids or reduces adverse or unwanted side effects associated with the use of the PI3K inhibitor and/or the BTK inhibitor (e.g., BGB-3111).

In some embodiments, the combination therapy described herein avoids or reduces adverse or unwanted side effects associated with the use of the PI3K inhibitor and/or the BTK inhibitor. In some embodiments, the combination therapy described herein avoids, reduces, or minimizes the risk of death due to infections. In some embodiments, the combination therapy described herein avoids, reduces, or minimizes infections, neutropenia, diarrhea/colitis, elevated liver transaminases (alanine aminotransferase/aspartate aminotransferase >5× upper limit of normal), pneumonitis, rash, hepatic impairment, renal impairment, pyrexia, or increased triglycerides, or a combination thereof in patients receiving the combination therapy. In certain embodiments, the combination therapy described herein avoids, reduces, or minimizes the incidence of infection associated with the use of the PI3K inhibitor and/or the BTK0 inhibitor. In certain embodiments, the combination therapy described herein avoids, reduces, or minimizes the incidence of neutropenia. In certain embodiments, the combination therapy described herein avoids, reduces, or minimizes the incidence of diarrhea/colitis. In certain embodiments, the combination therapy described herein avoids, reduces, or minimizes the incidence of elevated liver transaminases. In certain embodiments, the combination therapy described herein avoids, reduces, or minimizes the incidence of pneumonitis. In certain embodiments, the combination therapy described herein avoids, reduces, or minimizes the incidence of a rash. In certain embodiments, the combination therapy described herein avoids, reduces, or minimizes the incidence of hepatic impairment or renal impairment. In certain embodiments, the combination therapy described herein avoids, reduces, or minimizes the incidence of pyrexia. In certain embodiments, the combination therapy described herein avoids, reduces, or minimizes the incidence of increased triglycerides. In certain embodiments, the combination therapy described herein avoids, reduces, or minimizes enterocolitis (manifested as diarrhea), cutaneous toxicities, liver toxicity (manifested as elevation of transaminases), pulmonary toxicity (manifested as non-infectious pneumonitis), infections, or combinations thereof.

In some embodiments, the combination therapy described herein provides a high objective response rate (ORR) as determined by tumor assessment from radiological tests and/or physical examination. In some embodiments, the combination therapy described herein provides a durable response (DR) and/or increased durable response rate (DRR; a continuous response [complete or partial objective response] beginning within 12 months of treatment and lasting ≥6 months) in the subject or patient. In some embodiments, the combination therapy described herein provides complete remission. In some embodiments, the combination therapy described herein provides a better response compared to the monotherapy treatment of a compound of formula (I) and/or a BTK0 inhibitor. In some embodiments, the combination therapy described herein provides complete remission beginning within 12 months of treatment and lasting ≥6 months. In some embodiments, the combination therapy described herein provides a complete response (CR) and/or no evidence of disease (NED) beginning within 12 months of treatment and lasting ≥6 months.

In some embodiments, the combination therapy described herein avoids, reduces, or minimizes infections, neutropenia, diarrhea, pneumonia, anemia, thrombocytopenia, nausea, vomiting, swelling in extremities, or a combination thereof in patients receiving the combination therapy. In certain embodiments, the combination therapy described herein avoids, reduces, or minimizes the incidence of infection. In certain embodiments, the combination therapy described herein avoids, reduces, or minimizes the incidence of neutropenia. In certain embodiments, the combination therapy described herein avoids, reduces, or minimizes the incidence of diarrhea/colitis. In certain embodiments, the combination therapy described herein avoids, reduces, or minimizes the incidence of pneumonia or pneumonitis. In certain embodiments, the combination therapy described herein avoids, reduces, or minimizes the incidence of anemia. In certain embodiments, the combination therapy described herein avoids, reduces, or minimizes the incidence of thrombocytopenia. In certain embodiments, the combination therapy described herein avoids, reduces, or minimizes the incidence of nausea. In certain embodiments, the combination therapy described herein avoids, reduces, or minimizes the incidence of vomiting. In certain embodiments, the combination therapy described herein avoids, reduces, or minimizes the incidence of swelling in the extremities.

Resistant, relapsed or refractory refers to when a cancer that has a reduced responsiveness to a treatment, e.g., up to the point where the cancer does not respond to treatment. The cancer can be resistant at the beginning of treatment, or it may become resistant during treatment. The term “refractory” can refer to a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease).

Depending on the disorder, disease, or condition to be treated, and the subject's condition, the compounds or pharmaceutical compositions provided herein can be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration and can be formulated, alone or together, in suitable dosage unit with pharmaceutically acceptable excipients, carriers, adjuvants, and vehicles appropriate for each route of administration as described elsewhere herein.

### Dosages and Dosing Regimens

In certain embodiments, the methods provided herein comprise administering a compound of Formula (I), or an isotopic variant thereof; or a pharmaceutically acceptable salt, solvate, hydrate, or prodrug thereof and a BTK inhibitor (e.g. BGB-3111 or zanubrutinib), to a patient simultaneously or sequentially by the same or different routes of administration.

The suitability of a particular route of administration employed for a particular active agent will depend on the active agent itself (e.g., whether it can be administered orally without decomposing prior to entering the blood stream) and the disease being treated.

In certain embodiments, the compound of Formula (I), or an isotopic variant thereof; or a pharmaceutically acceptable salt, solvate, hydrate, or prodrug thereof and a BTK inhibitor is administered simultaneously, at essentially the same time, or sequentially. If administration takes place sequentially, the BTK inhibitor may be administered before or after administration of a compound of Formula (I), or an isotopic variant thereof; or a pharmaceutically acceptable salt, solvate, hydrate, or prodrug thereof. In some embodiments, the BTK inhibitor is administered before administration of a compound of Formula (I), or an isotopic variant thereof; or a pharmaceutically acceptable salt, solvate, hydrate, or prodrug thereof. In some embodiments, the BTK inhibitor is administered simultaneously with administration of a compound of Formula (I), an isotopic variant thereof; or a pharmaceutically acceptable salt, solvate, hydrate, or prodrug thereof. In some embodiments, the BTK inhibitor is administered after the administration of a compound of Formula (I), an isotopic variant thereof; or a pharmaceutically acceptable salt, solvate, hydrate, or prodrug thereof.

A compound of Formula (I), or an isotopic variant thereof; or a pharmaceutically acceptable salt, solvate, hydrate, or prodrug thereof and the BTK inhibitor need not be administered by means of the same vehicle. In some embodiments, the BTK inhibitor and a compound of Formula (I), or an isotopic variant thereof; or a pharmaceutically acceptable salt, solvate, hydrate, or prodrug thereof are administered in different vehicles. The BTK inhibitor may be administered one or more times, and the number of administrations of each component of the combination may be the same or different. In addition, a compound of Formula (I), or an isotopic variant thereof, or a pharmaceutically acceptable salt, solvate, hydrate, or prodrug thereof and the BTK inhibitor need not be administered at the same site.

In some instances, the methods described herein further comprise administering the PI3K inhibitor in combination with BTK inhibitor to the subject or patient in need thereof in multiple cycles repeated on a regular schedule with periods of rest in between each cycle. For example, in some instances, treatment is given for one week followed by three weeks of rest is one treatment cycle.

In some instances, a cycle comprises administration of the PI3K inhibitor at the same time as administration of the BTK inhibitor. In some instances, the PI3K inhibitor and the BTK inhibitor are administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days.

In some instances, a cycle comprises administration of the PI3K inhibitor first followed by administration of the BTK inhibitor second. In some instances, the PI3K inhibitor is administered for about 1 day, about 2 days, about 3 days, about 4 days, about 5 days, about 6 days, about 7 days, about 8 days, about 9 days, about 10 days, about 11 days, about 12 days, about 13 days, or about 14 days followed by administration of the BTK inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days.

In some instances, a cycle comprises administration of the PI3K inhibitor first followed by concurrent administration of the BTK inhibitor. In some instances, the PI3K inhibitor is first administered for about 1 day, about 2 days, about 3 days, about 4 days, about 5 days, about 6 days, about 7 days, about 8 days, about 9 days, about 10 days, about 11 days, about 12 days, about 13 days, or about 14 days followed by the concurrent administration of the BTK inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days. In some instances, the PI3K inhibitor is first administered for about 1 day, about 2 days, about 3 days, about 4 days, about 5 days, about 6 days, or about 7 days followed by the concurrent administration of the BTK inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days. In some instances, the PI3K inhibitor is first administered for about 7 days followed by the concurrent administration of the BTK inhibitor for about 1 day, about 2 days, about 3 days, about 4 days, about 5 days, about 6 days, about 7 days, about 8 days, about 9 days, about 10 days, about 11 days, about 12 days, about 13 days, or about 14 days. In some instances, the PI3K inhibitor is first administered for about 7 days followed by the concurrent administration of the BTK inhibitor for about 10 days, about 11 days, about 12 days, about 13 days, or about 14 days.

In some instances, a cycle comprises administration of the PI3K inhibitor only. In some instances, the PI3K inhibitor is administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days.

In some instances, a cycle comprises administration of the BTK inhibitor only. In some instances, the BTK inhibitor is administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days.

In some instances, the method for multiple cycle chemotherapy comprises the administration of a second cycle within about 60 days or about 3 months. In some instances, the method for multiple cycle chemotherapy comprises the administration of a second cycle within 50 days. In another instance, the second cycle is administered within 45, 40, 35, 30, 25, 21, 20, 15, 14, 10, 9, 8, 7, 6, 5, 4, 3, 2 or 1 day(s) of the first cycle. In some embodiments, the administration of any additional cycles is within 50 days of the previous cycle. In some embodiments, the administration of any additional cycles is within 10 days of the previous cycle. In some embodiments, the administration of any additional cycles is within 9 days of the previous cycle. In some embodiments, the administration of any additional cycles is within 8 days of the previous cycle. In some embodiments, the administration of any additional cycles is within 7 days of the previous cycle. In some embodiments, the administration of any additional cycles is within 6 days of the previous cycle. In some embodiments, the administration of any additional cycles is within 5 days of the previous cycle. In some embodiments, the administration of any additional cycles is within 4 days of the previous cycle. In some embodiments, the administration of any additional cycles is within 3 days of the previous cycle. In some embodiments, the administration of any additional cycles is within 2 days of the previous cycle. In some embodiments, the administration of any additional cycles is within 1 day of the previous cycle. In another embodiment, the additional cycle is administered within 45, 40, 35, 30, 25, 21, 20, 15, 14, 10, 9, 8, 7, 6, 5, 4, 3, 2, or 1 days of the previous cycle.

The length of a treatment cycle depends on the treatment being given. In some embodiments, the length of a treatment cycle ranges from two to six weeks. In some embodiments, the length of a treatment cycle ranges from four to six weeks. In some embodiments, the length of a treatment cycle is 28 days. In some embodiments, the length of a treatment cycle is 56 days. In some embodiments, a treatment cycle lasts one, two, three, or four weeks. In some embodiments, a treatment cycle lasts four weeks. The number of treatment doses scheduled within each cycle also varies depending on the drugs being given.

In certain instances, the compound of Formula (I), or an isotopic variant thereof; or a pharmaceutically acceptable salt, solvate, hydrate, or prodrug thereof, is administered to the subject on a 28-day cycle. In some embodiments, the compound of Formula (I), or an isotopic variant thereof; or a pharmaceutically acceptable salt, solvate, hydrate, or prodrug thereof, is administered to the subject for at least one 28-day cycle. In some embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for at least two 28-day cycles.

In certain embodiments, the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject for a period of up to about 7 days. In some embodiments, the days over which the compound of Formula (I), or an enantiomer, a mixture of enantiomers, a mixture of two or more diastereomers, or an isotopic variant thereof; or a pharmaceutically acceptable salt, solvate, hydrate, or prodrug thereof are intermittent. In some embodiments, administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof for about 7 consecutive days in a 28-day cycle.

In some embodiments, the method comprises an intermittent dosing schedule (IS), comprising administering to subject the compound of Formula (I), or an enantiomer, a mixture of enantiomers, a mixture of two or more diastereomers, or an isotopic variant thereof, or a pharmaceutically acceptable salt, solvate, hydrate, or prodrug thereof once daily for 7 consecutive days followed by 21 days without treatment in a 28-day cycle. In some embodiments, the compound of Formula (I), or an enantiomer, a mixture of enantiomers, a mixture of two or more diastereomers, or an isotopic variant thereof, or a pharmaceutically acceptable salt, solvate, hydrate, or prodrug thereof, is administered to the subject for at least one 28-day cycle. In some embodiments, the IS avoids or reduces adverse or unwanted side effects associated with the use of the PI3K inhibitor, such as enterocolitis (manifested as diarrhea), cutaneous toxicities, liver toxicity (manifested as elevation of transaminases), pulmonary toxicity (manifested as non-infectious pneumonitis), and infections. In some embodiments, the IS avoids or reduces enterocolitis, rash, transaminitis, or combinations thereof.

In some embodiments, the method comprises a continuous daily dosing schedule (CS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 28 consecutive days in a 28-day cycle. In some embodiments, the compound of Formula (I), or an enantiomer, a mixture of enantiomers, a mixture of two or more diastereomers, or an isotopic variant thereof, or a pharmaceutically acceptable salt, solvate, hydrate, or prodrug thereof, is administered to the subject for at least two CS 28-day cycles. In certain instances, the method comprises a continuous daily dosing schedule (CS) for at least two CS 28-day cycles, followed by an intermittant dosing schedule (IS), comprising administering to subject the compound of Formula (I), or an enantiomer, a mixture of enantiomers, a mixture of two or more diastereomers, or an isotopic variant thereof; or a pharmaceutically acceptable salt, solvate, hydrate, or prodrug thereof once daily for 7 consecutive days followed by 21 days without treatment in a 28-day cycle after the at least two CS 28-day cycles. In some embodiments, the dosing schedule avoids or reduces adverse or unwanted side effects associated with the use of the PI3K inhibitor, such as enterocolitis (manifested as diarrhea), cutaneous toxicities, liver toxicity (manifested as elevation of transaminases), pulmonary toxicity (manifested as non-infectious pneumonitis), and infections. In some embodiments, the dosing schedule avoids or reduces enterocolitis, rash, transaminitis, or combinations thereof.

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two cycles of continuous daily administration (CS) followed by daily administration for only the first seven days of each subsequent (IS) cycle, the CS and IS cycles are 28-day cycles, wherein the IS cycle is repeated until disease regression is no longer observed. In some or additional embodiments, if disease progression is observed in the subject, the subject resumes the 28-day cycles of continuous daily administration (CS) until disease regression or stabilization are observed

In certain instances of the treatment regimen comprising administration of the compound of Formula (I), or an enantiomer, a mixture of enantiomers, a mixture of two or more diastereomers, or an isotopic variant thereof; or a pharmaceutically acceptable salt, solvate, hydrate, or prodrug thereof for two 28-day cycles of continuous daily administration (CS) followed by daily administration for only the first seven days of each subsequent (IS) 28-day cycle; wherein disease regression or stabilization is no longer observed in the subject on the intermittent dosing schedule (IS) cycle, the subject resumes 28-day cycles of continuous daily administration (CS) until disease regression or stabilization are observed.

In some embodiments, the compound of Formula (I) is Compound I or an isotopic variant, pharmaceutically acceptable salt, solvate, hydrate, or prodrug thereof. In some embodiments, the compound of Formula (I) is Compound II or an isotopic variant, pharmaceutically acceptable salt, solvate, hydrate, or prodrug thereof. In some embodiments, the compound of Formula (I) is Compound III or an isotopic variant, pharmaceutically acceptable salt, solvate, hydrate, or prodrug thereof. In some embodiments, the compound of Formula (I) is Compound IV or an isotopic variant, pharmaceutically acceptable salt, solvate, hydrate, or prodrug thereof. In some embodiments, the compound of Formula (I) is Compound V or an isotopic variant, pharmaceutically acceptable salt, solvate, hydrate, or prodrug thereof. In some embodiments, the compound of Formula (I) is Compound VI or an isotopic variant, pharmaceutically acceptable salt, solvate, hydrate, or prodrug thereof. In some embodiments, the compound of Formula (I) is Compound VII or an isotopic variant, pharmaceutically acceptable salt, solvate, hydrate, or prodrug thereof. In some embodiments, the compound of Formula (I) is Compound VIII or an isotopic variant, pharmaceutically acceptable salt, solvate, hydrate, or prodrug thereof. In some embodiments, the compound of Formula (I) is Compound IX or an isotopic variant, pharmaceutically acceptable salt, solvate, hydrate, or prodrug thereof. In some embodiments, the compound of Formula (I) is Compound X or an isotopic variant, pharmaceutically acceptable salt, solvate, hydrate, or prodrug thereof. In some embodiments, the compound of Formula (I) is Compound XI or an isotopic variant, pharmaceutically acceptable salt, solvate, hydrate, or prodrug thereof. In some embodiments, the compound of Formula (I) is Compound XII or an isotopic variant, pharmaceutically acceptable salt, solvate, hydrate, or prodrug thereof. In some embodiments, the compound of Formula (I) is Compound XIII or an isotopic variant, pharmaceutically acceptable salt, solvate, hydrate, or prodrug thereof. In some embodiments, the compound of Formula (I) is Compound XIV or an isotopic variant, pharmaceutically acceptable salt, solvate, hydrate, or prodrug thereof. In some embodiments, the compound of Formula (I) is Compound XV or an isotopic variant, pharmaceutically acceptable salt, solvate, hydrate, or prodrug thereof. In some embodiments, the compound of Formula (I) is Compound XVI or an isotopic variant, pharmaceutically acceptable salt, solvate, hydrate, or prodrug thereof.

The suitability of a particular route of administration employed for a particular active agent will depend on the active agent itself (e.g., whether it can be administered orally without decomposing prior to entering the blood stream) and the disease being treated. Recommended routes of administration for the second active agents are known to those of ordinary skill in the art. See, e.g., Physicians' Desk Reference, 1755-1760 (56th ed., 2002).

In certain embodiments, the compound of Formula (I), or an isotopic variant thereof, or a pharmaceutically acceptable salt, solvate, hydrate, or prodrug thereof and a BTK inhibitor are administered simultaneously, at essentially the same time, or sequentially. If administration takes place sequentially, the BTK inhibitor may be administered before or after administration of a compound of Formula (I), or an isotopic variant thereof; or a pharmaceutically acceptable salt, solvate, hydrate, or prodrug thereof. In some embodiments, the BTK inhibitor is administered before administration of a compound of Formula (I), or an isotopic variant thereof, or a pharmaceutically acceptable salt, solvate, hydrate, or prodrug thereof. In some embodiments, the BTK inhibitor is administered simultaneously with administration of a compound of Formula (I), an isotopic variant thereof; or a pharmaceutically acceptable salt, solvate, hydrate, or prodrug thereof. In some embodiments, the BTK inhibitor is administered after the administration of a compound of Formula (I), an isotopic variant thereof; or a pharmaceutically acceptable salt, solvate, hydrate, or prodrug thereof. In some embodiments, a compound of Formula (I), or an isotopic variant thereof; or a pharmaceutically acceptable salt, solvate, hydrate, or prodrug thereof and the BTK inhibitor need not be administered by means of the same vehicle. In some embodiments, the BTK inhibitor and a compound of Formula (I), or an isotopic variant thereof; or a pharmaceutically acceptable salt, solvate, hydrate, or prodrug thereof are administered in different vehicles. The BTK inhibitor may be administered one or more times, and the number of administrations of each component of the combination may be the same or different. In addition, a compound of Formula (I), or an isotopic variant thereof, or a pharmaceutically acceptable salt, solvate, hydrate, or prodrug thereof and the BTK inhibitor need not be administered at the same site.

In certain embodiments, a compound of Formula (I), or an isotopic variant thereof, or a pharmaceutically acceptable salt, solvate, hydrate, or prodrug thereof and the BTK inhibitor are cyclically administered to a patient. Cycling therapy involves the administration of an active agent or a combination of active agents for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment.

In some embodiments, the compound of Formula (I) is administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the compound of Formula (I) is administered daily. In some embodiments, the compound of Formula (I) is administered daily for a period of up to about 28 days. In some embodiments, the compound of Formula (I) is administered daily for a period of up to about 7 days.

In some embodiments, the BTK inhibitor is administered daily, every other day, every other day 3 times a week, every 3 days, every 4 days, every 5 days, every 6 days, weekly, every 2 weeks, every 3 weeks, every 4 weeks, every 5 weeks, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the BTK inhibitor is administered 8 times in 6 months.

In some instances, the compound of Formula (I) or the BTK inhibitor is optionally given continuously; alternatively, the dose of drug being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 8 days, 9 days, 10 days, 12 days, 14 days, 15 days, 20 days, 21 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In certain embodiments, in the treatment, prevention, or amelioration of one or more symptoms of the disorders, diseases, or conditions described herein, an appropriate dosage level of a compound of Formula (I), or an isotopic variant thereof; or a pharmaceutically acceptable salt, solvate, hydrate, or prodrug thereof generally is ranging from about 1 to 1000 mg, from about 1 to about 500 mg, from about 5 to about 500 mg, from about 5 to about 200 mg, from about 5 to about 250 mg or from about 10 to about 150 mg which can be administered in single or multiple doses. In certain embodiments, the compound of Formula (I), or an isotopic variant thereof; or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 450 or 500 mg. In certain embodiments, the compound of Formula (I), or an isotopic variant thereof; or a pharmaceutically acceptable salt, solvate, hydrate, or prodrug thereof is administered in an amount of about 60 mg, about 120 mg, about 150 mg, or about 180 mg. In certain embodiments, the compound of Formula (I), or an isotopic variant thereof; or a pharmaceutically acceptable salt, solvate, hydrate, or prodrug thereof is administered in an amount of about 30 mg. In certain embodiments, the compound of Formula (I), or an isotopic variant thereof; or a pharmaceutically acceptable salt, solvate, hydrate, or prodrug thereof is administered in an amount of about 45 mg. In certain embodiments, the compound of Formula (I), or an isotopic variant thereof; or a pharmaceutically acceptable salt, solvate, hydrate, or prodrug thereof is administered in an amount of about 60 mg. In certain embodiments, the compound of Formula (I), or an isotopic variant thereof; or a pharmaceutically acceptable salt, solvate, hydrate, or prodrug thereof is administered in an amount of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 225, about 250, about 275, about 300, about 325, about 350, about 375, about 400, about 450, or about 500 mg. In certain embodiments, the compound of Formula (I), or an isotopic variant thereof, or a pharmaceutically acceptable salt, solvate, hydrate, or prodrug thereof is administered in an amount of about 90 mg. In certain embodiments, the compound of Formula (I), or an isotopic variant thereof; or a pharmaceutically acceptable salt, solvate, hydrate, or prodrug thereof is administered in an amount of about 120 mg. In certain embodiments, the compound of Formula (I), or an isotopic variant thereof; or a pharmaceutically acceptable salt, solvate, hydrate, or prodrug thereof is administered in an amount of about 150 mg. In certain embodiments, the compound of Formula (I), or an isotopic variant thereof; or a pharmaceutically acceptable salt, solvate, hydrate, or prodrug thereof is administered in an amount of about 180 mg.

In certain embodiments, the compound of Formula (I), or an isotopic variant thereof; or a pharmaceutically acceptable salt, solvate, hydrate, or prodrug thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 450 or 500 mg/day. In certain embodiments, the compound of Formula (I), or an isotopic variant thereof; or a pharmaceutically acceptable salt, solvate, hydrate, or prodrug thereof is administered in an amount of about 45 mg/day. In certain embodiments, the compound of Formula (I), or an isotopic variant thereof; or a pharmaceutically acceptable salt, solvate, hydrate, or prodrug thereof is administered in an amount of about 60 mg/day. In certain embodiments, the compound of Formula (I), or an isotopic variant thereof; or a pharmaceutically acceptable salt, solvate, hydrate, or prodrug thereof is administered in an amount of about 90 mg/day. In certain embodiments, the compound of Formula (I), or an isotopic variant thereof; or a pharmaceutically acceptable salt, solvate, hydrate, or prodrug thereof is administered in an amount of about 120 mg/day. In certain embodiments, the compound of Formula (I), or an isotopic variant thereof; or a pharmaceutically acceptable salt, solvate, hydrate, or prodrug thereof is administered in an amount of about 150 mg/day. In certain embodiments, the compound of Formula (I), or an isotopic variant thereof; or a pharmaceutically acceptable salt, solvate, hydrate, or prodrug thereof is administered in an amount of about 180 mg/day.

For oral administration, the pharmaceutical compositions provided herein can be formulated in the form of tablets or capsules containing from about 1.0 to about 1,000 mg of a compound of Formula (I), or an isotopic variant thereof; or a pharmaceutically acceptable salt, solvate, hydrate, or prodrug thereof, in one embodiment, about 1, about 5, about 10, about 15, about 20, about 25, about 30, about 45, about 50, about 60, about 75, about 90, about 100, about 120, about 150, about 180, about 200, about 250, about 300, about 400, about 500, about 600, about 750, about 800, about 900, and about 1,000 mg of the a compound of Formula (I), or an isotopic variant thereof, or a pharmaceutically acceptable salt, solvate, hydrate, or prodrug thereof for the symptomatic adjustment of the dosage to the patient to be treated.

In some embodiments, the pharmaceutical compositions provided herein can be formulated in the form of tablets containing about 45, 60, 90, 120, 150, or 180 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In some embodiments, the compound of Formula (I), or an isotopic variant thereof; or a pharmaceutically acceptable salt, solvate, hydrate, or prodrug thereof is administered once per day. In some embodiments, about 30 mg, about 45 mg, or about 60 mg of the compound of Formula (I), or an isotopic variant thereof, or a pharmaceutically acceptable salt, solvate, hydrate, or prodrug thereof is administered once per day.

In certain embodiments, a compound of Formula (I), or an isotopic variant thereof; or a pharmaceutically acceptable salt, solvate, hydrate, or prodrug thereof is administered to a patient in need thereof in an amount of about 45 mg daily for 28 days or 56 days. In certain specific embodiments, a compound of Formula (I), or an isotopic variant thereof, or a pharmaceutically acceptable salt, solvate, hydrate, or prodrug thereof is administered to a patient in need thereof in an amount of about 45 mg daily for 28 days. In other specific embodiments, a compound of Formula (I), or an isotopic variant thereof, or a pharmaceutically acceptable salt, solvate, hydrate, or prodrug thereof is administered to a patient in need thereof in an amount of about 45 mg daily for 56 days.

In some embodiments, the pharmaceutical compositions provided herein can be formulated in the form of tablets containing about 60 mg of a compound of Formula (I), or an isotopic variant thereof, or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 60 mg daily for 28 days or 56 days. In certain specific embodiments, a compound of Formula (I), or an isotopic variant thereof, or a pharmaceutically acceptable salt, solvate, hydrate, or prodrug thereof is administered to a patient in need thereof in an amount of about 60 mg daily for 28 days. In other specific embodiments, a compound of Formula (I), or an isotopic variant thereof, or a pharmaceutically acceptable salt, solvate, hydrate, or prodrug thereof is administered to a patient in need thereof in an amount of about 60 mg daily for 56 days.

In some embodiments, the pharmaceutical compositions provided herein can be formulated in the form of tablets containing about 90 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 90 mg daily for 28 days or 56 days. In certain specific embodiments, a compound of Formula (I), or an isotopic variant thereof, or a pharmaceutically acceptable salt, solvate, hydrate, or prodrug thereof is administered to a patient in need thereof in an amount of about 90 mg daily for 28 days. In other specific embodiments, a compound of Formula (I), or an isotopic variant thereof, or a pharmaceutically acceptable salt, solvate, hydrate, or prodrug thereof is administered to a patient in need thereof in an amount of about 90 mg daily for 56 days.

In some embodiments, the pharmaceutical compositions provided herein can be formulated in the form of tablets containing about 120 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 120 mg daily for 28 days or 56 days. In certain specific embodiments, a compound of Formula (I), or an isotopic variant thereof, or a pharmaceutically acceptable salt, solvate, hydrate, or prodrug thereof is administered to a patient in need thereof in an amount of about 120 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 120 mg daily for 56 days.

In some embodiments, the pharmaceutical compositions provided herein can be formulated in the form of tablets containing about 150 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 150 mg daily for 28 days or 56 days. In certain specific embodiments, a compound of Formula (I), or an isotopic variant thereof, or a pharmaceutically acceptable salt, solvate, hydrate, or prodrug thereof is administered to a patient in need thereof in an amount of about 150 mg daily for 28 days. In other specific embodiments, a compound of Formula (I), or an isotopic variant thereof, or a pharmaceutically acceptable salt, solvate, hydrate, or prodrug thereof is administered to a patient in need thereof in an amount of about 150 mg daily for 56 days.

In some embodiments, the pharmaceutical compositions provided herein can be formulated in the form of tablets containing about 180 mg of a compound of Formula (I), or an isotopic variant thereof; or a pharmaceutically acceptable salt, solvate, hydrate, or prodrug thereof. The pharmaceutical compositions can be administered on a regimen of 1 to 4 times per day, including once, twice, three times, and four times per day. In certain embodiments, a compound of Formula (I), or an isotopic variant thereof; or a pharmaceutically acceptable salt, solvate, hydrate, or prodrug thereof is administered to a patient in need thereof in an amount of about 180 mg daily for 28 days or 56 days. In certain specific embodiments, a compound of Formula (I), or an isotopic variant thereof; or a pharmaceutically acceptable salt, solvate, hydrate, or prodrug thereof is administered to a patient in need thereof in an amount of about 180 mg daily for 28 days. In other specific embodiments, a compound of Formula (I), or an isotopic variant thereof; or a pharmaceutically acceptable salt, solvate, hydrate, or prodrug thereof is administered to a patient in need thereof in an amount of about 180 mg daily for 56 days.

In the methods of treatment, prevention, or amelioration of one or more symptoms of the disorders, diseases, or conditions described herein, an appropriate dosage level of a BTK inhibitor generally is ranging from about 0.1 to 2000 milligrams per day. For example, 1-500 milligrams once or multiple times per day may be effective to obtain the desired results.

In certain embodiments, the BTK inhibitor is ibrutinib and the amount of ibrutinib that is administered is from about 10 mg/day up to, and including, 1000 mg/day. In certain embodiments, the amount of ibrutinib that is administered is from about 10 mg/day to 600 mg/day. In certain embodiments, the amount of ibrutinib that is administered is from about 100 mg/day to 600 mg/day. In certain embodiments, the amount of ibrutinib that is administered per day is about 10 mg, about 50 mg, about 100 mg, about 140 mg, about 280 mg, about 420 mg or about 560 mg.

In certain embodiments, the BTK inhibitor is BGB-3111 and the amount of BGB-3111 that is administered is from about 10 mg/day up to, and including, 1000 mg/day. In certain embodiments, the amount of BGB-3111 that is administered is from about 10 mg/day to 600 mg/day. In certain embodiments, the amount of BGB-3111 that is administered is from about 100 mg/day to 600 mg/day. In certain embodiments, the amount of BGB-3111 that is administered per day is about 10 mg, about 30 mg, about 45 mg, about 50 mg, about 100 mg, about 150 mg, about 160 mg, about 170 mg, about 180 mg, about 190 mg, about 200 mg, about 220 mg, about 240 mg, about 260 mg, about 280 mg, about 300 mg, about 320 mg, about 340 mg, about 360 mg, about 400 mg, about 440 mg, about 480 mg, about 520 mg, or about 560 mg.

In certain embodiments, the BTK inhibitor, or a pharmaceutically acceptable salt thereof, is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 365, 370, 375, 400, 450, 500, or 560 mg. In certain embodiments, the BTK inhibitor, or a pharmaceutically acceptable salt thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 365, 370, 375, 400, 450, 500, or 560 mg/day. In certain embodiments, the BTK inhibitor, or a pharmaceutically acceptable salt thereof, is administered in an amount of about 160 mg. In certain embodiments, the BTK inhibitor, or a pharmaceutically acceptable salt thereof, is administered in an amount of about 320 mg. In certain embodiments, the BTK inhibitor or a pharmaceutically acceptable salt thereof is administered in an amount of about 10 mg, about 30 mg, about 45 mg, about 50 mg, about 100 mg, about 150 mg, about 160 mg, about 170 mg, about 180 mg, about 190 mg, about 200 mg, about 220 mg, about 240 mg, about 260 mg, about 280 mg, about 300 mg, about 320 mg, about 340 mg, about 360 mg, about 400 mg, about 440 mg, about 480 mg, about 520 mg, or about 560 mg. In certain embodiments, the BTK inhibitor, or a pharmaceutically acceptable salt thereof, is administered in an amount of about 80 mg. In certain embodiments, the BTK inhibitor, or a pharmaceutically acceptable salt thereof, is administered in an amount of about 160 mg. In certain embodiments, the BTK inhibitor, or a pharmaceutically acceptable salt thereof, is administered in an amount of about 200 mg. In certain embodiments, the BTK inhibitor, or a pharmaceutically acceptable salt thereof, is administered in an amount of about 240 mg. In certain embodiments, the BTK inhibitor, or a pharmaceutically acceptable salt thereof, is administered in an amount of about 280 mg. In certain embodiments, the BTK inhibitor, or a pharmaceutically acceptable salt thereof, is administered in an amount of about 320 mg. In certain embodiments, the BTK inhibitor, or a pharmaceutically acceptable salt thereof, is administered in an amount of about 360 mg. In certain embodiments, the BTK inhibitor, or a pharmaceutically acceptable salt thereof, is administered in an amount of about 400 mg. In certain embodiments, the BTK inhibitor, or a pharmaceutically acceptable salt thereof, is administered in an amount of about 440 mg. In certain embodiments, the BTK inhibitor, or a pharmaceutically acceptable salt thereof, is administered in an amount of about 480 mg. In certain embodiments, the BTK inhibitor, or a pharmaceutically acceptable salt thereof, is administered in an amount of about 520 mg. In certain embodiments, the BTK inhibitor, or a pharmaceutically acceptable salt thereof, is administered in an amount of about 560 mg.

In some embodiments, about 30 mg of the compound of Formula (I), or an isotopic variant thereof; or a pharmaceutically acceptable salt, solvate, hydrate, or prodrug thereof is administered once per day and about 160 mg of the BTK inhibitor, or a pharmaceutically acceptable salt thereof is administered twice per day. In some embodiments, about 30 mg of the compound of Formula (I), or an isotopic variant thereof; or a pharmaceutically acceptable salt, solvate, hydrate, or prodrug thereof is administered once per day and about 160 mg of the BTK inhibitor, or a pharmaceutically acceptable salt thereof is administered twice per day.

In some embodiments, about 45 mg of the compound of Formula (I), or an isotopic variant thereof; or a pharmaceutically acceptable salt, solvate, hydrate, or prodrug thereof is administered once per day and about 160 mg of the BTK inhibitor, or a pharmaceutically acceptable salt thereof is administered twice per day. In some embodiments, about 45 mg of the compound of Formula (I), or an isotopic variant thereof; or a pharmaceutically acceptable salt, solvate, hydrate, or prodrug thereof is administered once per day and about 160 mg of the BTK inhibitor, or a pharmaceutically acceptable salt thereof is administered twice per day.

In some embodiments, about 60 mg of the compound of Formula (I), or an isotopic variant thereof; or a pharmaceutically acceptable salt, solvate, hydrate, or prodrug thereof is administered once per day and about 160 mg of the BTK inhibitor, or a pharmaceutically acceptable salt thereof is administered twice per day. In some embodiments, about 60 mg of the compound of Formula (I), or an isotopic variant thereof; or a pharmaceutically acceptable salt, solvate, hydrate, or prodrug thereof is administered once per day and about 160 mg of the BTK inhibitor, or a pharmaceutically acceptable salt thereof is administered twice per day.

For oral administration, the pharmaceutical compositions provided herein can be formulated in the form of tablets or capsules containing from about 1.0 to about 1,000 mg of a BTK inhibitor, or a pharmaceutically acceptable salt thereof, in one embodiment, about 1, about 25, about 50, about 100, about 125, about 150, about 160, about 170, about 180, about 190, about 200, about 300, about 310, about 320, about 330, about 340, about 350, about 360, about 370, about 380, about 390, about 400, about 500, about 600, about 700, and about 1,000 mg of the BTK inhibitor, or pharmaceutically acceptable salt thereof for the symptomatic adjustment of the dosage to the patient to be treated. The pharmaceutical compositions can be administered on a regimen of 1 to 4 times per day, including once, twice, three times, and four times per day. In certain embodiments, the BTK inhibitor is administered once per day, twice per day, three times per day, or four times per day. In certain embodiments, the BTK inhibitor is administered once per day. In certain embodiments, the BTK inhibitor is administered twice per day. In some embodiments, about 320 mg of the BTK inhibitor, or a pharmaceutically acceptable salt thereof is administered once per day. In some embodiments, about 160 mg of the BTK inhibitor, or a pharmaceutically acceptable salt thereof is administered twice per day.

In certain embodiments, the BTK inhibitor is co-administered (e.g., in a single dosage form), once per day. In certain embodiments, the BTK inhibitor is co-administered (e.g., in a single dosage form), twice per day.

In certain embodiments, about 30 mg, about 45 mg, or about 60 mg of the compound of Formula (I), or an isotopic variant thereof; or a pharmaceutically acceptable salt, solvate, hydrate, or prodrug thereof is administered once per day and about 160 mg of a BTK inhibitor (e.g., BGB-3111) is administered twice per day. In certain embodiments, about 30 mg, about 45 mg, or about 60 mg of the compound of Formula (I), or an isotopic variant thereof; or a pharmaceutically acceptable salt, solvate, hydrate, or prodrug thereof is administered once per day and about 320 mg of a BTK inhibitor (e.g., BGB-3111) is administered once per day.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Additional Combination Therapy

In certain embodiments, the methods of combination therapy comprising a compound of Formula (I) an isotopic variant thereof; or a pharmaceutically acceptable salt, solvate, hydrate, or prodrug thereof and a BTK inhibitor can also be combined or used in combination with a third agent or therapies useful in the treatment, prevention, or amelioration of one or more symptoms of a proliferative disorders, diseases, or conditions.

Suitable third agent of therapies include, but are not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and xirnelagatran, (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma monists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid secjuestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) famesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralcorticoidreceptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat, and (54) other miscellaneous agents, such as, hydroxyurea, procarbazine, mitotane, hexamethylmelamine, and gold compounds.

In certain embodiments, the third therapies that may be used in combination with the methods provided herein include, but are not limited to, surgery, endocrine therapy, biologic response modifiers (e.g., interferons, interleukins, and tumor necrosis factor (TNF)), hyperthermia and cryotherapy, and agents to attenuate any adverse effects (e.g., antiemetics).

In certain embodiments, the third therapeutic agents that may be used in combination with the compounds provided herein include, but are not limited to, alkylating drugs (mechlorethamine, chlorambucil, cyclophosphamide, melphalan, and ifosfamide), antimetabolites (cytarabine (also known as cytosine arabinoside or Ara-C), and methotrexate), purine antagonists and pyrimidine antagonists (6-mercaptopurine, 5-fluorouracil, cytarbine, and gemcitabine), spindle poisons (vinblastine, vincristine, and vinorelbine), podophyllotoxins (etoposide, irinotecan, and topotecan), antibiotics (daunorubicin, doxorubicin, bleomycin, and mitomycin), nitrosoureas (carmustine and lomustine), enzymes (asparaginasc), and hormones (tamoxifen, leuprolide, flutamide, and megestrol), imatinib, adriamycin, dexamethasone, and cyclophosphamide. For a more comprehensive discussion of updated cancer therapies; See, http://www.nci.nih.gov/, a list of the FDA approved oncology drugs at http://www.fda.gov/cder/cancer/dniglistframe.htm, and The Merck Manual, Seventeenth Ed. 1999, the entire contents of which are hereby incorporated by reference.

In another embodiment, the methods provided herein comprise administration of a compound of Formula (I), or an isotopic variant thereof, or a pharmaceutically acceptable salt, solvate, hydrate, or prodrug thereof and a BTK inhibitor, together with administration of one or more chemotherapeutic agents and/or therapies selected from: alkylation agents (e.g., cisplatin, carboplatin); antimetabolites (e.g., methotrexate and 5-FU); antitumor antibiotics (e.g., adriamymycin and bleomycin); antitumor vegetable alkaloids (e.g., taxol and etoposide); antitumor hormones (e.g., dexamethasone and tamoxifen); antitumor immunological agents (e.g., interferon α, β, and γ); radiation therapy; and surgery. In certain embodiments, the one or more chemotherapeutic agents and/or therapies are administered to the subject before, during, or after the administration of a compound of Formula (I), or an isotopic variant thereof, or a pharmaceutically acceptable salt, solvate, hydrate, or prodrug thereof and a BTK inhibitor.

Such other agents, or drugs, can be administered, by a route and in an amount commonly used therefor, simultaneously or sequentially with a compound of Formula (I), or an isotopic variant thereof; or a pharmaceutically acceptable salt, solvate, hydrate, or prodrug thereof and a BTK inhibitor. When a compound of Formula (I) and a BTK inhibitor are used contemporaneously with one or more other drugs, a pharmaceutical composition containing such other drugs in addition to the a compound of Formula (I), or an isotopic variant thereof; or a pharmaceutically acceptable salt, solvate, hydrate, or prodrug thereof and a BTK inhibitor can be utilized, but is not required. Accordingly, the pharmaceutical compositions provided herein include those that also contain one or more other active ingredients or therapeutic agents, in addition to a compound of Formula (I).

### Pharmaceutical Compositions and Routes of Administration

Provided herein is a pharmaceutical composition comprising a compound provided herein (a compound of Formula (I) and/or a BTK inhibitor) and a pharmaceutically acceptable excipient, adjuvant, carrier, buffer, or stabilizer. In some embodiments, the compound of Formula (I) and the BTK inhibitor are present in the same pharmaceutical composition. In some embodiments, the compound of Formula (I) and the BTK inhibitor are in different pharmaceutical compositions.

In one embodiment, the pharmaceutical compositions are provided in a dosage form for oral administration, which comprise a compound provided herein, and one or more pharmaceutically acceptable excipients or carriers. The pharmaceutical compositions provided herein that are formulated for oral administration may be in tablet, capsule, powder, or liquid form. In some embodiments, a tablet comprises a solid carrier or an adjuvant. Liquid pharmaceutical compositions generally comprise a liquid carrier such as water, petroleum, animal or vegetable oils, mineral oil, or synthetic oil. Physiological saline solution, dextrose or other saccharide solution, or glycols such as ethylene glycol, propylene glycol, or polyethylene glycol may be included. In some embodiments, a capsule comprises a solid carrier such as gelatin.

In another embodiment, the pharmaceutical compositions are provided in a dosage form for parenteral administration, which comprise a compound provided herein, and one or more pharmaceutically acceptable excipients or carriers. Where pharmaceutical compositions may be formulated for intravenous, cutaneous or subcutaneous injection, the active ingredient will be in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. In some embodiments, preservatives, stabilisers, buffers, antioxidants, and/or other additives are included as required.

In yet another embodiment, the pharmaceutical compositions are provided in a dosage form for topical administration, which comprise a compound provided herein, and one or more pharmaceutically acceptable excipients or carriers.

The pharmaceutical compositions can also be formulated as modified release dosage forms, including delayed-, extended-, prolonged-, sustained-, pulsatile-, controlled-, accelerated-, fast-, targeted-, and programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd Edition, Rathbone et al., Eds., Marcel Dekker, Inc.: New York, N.Y., 2008).

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In certain embodiments, the pharmaceutical compositions provided herein further comprise one or more chemotherapeutic agents as defined herein.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SILO (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SILO (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-O-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungi static concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and metliacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

Provided herein also are kits which, when used by the medical practitioner, can simplify the administration of appropriate amounts of active ingredients to a subject. In certain embodiments, the kit provided herein includes one or more containers and a dosage form of a compound of Formula (I), or an isotopic variant thereof; or a pharmaceutically acceptable salt, solvate, hydrate, or prodrug thereof and a BTK inhibitor.

In certain embodiments, the kit provided herein includes one or more containers and a dosage form of a compound of Formula (I), or an isotopic variant thereof; or a pharmaceutically acceptable salt, solvate, hydrate, or prodrug thereof and a BTK inhibitor. Kits provided herein can further include devices that are used to administer the active ingredients. Examples of such devices include, but are not limited to, syringes and needle-less injectors drip bags.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### Examples

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); 4, (microliters); M (molar); mM (millimolar), μM (micro molar); eq. (equivalent); mmol (millimoles), Hz (Hertz), MHz (megahertz); hr or hrs (hour or hours); min (minutes); and MS (mass spectrometry).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated herein are intended to exemplify the applicable chemistry through the use of specific examples and are not indicative of the scope of the disclosure.

Synthesis of compound I is described in U.S. Pat. No. 9,056,852 B2, which is incorporated by reference for such disclosure.

### Example 1: Synthesis of 4-(2-(difluoromethyl)-1H-benzo[d]imidazol-1-yl)-N-(2-methyl-1-(2-(1-methylpiperidin-4-yl)phenyl)propan-2-yl)-6-morpholino-1,3,5-triazin-2-amine, Compound I

A mixture of 4-(2-(difluoromethyl)-1H-benzo[d]imidazol-1-yl)-N-(2-methyl-1-(2-(piperidin-4-yl)phenyl)propan-2-yl)-6-morpholino-1,3,5-triazin-2-amine (80 mg, 0.14 mmol), aq. formaldehyde (37%, 23 mg), and sodium cyanoborohydride (11 mg, 0.17 mmol) in methanol (2 mL) was stirred at room temperature for 1 hr. The crude product was purified by prep-HPLC to give compound I (11 mg, 13% yield) as a white solid: 99% purity (HPLC); MS m/z: 577.3 (M+1); ¹H NMR (CDCl₃, 500 MHz) δ 8.37 (d, 1H), 7.90 (d, 1H), 7.64 (t, 1H), 7.42 (m, 2H), 7.32 (d, 1H), 7.24 (1, 1H), 7.13 (t, 1H), 7.07 (d, 1H), 5.15 (s, 1H), 4.00-3.70 (m, 8H), 3.28 (s, 2H), 2.94 (m, 2H), 2.78 (m, 2H), 2.28 (s, 3H), 1.891.60 (m, 6H), 1.53 (s, 6H) ppm.

### Example 2: Combination Studies

The activity of compound I was examined in combination with PCI-32765 (ibrutinib), across a panel of 8 DLBCL cell lines indicated below (7 GCB subtype cell lines and 1 ABC subtype cell line).

TABLE 1

| Cell Lines | Subtype |
|---|---|
| DB | GCB |
| DOHH-2 | GCB |
| HT | GCB |
| NU-DHL-1 | GCB |
| Pfeiffer | GCB |
| SU-DHL-10 | GCB |
| OCI-Ly19 | GCB |
| OCI-Ly3 | ABC |

### ATPLite Assay

The effect of test compounds and combinations on growth inhibition as a measure of cell viability was determined in an ATPLite assay. The endpoint readout of the assay was based upon quantitation of ATP as an indicator of viable cells.

Cells were thawed from a liquid nitrogen preserved state. Once cells were expanded and divided at their expected doubling times, screening begins. Cells were seeded in growth media in black 384-well tissue culture treated plates at 500 cells per well (except where noted in Analyzer). Cells were equilibrated in assay plates via centrifugation and placed in incubators attached to the Dosing Modules at 37° C. for 24 hours before treatment. At the time of treatment, a set of assay plates (which do not receive treatment) were collected and ATP levels were measured by adding ATPLite (Perkin Elmer). These Tzero (T₀) plates were read using ultra-sensitive luminescence on Envision Plate Readers. Treated assay plates were incubated with compound for 72 hours. All data points were collected via automated processes; quality controlled; and analyzed using Horizon Discovery proprietary software. Assay plates were accepted if they pass the following quality control standards: relative luciferase values were consistent throughout the entire experiment, Z-factor scores were greater than 0.6, untreated/vehicle controls behaved consistently on the plate.

Growth Inhibition (GI) was used as a measure of cell viability. The cell viability of vehicle was measured at the time of dosing (T₀) and after seventy two hours (T₇₂). A GI reading of 0% represented no growth inhibition—cells treated with compound and T₇₂ or T₁₂₀ vehicle signals were matched. A GI 100% represented complete growth inhibition—cells treated by compound and Tₒ vehicle signals were matched. Cell numbers did not increase during the treatment period in wells with GI 100% and may suggest a cytostatic effect for compounds reaching a plateau at this effect level. A GI 200% represents complete death of all cells in the culture well. Compounds reaching an activity plateau of GI 200% were considered cytotoxic. GI was calculated by applying the following test and equation:

$$If T < {V}_{0} : 100 \cdot ( 1 - \frac{T - {V}_{0}}{{V}_{0}} )$$

$$lf T \geq {V}_{0} : 100 \cdot ( 1 - \frac{T - {V}_{0}}{V - {V}_{0}} )$$

where T is the signal measure for a test article, V is the vehicle-treated control measure, and V₀ is the vehicle control measure at time zero. This formula is derived from the Growth Inhibition calculation used in the National Cancer Institute's[sic] NCl[sic]-60 high throughput screen.

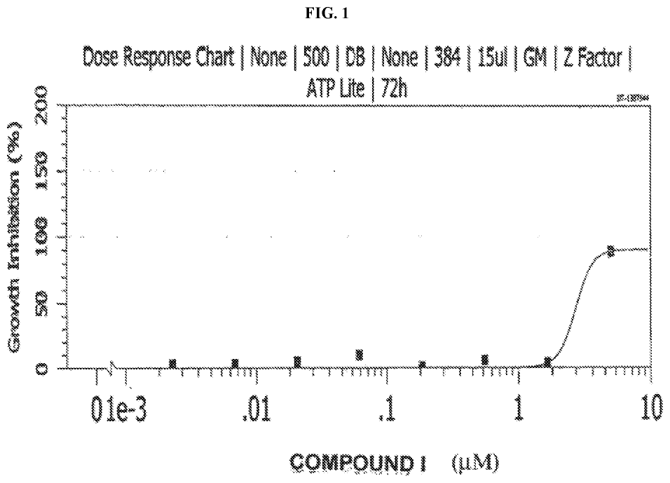

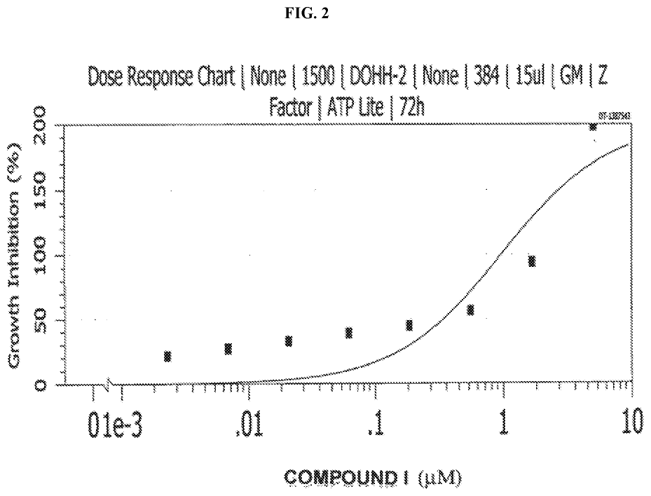

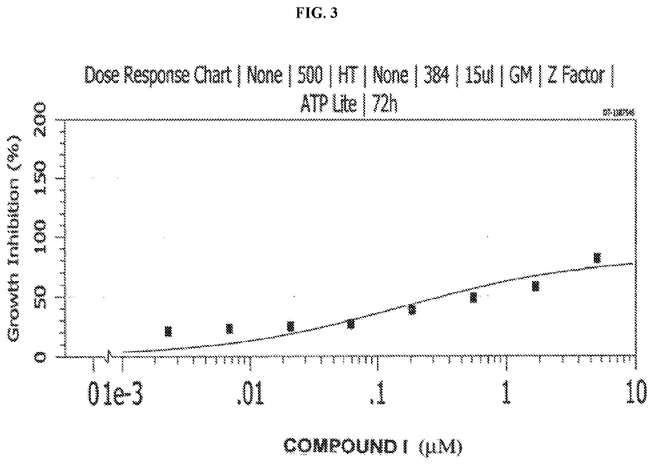

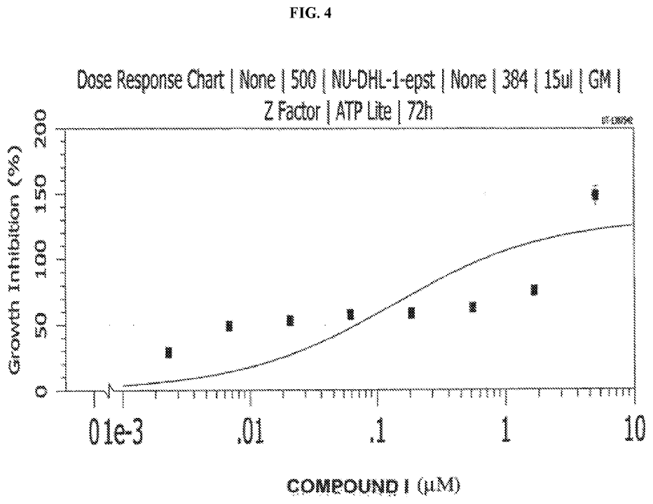

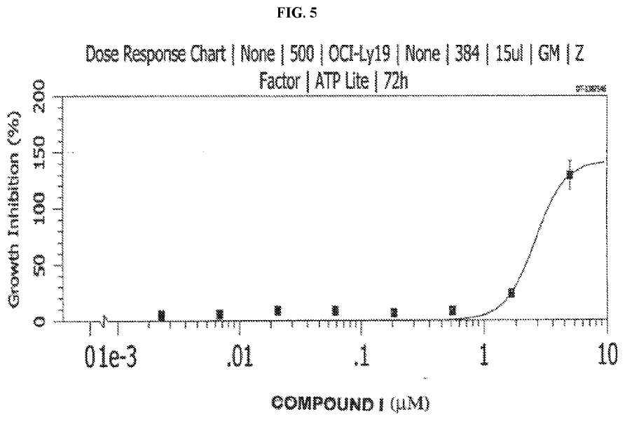

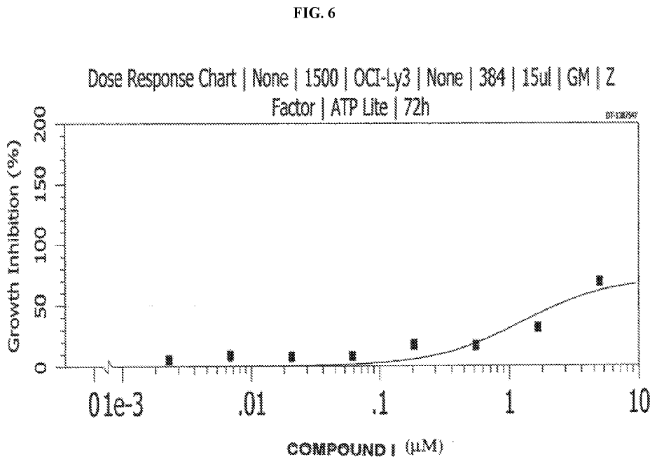

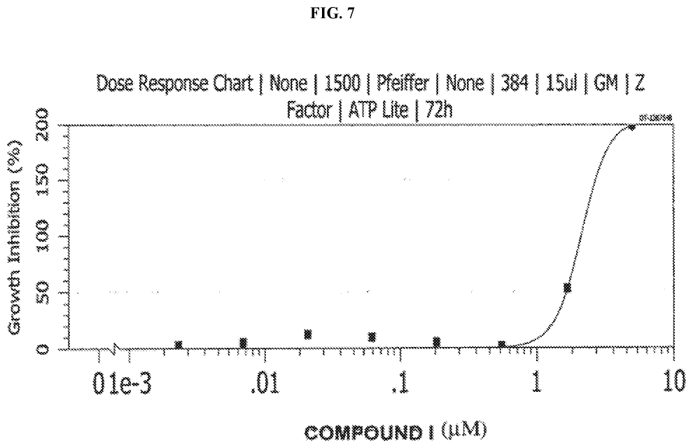

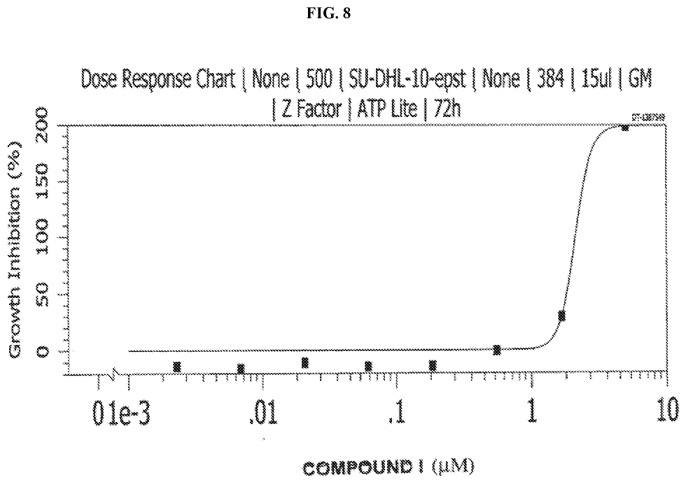

FIGS. 1-8 provide signal agent dose response curves for growth inhibition by compound I in the cell lines indicated in Table 1.

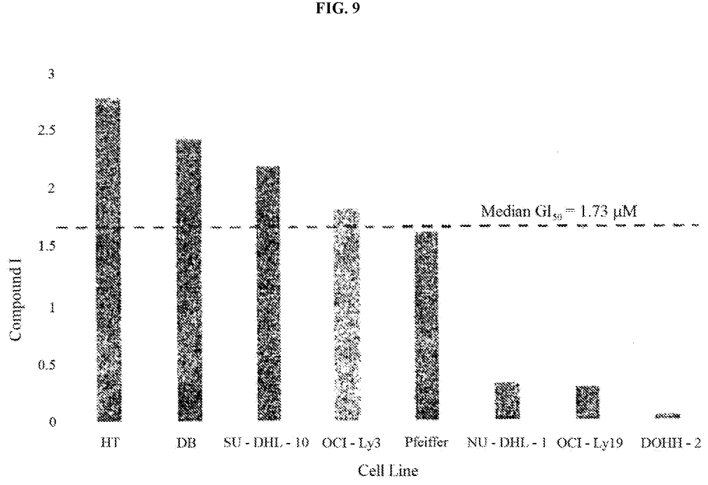

As illustrated in FIG. 9, compound I had varying activity levels across cell line panel. All cell lines achieved growth inhibition levels of >50% with median GI₅₀ of 1.73 μM.

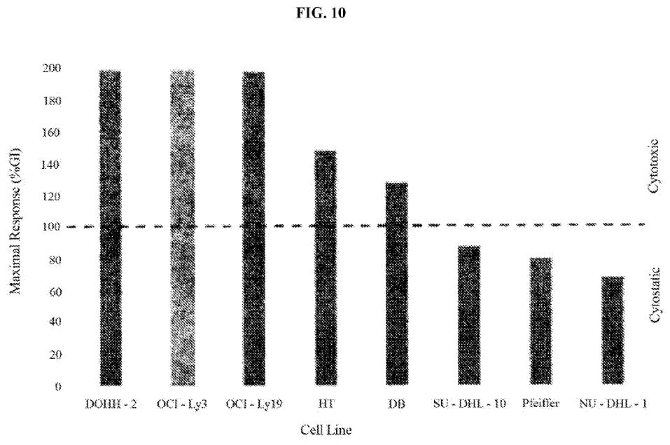

As illustrated in FIG. 10, compound I induced cytotoxic effects in 3 cell lines (DOHH-2, OCI-Ly3 and OCI-Ly19) and sub-cytostatic effects in 3 cell lines (SU-DIAL-10, Pfeiffer and NU-DHL-1).

Loewe Additivity model

Loewe Additivity model was used for measuring the effects of drug combinations. The Loewe additivity model is dose-based and applies only to the activity levels achieved by the single agents. Loewe Volume was used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for additivity is:

I_{Loewe} that satisfies (X/X_{I})+(Y/Y_{I})=1

where X_{I} and Y_{I} are the single agent effective concentrations for the observed combination effect I. For example, if 50% inhibition is achieved separately by 1 mM of drug A or 1 mM of drug B, a combination of 0.5 mM of A and 0.5 mM of B should also inhibit by 50%.

### Synergy Score

To measure combination effects in excess of Loewe additivity, a scalar measure to characterize the strength of synergistic interaction was devised and termed the Synergy Score. The Synergy Score was calculated as:

Synergy Score=log f_{X} log f_{Y}Σ max(0,I_{data})(I_{data}−I_{Loewe})

The fractional inhibition for each component agent and combination point in the matrix was calculated relative to the median of all vehicle-treated control wells. The Synergy Score equation integrates the experimentally-observed activity volume at each point in the matrix in excess of a model surface numerically derived from the activity of the component agents using the Loewe model for additivity. Additional terms in the Synergy Score equation (above) were used to normalize for various dilution factors used for individual agents and to allow for comparison of synergy scores across an entire experiment. The inclusion of positive inhibition gating or an I_{data} multiplier removed noise near the zero effect level, and biases resulted for synergistic interactions at that occur at high activity levels.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This was done by finding the crossing point for each single agent concentration in a dose matrix across the concentrations of the other single agent. Practically, each vertical concentration C_{Y} was held fixed while a bisection algorithm was used to identify the horizontal concentration C_{X} in combination with that vertical dose that gave the chosen effect level in the response surface Z(CX,CY). These concentrations were then connected by linear interpolation to generate the isobologram display. For synergistic interactions, the isobologram contour fall below the additivity threshold and approaches the origin, and an antagonistic interaction would lie above the additivity threshold. The error bars represent the uncertainty arising from the individual data points used to generate the isobologram. The uncertainty for each crossing point was estimated from the response errors using bisection to find the concentrations where Z−σ_{Z}(C_{X},C_{Y}) and Z−σ_{Z}(C_{X},C_{Y}) cross I_{cut}, where σ_{Z} is the standard deviation of the residual error on the effect scale.

Synergy scores for the tested combinations are provided below in Table 2.

In certain embodiments, synergy score of 0 is an additive result. In certain embodiments, synergy score from 0-2 can be considered Additive or just above Additive. In certain embodiments, the higher the synergy score value, the stronger is the synergistic result for the two agents.

TABLE 2

| | DLBCL-GCB | | | | | | | DLBCL-ABC |
|---|---|---|---|---|---|---|---|---|
| | DB | DOHH- 2 | HT | NU-DHL-1 | Pfeiffer | SU-DHL-10 | OCI-Ly19 | OCI-Ly3 |
| Compound I + | 0.39 | 34.70 | 9.94 | 10.40 | 30.10 | 20.70 | 1.32 | 0.25 |
| Ibrutinib | | | | | | | | |

As seen from the data, strong breadth of activity for combinations of compound I with ibrutinib in GCB-DLBCL cell lines were observed. Strong synergy for ibrutinib in DOHH-2 cell line was observed.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims.

### Example 3: Study of a Combination of a PI3K Inhibitor and Ibrutinib in Patients with Chronic Lymphocyctic Leukemia (CLL)

The purpose of this study is to evaluate the safety and effectiveness of Compound I, II, III, or IV (three does: 60 mg, 120 mg, and 150 mg/day) and ibrutinib, in patients with CLL.

Primary Outcome Measures: Determine Acceptable Adverse Events That Are Related to Treatment [Time Frame: 6 months of therapy]. To determine the incidence of adverse events, any potential abnormal laboratory results and any dose-limiting toxicities.

Secondary Outcome Measures: Overall Response Rate [Time Frame: Up to 1 year]. The overall response rate (ORR) in patients with CLL treated with a combination of Compound I, II, III, or IV and ibrutinib.

| Arms | Assigned Interventions |
|---|---|
| Experimental: ibrutinib + Compound I, II, III, or IV | PI3K Inhibitor: Compound I, II, III, or IV |
| Ibrutinib oral daily dose − 420 mg, 280 mg, or 140 mg | A once daily oral agent |
| Compound I, II, III, or IV oral daily dose − 60 mg | Ibrutinib |
| | A once daily oral agent |
| Experimental: ibrutinib + Compound I, II, III, or IV | PI3K Inhibitor: Compound I, II, III, or IV |
| Ibrutinib oral daily dose − 420 mg, 280 mg, or 140 mg | A once daily oral agent |
| Compound I, II, III, or IV oral daily dose − 120 mg | Ibrutinib |
| | A once daily oral agent |
| Experimental: ibrutinib + Compound I, II, III, or IV | PI3K Inhibitor: Compound I, II, III, or IV |
| Ibrutinib oral daily dose − 420 mg, 280 mg, or 140 mg | A once daily oral agent |
| Compound I, II, III, or IV oral daily dose − 150 mg | Ibrutinib |
| | A once daily oral agent |

Patients should not have had exposure to the compounds prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Doses of the compounds may be held or modified for toxicity based on assessments as outlined below. Treatment repeats every 28 days in the absence of unacceptable toxicity. Dose limiting toxicities are determined according to the definitions and standards set by the National Cancer Institute (NCI) Common Terminology for Adverse Events (CTCAE) Version 3.0 (Aug. 9, 2006).

Blood Sampling Serial blood is drawn by direct vein puncture before and after administration of the compound. Venous blood samples (5 mL) for determination of serum concentrations are obtained at about 10 minutes prior to dosing and at approximately the following times after dosing: days 1, 8, and 15. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 8, and 15. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Patient response is also assessed via complete blood cell count and/or marrow biopsy. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). After completion of study treatment, patients are followed periodically for 4 weeks.

### Example 4: Study of a Combination of a PI3K Inhibitor and BGB-3111 in Patients with Relapsed-Refractory Diffuse Large B-Cell Lymphoma (r/r DLBCL)

The purpose of this study is to evaluate the safety and effectiveness of Compound I, II, III, or IV (three does: 30 mg, 45 mg, and 60 mg/day) and BGB-3111, in patients with r/r DLBCL.

Primary Outcome Measures: Determine Acceptable Adverse Events That Are Related to Treatment [Time Frame: 6 months of therapy]. To determine the incidence of adverse events, any potential abnormal laboratory results and any dose-limiting toxicities.

Secondary Outcome Measures: Overall Response Rate [Time Frame: Up to 1 year]. The overall response rate (ORR) in patients with r/r DLBCL treated with a combination of Compound I, II, III, or IV and BGB-3111.

| Arms | Assigned Interventions |
|---|---|
| Experimental: BGB-3111 + Compound I, II, III, or IV | PI3K Inhibitor: Compound I, II, III, or IV |
| BGB-3111 oral daily dose − 160 mg BD or 320 mg QD | A once daily oral agent |
| Compound I, II, III, or IV oral daily dose − 30 mg | BGB-3111 |
| | A daily oral agent |
| Experimental: BGB-3111 + Compound I, II, III, or IV | PI3K Inhibitor: Compound I, II, III, or IV |
| BGB-3111 oral daily dose − 160 mg BD or 320 QD mg | A once daily oral agent |
| Compound I, II, III, or IV oral daily dose − 45 mg | BGB-3111 |
| | A daily oral agent |
| Experimental: BGB-3111 + Compound I, II, III, or IV | PI3K Inhibitor: Compound I, II, III, or IV |
| BGB-3111 oral daily dose − 160 mg BD or 320 mg QD | A once daily oral agent |
| Compound I, II, III, or IV oral daily dose − 60 mg | BGB-3111 |
| | A daily oral agent |

Patients should not have had exposure to the compounds prior to the study entry. Patients must not have received treatment for their cancer within 2 weeks of beginning the trial. Treatments include the use of chemotherapy, hematopoietic growth factors, and biologic therapy such as monoclonal antibodies. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Doses of the compounds may be held or modified for toxicity based on assessments as outlined below. Treatment repeats every 28 days in the absence of unacceptable toxicity. Dose limiting toxicities are determined according to the definitions and standards set by the National Cancer Institute (NCI) Common Terminology for Adverse Events (CTCAE) Version 3.0 (Aug. 9, 2006).

Blood Sampling Serial blood is drawn by direct vein puncture before and after administration of the compound. Venous blood samples (5 mL) for determination of serum concentrations are obtained at about 10 minutes prior to dosing and at approximately the following times after dosing: days 1, 8, and 15. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 8, and 15. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated. Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Patient response is also assessed via complete blood cell count and/or marrow biopsy. Response rates are determined using the RECIST criteria. (Therasse et al, J. Natl. Cancer Inst. 2000 Feb. 2; 92(3):205-16; http://ctep.cancer.gov/forms/TherasseRECISTJNCI.pdf). After completion of study treatment, patients are followed periodically for 4 weeks.

What is claimed is:

1. A method for treating or preventing cancer, comprising administering to a subject in need thereof:
(i) about 30 mg, about 45 mg, or about 60 mg of a compound of Formula (I); 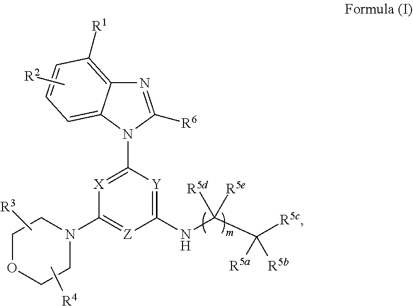
or an enantiomer, a mixture of enantiomers, a mixture of two or more diastereomers, or an isotopic variant thereof; or a pharmaceutically acceptable salt, solvate, hydrate, or prodrug thereof; wherein:
X, Y, and Z are each independently N or CR^{X}, with the proviso that at least two of X, Y, and Z are nitrogen atoms; where R^{X} is hydrogen or C₁₋₆ alkyl;
R¹ and R² are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1d}, —C(O)NR^{1b}R^{1c}, —C(NR^{1a})NR^{1b}R^{1c}, —OR^{1d}, —OC(O)R^{1d}, —OC(O)OR^{1d}, —OC(O)NR^{1b}R^{1c}, —OC(═NR^{1a})NR^{1b}R^{1c}, —OS(O)R^{1a}, —OS(O)₂R^{1d}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c}; wherein each R^{1d}, R^{1b}, R^{1c}, and R^{1d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{1b} and R^{1c} together with the N atom to which they are attached form heterocyclyl;
R³ and R⁴ are each independently hydrogen or C₁₋₆ alkyl; or R³ and R⁴ are linked together to form a bond, C₁₋₆ alkylene, C₁₋₆ heteroalkylene, C₂₋₆ alkenylene, or C₂₋₆ heteroalkenylene;
R^{5a} is (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —S(O)R^{1a}, —S(O)₂R^{1a}, —S(O)NR^{1b}R^{1c}, or —S(O)₂NR^{1b}R^{1c};
R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R⁵ is —(CR^{5f}R^{5g})ₙ—(C₆₋₁₄ aryl) or —(CR^{5f}R^{5g})ₙ-heteroaryl;
R^{5d} and R^{5e} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c};
R^{5f} and R^{5g} are each independently (a) hydrogen or halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}; or —S(O)₂NR^{1b}R^{1c}; or (d) when one occurrence of R^{5f} and one occurrence of R^{5g} are attached to the same carbon atom, the R^{5f} and R^{5g} together with the carbon atom to which they are attached form a C₃₋₁₀ cycloalkyl or heterocyclyl;
R⁶ is hydrogen, C₁₋₆ alkyl, —S—C₁₋₆ alkyl, —S(O)—C₁₋₆ alkyl, or —SO₂—C₁₋₆ alkyl;
m is 0 or 1; and
n is 0, 1, 2, 3, or 4;
wherein each alkyl, alkylene, heteroalkylene, alkenyl, alkenylene, heteroalkenylene, alkynyl, cycloalkyl, aryl, aralkyl, heteroaryl, and heterocyclyl in R¹, R², R³, R⁴, R⁶, R^{X}, R^{1a}, R^{1b}, R^{1c}, R^{1d}, R^{5a}, R^{5b}, R^{5c}, R^{5d}, R^{5e}, R^{5f}, and R^{5g} is optionally substituted with one, two, three, four, or five substituents Q, wherein each substituent Q is independently selected from (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl, each of which is further optionally substituted with one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is further optionally substituted with one, two, three, or four substituents Q^{a}; or (iii) R^{b} and R^{c} together with the N atom to which they are attached form heterocyclyl, which is further optionally substituted with one, two, three, or four substituents Q^{a};
wherein each Q^{a} is independently selected from the group consisting of (a) oxo, cyano, halo, and nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, and heterocyclyl; and (c) —C(O)R^{e}, —C(O)OR^{e}, —C(O)NR^{f}R^{g}, —C(NR^{e})NR^{f}R^{g}, —OR^{e}, —OC(O)R^{e}, —OC(O)OR^{e}, —OC(O)NR^{f}R^{g}, —OC(═NR^{e})NR^{f}R^{g}, —OS(O)R^{e}, —OS(O)₂R^{e}, —OS(O)NR^{f}R^{g}, —OS(O)₂NR^{f}R^{g}, —NR^{f}R^{g}, —NR^{e}C(O)R^{h}, —NR^{e}C(O)OR^{h}, —NR^{e}C(O)NR^{f}R^{g}, —NR^{e}C(═NR^{h})NR^{f}R^{g}, —NR^{e}S(O)R^{h}, —NR^{e}S(O)₂R^{h}, —NR^{e}S(O)NR^{f}R^{g}, —NR^{e}S(O)₂NR^{f}R^{g}, —SR^{e}, —S(O)R^{e}, —S(O)₂R^{e}, —S(O)NR^{f}R^{g}, and —S(O)₂NR^{f}R^{g}; wherein each R^{e}, R^{f}, R^{g}, and R^{h} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (iii) R^{f} and R^{g} together with the N atom to which they are attached form heterocyclyl;
wherein two substituents Q that are adjacent to each other optionally form a C₃₋₁₀ cycloalkenyl, C₆₋₁₄ aryl, heteroaryl, or heterocyclyl, each optionally substituted with one, two, three, or four substituents Q^{a}; and
(ii) about 160 mg or about 320 mg of BGB-3111, or a pharmaceutically acceptable salt thereof,
wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject once per day.

2. The method of claim 1, wherein R^{5b} is (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, or heteroaryl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —S(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

3. The method of claim 1, wherein R^{5a} and R^{5b} are each independently (a) halo; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —SR^{1a}, —S(O)R^{1a}, —S(O)₂R^{1a}, —S(O)NR^{1b}R^{1c}, or —S(O)₂NR^{1b}R^{1c}.

4. The method of claim 3, wherein R^{5a} and R^{5b} are each methyl, optionally substituted with one, two, or three halo(s).

5. The method of any one of claims 1-4, wherein n is 1.

6. The method of any one of claims 1-5, wherein R^{5f} and R^{5g} are each hydrogen.

7. The method of any one of claims 1-4, wherein n is 0.

8. The method of any one of claims 1-7, wherein m is 0.

9. The method of any one of claims 1-8, wherein the compound of Formula (I) is of Formula (XI): 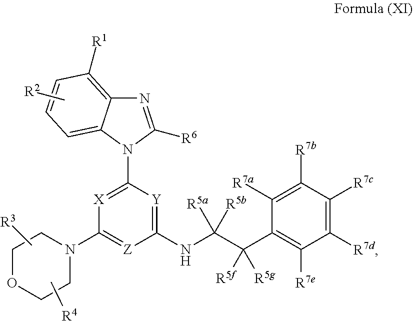
or an enantiomer, a mixture of enantiomers, a mixture of two or more diastereomers, or an isotopic variant thereof; or a pharmaceutically acceptable salt, solvate, hydrate, or prodrug thereof; wherein:
R^{7a}, R^{7b}, R^{7c}, R^{7d}, and R^{7e} are each independently (a) hydrogen, cyano, halo, or nitro; (b) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one, two, three, or four substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, or —S(O)₂NR^{b}R^{c}; or
two of R^{7a}, R^{7b}, R^{7c}, R^{7d}, and R^{7e} that are adjacent to each other form C₃₋₁₀ cycloalkenyl, C₆₋₁₄ aryl, heteroaryl, or heterocyclyl, each optionally substituted with one, two, three, or four substituents Q^{a}.

10. The method of any one of claims 1-9, wherein the compound of Formula (I) is Compound I: 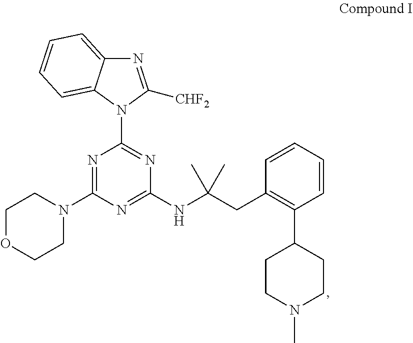 [SMILES: CN1CCC(c2ccccc2CC(C)(C)Nc2nc(N3CCOCC3)nc(-n3c(C(F)F)nc4ccccc43)n2)CC1]
an isotopic variant thereof, a pharmaceutically acceptable salt, solvate, hydrate, or prodrug thereof.

11. The method of any one of claims 1-9, wherein the compound of Formula (I) is Compound II: 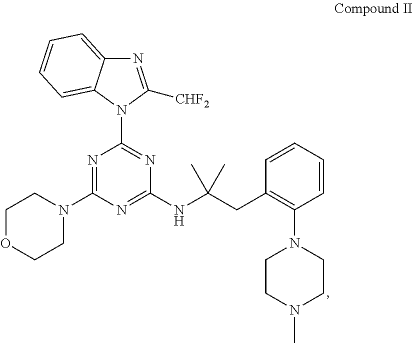 [SMILES: CN1CCN(c2ccccc2CC(C)(C)Nc2nc(N3CCOCC3)nc(-n3c(C(F)F)nc4ccccc43)n2)CC1]
an isotopic variant thereof, a pharmaceutically acceptable salt, solvate, hydrate, or prodrug thereof.

12. The method of any one of claims 1-9, wherein the compound of Formula (I) is Compound III: 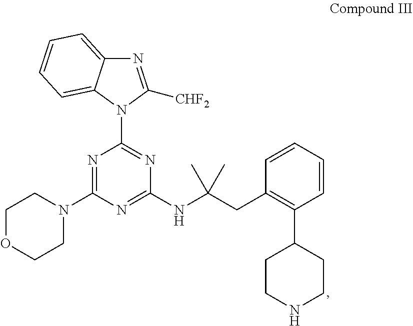 [SMILES: CC(C)(Cc1ccccc1C1CCNCC1)Nc1nc(N2CCOCC2)nc(-n2c(C(F)F)nc3ccccc32)n1]
an isotopic variant thereof, a pharmaceutically acceptable salt, solvate, hydrate, or prodrug thereof.

13. The method of any one of claims 1-9, wherein the compound of Formula (I) is Compound IV: 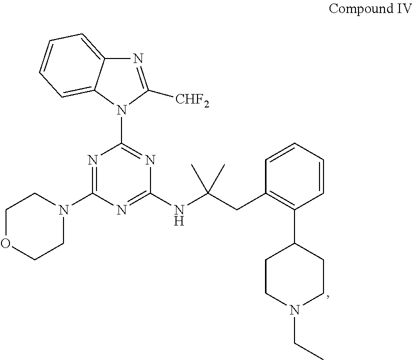 [SMILES: CCN1CCC(c2ccccc2CC(C)(C)Nc2nc(N3CCOCC3)nc(-n3c(C(F)F)nc4ccccc43)n2)CC1]
an isotopic variant thereof, a pharmaceutically acceptable salt, solvate, hydrate, or prodrug thereof.

14. The method of any one of claims 1-9, wherein the compound of Formula (I) is Compound V: 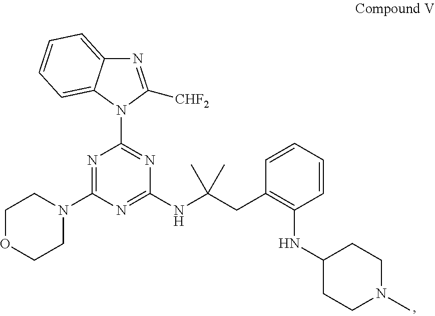 [SMILES: CN1CCC(Nc2ccccc2CC(C)(C)Nc2nc(N3CCOCC3)nc(-n3c(C(F)F)nc4ccccc43)n2)CC1]
an isotopic variant thereof, a pharmaceutically acceptable salt, solvate, hydrate, or prodrug thereof.

15. The method of any one of claims 1-9, wherein the compound of Formula (I) is Compound VI: 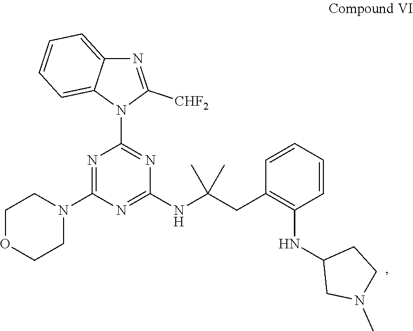 [SMILES: CN1CCC(Nc2ccccc2CC(C)(C)Nc2nc(N3CCOCC3)nc(-n3c(C(F)F)nc4ccccc43)n2)C1]
an isotopic variant thereof, a pharmaceutically acceptable salt, solvate, hydrate, or prodrug thereof.

16. The method of any one of claims 1-9, wherein the compound of Formula (I) is Compound VII: 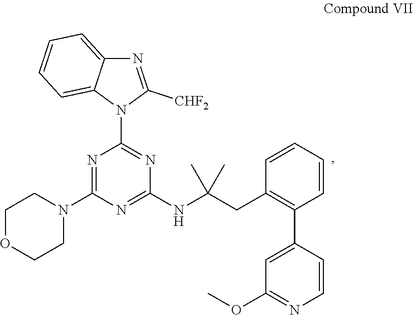 [SMILES: COc1cc(-c2ccccc2CC(C)(C)Nc2nc(N3CCOCC3)nc(-n3c(C(F)F)nc4ccccc43)n2)ccn1]
an isotopic variant thereof, a pharmaceutically acceptable salt, solvate, hydrate, or prodrug thereof.

17. The method of any one of claims 1-9, wherein the compound of Formula (I) is Compound VIII: 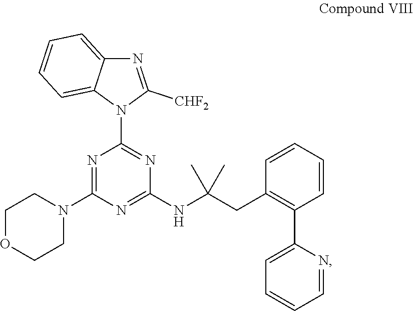 [SMILES: CC(C)(Cc1ccccc1-c1ccccn1)Nc1nc(N2CCOCC2)nc(-n2c(C(F)F)nc3ccccc32)n1]
an isotopic variant thereof, a pharmaceutically acceptable salt, solvate, hydrate, or prodrug thereof.

18. The method of any one of claims 1-9, wherein the compound of Formula (I) is Compound IX: 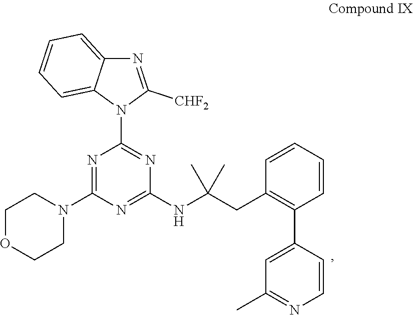 [SMILES: Cc1cc(-c2ccccc2CC(C)(C)Nc2nc(N3CCOCC3)nc(-n3c(C(F)F)nc4ccccc43)n2)ccn1]
an isotopic variant thereof, a pharmaceutically acceptable salt, solvate, hydrate, or prodrug thereof.

19. The method of any one of claims 1-9, wherein the compound of Formula (I) is Compound X: 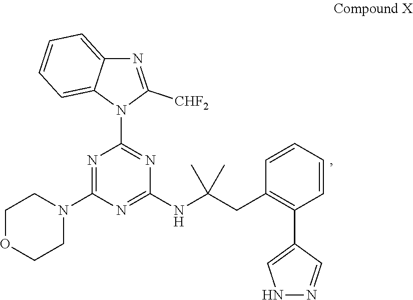 [SMILES: CC(C)(Cc1ccccc1-c1cn[nH]c1)Nc1nc(N2CCOCC2)nc(-n2c(C(F)F)nc3ccccc32)n1]
an isotopic variant thereof, a pharmaceutically acceptable salt, solvate, hydrate, or prodrug thereof.

20. The method of any one of the preceding claims, wherein the cancer is a hematological malignancy.

21. The method of any one of the preceding claims, wherein the cancer is a B-cell malignancy.

22. The method of any one of the preceding claims, wherein the cancer is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

23. The method of any one of the preceding claims, wherein the cancer is chronic lymphocytic leukemia or non-Hodgkin's lymphoma.

24. The method of any one of the preceding claims, wherein the cancer is non-Hodgkin's lymphoma diffuse large B-cell lymphoma (DLBCL).

25. The method of any one of the preceding claims, wherein the cancer is relapsed-refractory diffuse large B-cell lymphoma (r/r DLBCL).

26. The method of claim 24 or 25, wherein the diffuse large B-cell lymphoma is of the activated B-cell (ABC DLBCL) or Germinal center B-cell (GCB DLBCL).

27. The method of any one of the preceding claims, wherein about 30 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

28. The method of any one of claims 1-26, wherein about 45 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

29. The method of any one of claims 1-26, wherein about 60 mg of a compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered to the subject.

30. The method of any one of claims 1-29, wherein about 160 mg of BGB-3111, or a pharmaceutically acceptable salt thereof, is administered to the subject.

31. The method of any one of claims 1-29, wherein about 320 mg of BGB-3111, or a pharmaceutically acceptable salt thereof, is administered to the subject.

32. The method of any one of claims 1-31, wherein BGB-3111, or a pharmaceutically acceptable salt thereof, is administered to the subject once per day or twice per day.

33. The method of any one of claims 1-31, wherein BGB-3111, or a pharmaceutically acceptable salt thereof, is administered to the subject once per day

34. The method of any one of claims 1-31, wherein BGB-3111, or a pharmaceutically acceptable salt thereof, is administered to the subject twice per day.

35. The method of any one of claims 1-31, wherein about 160 mg of BGB-3111, or a pharmaceutically acceptable salt thereof, is administered to the subject twice per day.

36. The method of any one of claims 1-31, wherein about 320 mg of BGB-3111, or a pharmaceutically acceptable salt thereof, is administered to the subject once per day.

37. The method of any one of claims 1-36, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, and BGB-3111, or a pharmaceutically acceptable salt thereof, are administered simultaneously, approximately simultaneously, or sequentially in any order.

38. The method of any one of claims 1-36, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, and BGB-3111, or a pharmaceutically acceptable salt thereof, are administered simultaneously or approximately simultaneously.

39. The method of any one of claims 1-36, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, and BGB-3111, or a pharmaceutically acceptable salt thereof, are administered sequentially.

40. The method of claim 39, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered before BGB-3111, or a pharmaceutically acceptable salt thereof.

41. The method of claim 39, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is administered after BGB-3111, or a pharmaceutically acceptable salt thereof.

42. The method of any one of the preceding claims, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is formulated as a tablet or capsule.

43. The method of any one of the preceding claims, wherein BGB-3111, or a pharmaceutically acceptable salt thereof, is formulated as a tablet or capsule.

44. The method of any one of claims 1-38, wherein the compound of Formula (I), or an enantiomer, a mixture of enantiomers, a mixture of two or more diastereomers, or an isotopic variant thereof; or a pharmaceutically acceptable salt, solvate, hydrate, or prodrug thereof, is co-formulated with BGB-3111, or a pharmaceutically acceptable salt thereof.

45. A pharmaceutical composition, comprising Compound I: 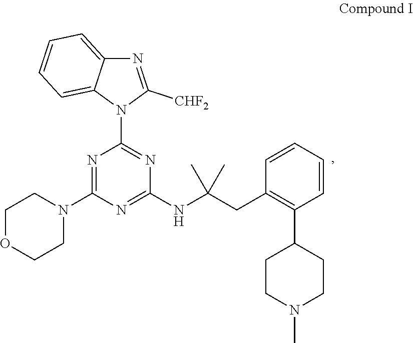 [SMILES: CN1CCC(c2ccccc2CC(C)(C)Nc2nc(N3CCOCC3)nc(-n3c(C(F)F)nc4ccccc43)n2)CC1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

46. A pharmaceutical composition, comprising Compound II: 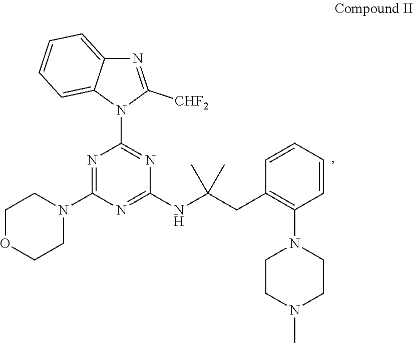 [SMILES: CN1CCN(c2ccccc2CC(C)(C)Nc2nc(N3CCOCC3)nc(-n3c(C(F)F)nc4ccccc43)n2)CC1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

47. A pharmaceutical composition, comprising Compound III: 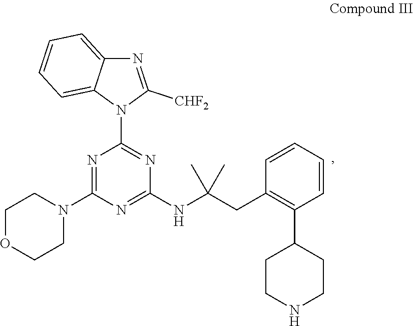 [SMILES: CC(C)(Cc1ccccc1C1CCNCC1)Nc1nc(N2CCOCC2)nc(-n2c(C(F)F)nc3ccccc32)n1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

48. A pharmaceutical composition, comprising Compound IV: 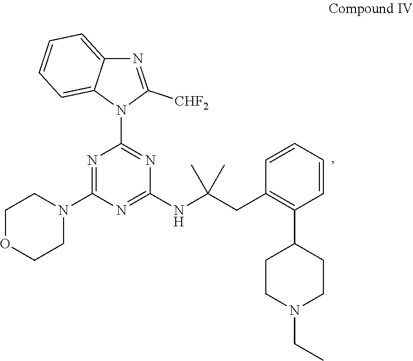 [SMILES: CCN1CCC(c2ccccc2CC(C)(C)Nc2nc(N3CCOCC3)nc(-n3c(C(F)F)nc4ccccc43)n2)CC1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

49. A pharmaceutical composition, comprising Compound V: 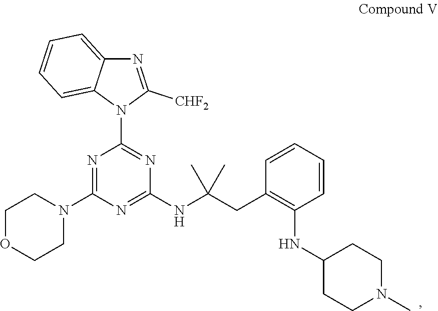 [SMILES: CN1CCC(Nc2ccccc2CC(C)(C)Nc2nc(N3CCOCC3)nc(-n3c(C(F)F)nc4ccccc43)n2)CC1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

50. A pharmaceutical composition, comprising Compound VI: 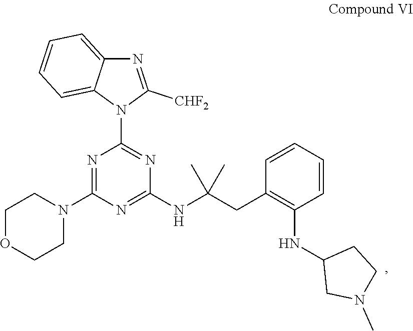 [SMILES: CN1CCC(Nc2ccccc2CC(C)(C)Nc2nc(N3CCOCC3)nc(-n3c(C(F)F)nc4ccccc43)n2)C1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

51. A pharmaceutical composition, comprising Compound VII: 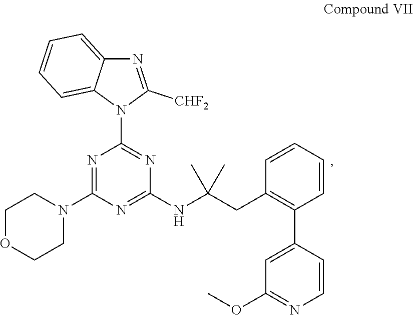 [SMILES: COc1cc(-c2ccccc2CC(C)(C)Nc2nc(N3CCOCC3)nc(-n3c(C(F)F)nc4ccccc43)n2)ccn1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

52. A pharmaceutical composition, comprising Compound VIII: 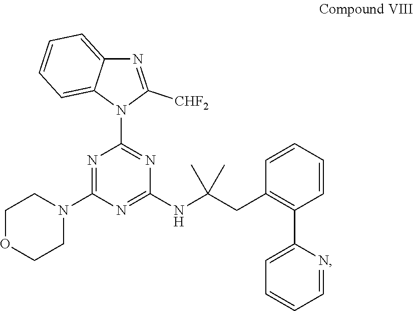 [SMILES: CC(C)(Cc1ccccc1-c1ccccn1)Nc1nc(N2CCOCC2)nc(-n2c(C(F)F)nc3ccccc32)n1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

53. A pharmaceutical composition, comprising Compound IX: 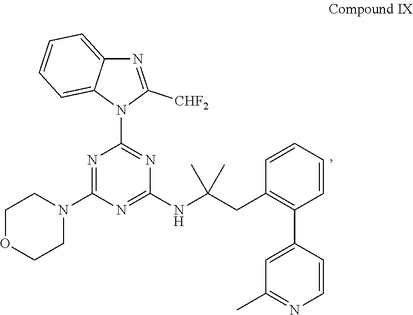 [SMILES: Cc1cc(-c2ccccc2CC(C)(C)Nc2nc(N3CCOCC3)nc(-n3c(C(F)F)nc4ccccc43)n2)ccn1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

54. A pharmaceutical composition, comprising Compound X: 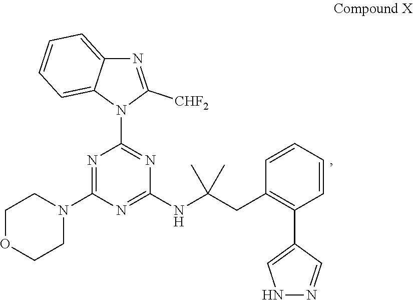 [SMILES: CC(C)(Cc1ccccc1-c1cn[nH]c1)Nc1nc(N2CCOCC2)nc(-n2c(C(F)F)nc3ccccc32)n1]
or an isotopic variant thereof; or a pharmaceutically acceptable salt, solvate, hydrate, or prodrug thereof; BGB-3111, or a pharmaceutically acceptable salt thereof; and a pharmaceutically acceptable excipient.

55. A method for treating or preventing cancer, comprising administering to a subject in need thereof an effective amount of the pharmaceutical composition of any one of claims 45-54.

56. The method of claim 55, wherein the cancer is a hematological malignancy.

57. The method of claim 55, wherein the cancer is a B-cell malignancy.

58. The method of claim 55, wherein the cancer is acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

59. The method of claim 55, wherein the cancer is chronic lymphocytic leukemia or non-Hodgkin's lymphoma.

60. The method of claim 55, wherein the cancer is non-Hodgkin's lymphoma diffuse large B-cell lymphoma (DLBCL).

61. The method of claim 55, wherein the cancer is relapsed-refractory diffuse large B-cell lymphoma (r/r DLBCL).

62. The method of claim 60 or 61, wherein the diffuse large B-cell lymphoma is of the activated B-cell (ABC DLBCL) or Germinal center B-cell (GCB DLBCL).
